(19) United States
(12) Patent Application Publication
(10) Pub. No.: US 2024/0201196 A1
(43) Pub. Date: Jun. 20, 2024

(54) NON-COVALENT HALOTAG LIGANDS

(71) Applicant: MAX-PLANCK-GESELLSCHAFT ZUR FÖRDERUNG DER WISSENSCHAFTEN E. V., München (DE)

(72) Inventors: Kai JOHNSSON, Heidelberg (DE); Julien HIBLOT, Heidelberg (DE); Julian KOMPA, Rödermark (DE)

(73) Assignee: MAX-PLANCK-GESELLSCHAFT ZUR FÖRDERUNG DER WISSENSCHAFTEN E. V., München (DE)

(21) Appl. No.: 18/554,819

(22) PCT Filed: May 2, 2022
(86) PCT No.: PCT/EP2022/061679

§ 371 Date: Oct. 11, 2023

(30) Foreign Application Priority Data
Apr. 30, 2021 (EP) 21171702.0
Oct. 27, 2021 (EP) 21205120.5

(51) Int. Cl. G01N 33/58; C07K 14/00

(52) U.S. Cl. CPC G01N 33/581; C07K 14/001; G01N 2333/90241

(57) ABSTRACT

A first aspect of the invention relates to a non-covalently-HaloTag-binding compound characterized by the general formula D-L-T (I), wherein D is or comprises a functional moiety Z, particularly a fluorescent dye, or D is a linkable moiety (i.e. a moiety that can be coupled to other functional groups), L is a linear linker of 10-15 atoms in length, and T is a moiety selected from the group comprising methylamine, methylsulfonamide, acetamide, or their respective fluorinated analogues, azide, or hydroxyl. Another aspect of the invention relates to a HaloTag variant wherein position D106 of the HaloTag7 sequence is exchanged for a proteinogenic amino acid different from D. The variant has a different binding specificity for HaloTag substrates compared to the non-variant Halotag polypeptide. Yet another aspect of the invention relates to kits comprising polypeptides or nucleic acids and the non-covalently-HaloTag-binding compound according to the invention.

### FIELD OF THE INVENTION

The present invention relates to small molecule compounds that non-covalently bind to the common HaloTag haloalkane dehalogenase derivative, and to variants of the halotag peptide having binding selectivity for particular compounds.

This application claims the benefit of priority of European patent applications EP21171702.0 filed 30 Apr. 2021 and EP21205120.5 filed 17. Oct. 2021 2021, both of which are incorporated by reference herein.

### BACKGROUND

Fluorescence microscopy is a key tool to directly visualize the function or architecture of biological structures. The majority of fluorescence microscopy techniques investigate the subcellular localization of proteins. Consequently, progress in super-resolution microscopy is tightly bound to the development of methods to fluorescently mark proteins of interest. Genetically encoded protein tags, due to their non-invasive nature, open the door for live-cell microscopy. Recent labelling approaches rely on engineered self-labelling protein (SLP) tags. A mutant of the haloalkane dehalogenase DhaA (HaloTag) is one of the most established representatives of such SLPs. HaloTag is a self-labelling protein tag derived from a bacterial enzyme, designed to covalently bind to a synthetic ligand. It generates a covalent link to a small, synthetic bio-orthogonal substrate by reacting with chloroalkane substrates that are inert under conditions. substrates are based cellular HaloTag on 2-[2-[(6-chlorohexyl)oxy]ethoxy]ethanamine linker (HaloTag-Ligand, HTL). The covalent link between the HaloTag protein and its ligand leads to photobleaching of the bound fluorophore. The combination of tag-based staining with non-covalent fluorescent ligands, leading to replacement of photobleached probes from the reservoir of unbound dyes, would facilitate prolonged image acquisitions. The objective of the present invention is to provide non-covalent ligands of the HaloTag protein, novel HaloTag protein variants useful for non-covalent attachment, and methods for their use This objective is attained by the subject matter defined in the independent claims of this specification, with advantageous embodiments set forth in the specification, examples and dependent claims.

### SUMMARY OF THE INVENTION

A first aspect of the invention relates to a non-covalently-HaloTag-binding compound characterized by the general formula D-L-T (I), wherein
D is or comprises a functional moiety Z, or
D is a linkable moiety (i.e. a moiety that can be coupled to other functional groups)
   L is a linear linker of 10-15 atoms in length, and
   T is a moiety selected from the group comprising methylamine, methylsulfonamide, acetamide, or their respective fluorinated analogues, azide, or hydroxyl.

Another aspect of the invention relates to a HaloTag variant wherein position D106 of the HaloTag7 sequence or its homologues is exchanged for a proteinogenic amino acid different from D. The variant has a different binding specificity for the compound as defined according to the first aspect compared to the non-variant Halotag polypeptide.

Yet another aspect of the invention relates to kits comprising polypeptides or nucleic acids and the non-covalently-HaloTag-binding compound according to the invention.

### TERMS AND DEFINITIONS

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising”, “having”, “containing”, and “including”, and other similar forms, and grammatical equivalents thereof, as used herein, are intended to be equivalent in meaning and to be open-ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a”, “or” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term Halo Tag polypeptide in the context of the present specification relates to a modified haloalkane dehalogenase as commercialized by the Promega corporation under the HaloTag trademark. The original HaloTag is a 297-residue polypeptide derived from a bacterial haloalkane dehalogenase enzyme, designed to covalently bind to a synthetic ligand. The HaloTag can be fused to various proteins of interest. The synthetic ligand can be selected from a number of commercially available ligands. The system is designed to facilitate visualization of the subcellular localization of a protein of interest, immobilization of a protein of interest, or capture of the binding partners of a protein of interest within its biochemical environment.

The term catalytically functional Halo Tag polypeptide in the context of the present specification relates to a HaloTag polypeptide having haloalkane dehalogenase enzymatic activity.

The term wildtype Halo Tag polypeptide in the context of the present specification relates to the polypeptide sequence capable of covalently linking to a haloalkane moiety, which has an aspartate (D) in a position homologous to position 106 of halotag7. The term wildtype is used in distinction to a variant that has no D in position 106, and which has a different binding specificity as laid out further herein.

### Sequences

Sequences similar or homologous (e.g., at least about 70% sequence identity) to the sequences disclosed herein, and which have the same functional characteristics as laid out further herein below, are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher.

In the context of the present specification, the terms sequence identity and percentage of sequence identity refer to a single quantitative parameter representing the result of a sequence comparison determined by comparing two aligned sequences position by position. Methods for alignment of sequences for comparison are well-known in the art. Alignment of sequences for comparison may be conducted by the local homology algorithm of Smith and Waterman, Adv. Appl. Math. 2: 482 (1981), by the global alignment algorithm of Needleman and Wunsch, J. Mol. Biol. 48: 443 (1970), by the search for similarity method of Pearson and Lipman, Proc. Nat. Acad. Sci. 85: 2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1 .- 2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively.

Reference to identical sequences without specification of a percentage value implies 100% identical sequences (i.e. the same sequence).

### General Biochemistry: Peptides, Amino Acid Sequences

The term polypeptide in the context of the present specification relates to a molecule consisting of 50 or more amino acids that form a linear chain wherein the amino acids are connected by peptide bonds. The amino acid sequence of a polypeptide may represent the amino acid sequence of a whole (as found physiologically) protein or fragments thereof. The term “polypeptides” and “protein” are used interchangeably herein and include proteins and fragments thereof. Polypeptides are disclosed herein as amino acid residue sequences.

The term peptide in the context of the present specification relates to a molecule consisting of up to 50 amino acids, in particular 8 to 30 amino acids, more particularly 8 to 15amino acids, that form a linear chain wherein the amino acids are connected by peptide bonds.

Amino acid residue sequences are given from amino to carboxyl terminus. Capital letters for sequence positions refer to L-amino acids in the one-letter code (Stryer, Biochemistry, 3^{rd} ed. p. 21). Lower case letters for amino acid sequence positions refer to the corresponding D- or (2R)-amino acids. Sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (lle, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

The term variant refers to a polypeptide that differs from a reference polypeptide, but retains essential properties. A typical variant of a polypeptide differs in its primary amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

The term fluorescent dye or fluorescent organic dye moiety in the context of the present specification relates to a small molecule capable of fluorescence in the visible or near infrared spectrum.

### Organic Chemistry

The term C₁-C₄ alkyl in the context of the present specification relates to a saturated linear or branched hydrocarbon having 1, 2, 3 or 4 carbon atoms, wherein in certain embodiments one carbon-carbon bond may be unsaturated and one CH₂ moiety may be exchanged for oxygen (ether bridge) or nitrogen (NH, or NR with R being methyl, ethyl, or propyl; amino bridge). Non-limiting examples for a C₁-C₄ alkyl are methyl, ethyl, propyl, prop-2-enyl, n-butyl, 2-methylpropyl, tert-butyl, but-3-enyl, prop-2-inyl and but-3-inyl. In certain embodiments, a C₁-C₄ alkyl is a methyl, ethyl, propyl or butyl moiety.

A C₁-C₆ alkyl in the context of the present specification relates to a saturated linear or branched hydrocarbon having 1, 2, 3, 4, 5 or 6 carbon atoms, wherein one carbon-carbon bond may be unsaturated and/or one CH₂ moiety may be exchanged for oxygen (ether bridge) or nitrogen (NH, or NR with R being methyl, ethyl, or propyl; amino bridge). Non-limiting examples for a C₁-C₆ alkyl include the examples given for C₁-C₄ alkyl above, and additionally 3-methylbut-2-enyl, 2-methylbut-3-enyl, 3-methylbut-3-enyl, n-pentyl, 2-methylbutyl, 3-methylbutyl, 1,1-dimethylpropyl, 1,2-dimethylpropyl, 1,2-dimethylpropyl, pent-4-inyl, 3-methyl-2-pentyl, and 4-methyl-2-pentyl. In certain embodiments, a C₅ alkyl is a pentyl or cyclopentyl moiety and a C₆ alkyl is a hexyl or cyclohexyl moiety.

The term C₄-C₇ cycloalkyl in the context of the present specification relates to a saturated hydrocarbon ring having 4, 5, 6 or 7 carbon atoms, wherein in certain embodiments, one carbon-carbon bond may be unsaturated and/or one CH₂ moiety may be exchanged for oxygen (ether bridge) or nitrogen (NH, or NR with R being methyl, ethyl, or propyl; amino bridge). Non-limiting examples of a C₄-C₇ cycloalkyl moiety include cyclobutyl (—C₄H₇), cyclopentenyl (C₅H₉), and cyclohexenyl (C₆H₁₁) moieties. In certain embodiments, a cycloalkyl is substituted by one C₁ to C₄ unsubstituted alkyl moiety. In certain embodiments, a cycloalkyl is substituted by more than one C₁ to C₄ unsubstituted alkyl moieties.

The term unsubstituted Cₙ alkyl when used herein in the narrowest sense relates to the moiety —CₙH₂ₙ- if used as a bridge between moieties of the molecule, or —CₙH₂ₙ₊₁ if used in the context of a terminal moiety. It may still contain fewer H atoms if a cyclical structure or one or more (non-aromatic) double bonds are present.

The term Cₙ alkylene in the context of the present specification relates to a saturated linear or branched hydrocarbon comprising one or more double bonds. An unsubstituted alkylene consists of C and H only. A substituted alkylene may comprise substituents as defined herein for substituted alkyl.

The term Cₙ alkylyne in the context of the present specification relates to a saturated linear or branched hydrocarbon comprising one or more triple bonds and may also comprise one or more double bonds in addition to the triple bond(s). An unsubstituted alkylyne consists of C and H only. A substituted alkylyne may comprise substituents as defined herein for substituted alkyl.

The terms unsubstituted Cₙ alkyl and substituted Cₙ alkyl include a linear alkyl comprising or being linked to a cyclical structure, for example a cyclopropane, cyclobutane, cyclopentane or cyclohexane moiety, unsubstituted or substituted depending on the annotation or the context of mention, having linear alkyl substitutions. The total number of carbon and—where appropriate—N, O or other hetero atom in the linear chain or cyclical structure adds up to n.

Where used in the context of chemical formulae, the following abbreviations may be used: Me is methyl CH₃, Et is ethyl —CH₂CH₃, Prop is propyl —(CH₂)₂CH₃ (n-propyl, n-pr) or —CH(CH₃)₂ (iso-propyl, i-pr), but is butyl —C₄H₉, —(CH₂)₃CH₃, —CHCH₃CH₂CH₃, —CH₂CH(CH₃)₂ or —C(CH₃)₃.

The term substituted alkyl in its broadest sense refers to an alkyl as defined above in the broadest sense, which is covalently linked to an atom that is not carbon or hydrogen, particularly to an atom selected from N, O, F, B, Si, P, S, Cl, Br and I, which itself may be—if applicable—linked to one or several other atoms of this group, or to hydrogen, or to an unsaturated or saturated hydrocarbon (alkyl or aryl in their broadest sense). In a narrower sense, substituted alkyl refers to an alkyl as defined above in the broadest sense that is substituted in one or several carbon atoms by groups selected from amine NH₂, alkylamine NHR, imide NH, alkylimide NR, amino(carboxyalkyl) NHCOR or NRCOR, hydroxyl OH, oxyalkyl OR, oxy(carboxyalkyl) OCOR, carbonyl O and its ketal or acetal (OR)₂, nitril CN, isonitril NC, cyanate CNO, isocyanate NCO, thiocyanate CNS, isothiocyanate NCS, fluoride F, choride Cl, bromide Br, iodide I, phosphonate PO₃H₂, PO₃R₂, phosphate OPO₃H₂ and OPO₃R₂, sulfhydryl SH, suflalkyl SR, sulfoxide SOR, sulfonyl SO₂R, sulfanylamide SO₂NHR, sulfate SO₃H and sulfate ester SO₃R, wherein the R substituent as used in the current paragraph, different from other uses assigned to R in the body of the specification, is itself an unsubstituted or substituted C₁ to C₁₂ alkyl in its broadest sense, and in a narrower sense, R is methyl, ethyl or propyl unless otherwise specified.

The term amino substituted alkyl or hydroxyl substituted alkyl refers to an alkyl according to the above definition that is modified by one or several amine or hydroxyl groups NH₂, NHR, NR₂ or OH, wherein the R substituent as used in the current paragraph, different from other uses assigned to R in the body of the specification, is itself an unsubstituted or substituted C₁ to C₁₂ alkyl in its broadest sense, and in a narrower sense, R is methyl, ethyl or propyl unless otherwise specified. An alkyl having more than one carbon may comprise more than one amine or hydroxyl. Unless otherwise specified, the term “substituted alkyl” refers to alkyl in which each C is only substituted by at most one amine or hydroxyl group, in addition to bonds to the alkyl chain, terminal methyl, or hydrogen.

The term carboxyl substituted alkyl refers to an alkyl according to the above definition that is modified by one or several carboxyl groups COOH, or derivatives thereof, particularly carboxylamides CONH₂, CONHR and CONR₂, or carboxylic esters COOR, with R having the meaning as laid out in the preceding paragraph and different from other meanings assigned to R in the body of this specification.

Non-limiting examples of amino-substituted alkyl include —CH₂NH₂, —CH₂NHMe, —CH₂NHEt, —CH₂CH₂NH₂, —CH₂CH₂NHMe, —CH₂CH₂NHEt, —(CH₂)₃NH₂, —(CH₂)₃NHMe, —(CH₂)₃NHEt, —CH₂CH(NH₂)CH₃, —CH₂CH(NHMe)CH₃, —CH₂CH(NHEt)CH₃, —(CH₂)₃CH₂NH₂, —CH₂)₃CH₂NHMe, —(CH₂)₃CH₂NHEt, —CH(CH₂NH₂)CH₂CH₃, —CH(CH₂NHMe)CH₂CH₃, —CH(CH₂NHEt)CH₂CH₃, —CH₂CH(CH₂NH₂)CH₃, —CH₂CH(CH₂NHMe)CH₃, —CH₂CH(CH₂NHEt)CH₃, —CH(NH₂)(CH₂)₂NH₂, —CH(NHMe)(CH₂)₂NHMe, —CH(NHEt)(CH₂)₂NHEt, —CH₂CH(NH₂)CH₂NH₂, —CH₂CH(NHMe)CH₂NHMe, —CH₂CH(NHEt)CH₂NHEt, —CH₂CH(NH₂)(CH₂)₂NH₂, —CH₂CH(NHMe)(CH₂)₂NHMe, —CH₂CH(NHEt)(CH₂)₂NHEt, —CH₂CH(CH₂NH₂)₂, —CH₂CH(CH₂NHMe)₂ and —CH₂CH(CH₂NHEt)₂ for terminal moieties and —CH₂CHNH₂—, —CH₂CHNHMe—, —CH₂CHNHEt- for an amino substituted alkyl moiety bridging two other moieties.

Non-limiting examples of hydroxy-substituted alkyl include —CH₂OH, —(CH₂)₂OH, —(CH₂)₃OH, —CH₂CH(OH)CH₃, —(CH₂)₄OH, —CH(CH₂OH)CH₂CH₃, —CH₂CH(CH₂OH)CH₃, —CH(OH)(CH₂)₂OH, —CH₂CH(OH)CH₂OH, —CH₂CH(OH)(CH₂)₂OH and —CH₂CH(CH₂OH)₂ for terminal moieties and —CHOH—, —CH₂CHOH—, —CH₂CH(OH)CH₂—, —(CH₂)₂CHOHCH₂—, —CH(CH₂OH)CH₂CH₂—, —CH₂CH(CH₂OH)CH₂—, —CH(OH)(CH₂CHOH—, —CH₂CH(OH)CH₂OH, —CH₂CH(OH)(CH₂)₂OH and —CH₂CHCH₂OHCHOH— for a hydroxyl substituted alkyl moiety bridging two other moieties.

The term halogen-substituted alkyl refers to an alkyl according to the above definition that is modified by one or several halogen atoms selected (independently) from F, Cl, Br, I. The term aryl in the context of the present specification relates to a cyclic aromatic C₅-C₁₀ hydrocarbon that may comprise a heteroatom (e.g. N, O, S). Examples of aryl include, without being restricted to, phenyl and naphthyl, and any heteroaryl. A heteroaryl is an aryl that comprises one or several nitrogen, oxygen and/or sulphur atoms. Examples for heteroaryl include, without being restricted to, pyrrole, thiophene, furan, imidazole, pyrazole, thiazole, oxazole, pyridine, pyrimidine, thiazin, quinoline, benzofuran and indole. An aryl or a heteroaryl in the context of the specification additionally may be substituted by one or more alkyl groups.

A carboxylic ester is a group —CO₂R, with R being defined further in the description. A carboxylic amide is a group —CONHR, with R being defined further in the description.

### DETAILED DESCRIPTION OF THE INVENTION

A first aspect of the invention relates to a non-covalently-HaloTag-binding compound characterized by the general formula D-L-T (I).

D is or comprises a functional moiety Z, or D is a linkable moiety (i.e. a moiety that can be coupled to other functional groups).

L is a linear linker of 10-15 atoms in length. L can be any molecular link between D and T, as long as it facilitates entry of T into the active site of the HaloTag polypeptide, and allows non-covalent interaction of T with the HaloTag.

In certain embodiments, L comprises alkyl, trans-alkylene, and/or ether moieties and optionally one or several methyl substituents. In certain embodiments, L is 10-11 atoms in length. In certain embodiments, L is an unbranched linker that does not comprise methyl substituents. In certain particular embodiments, L essentially consists of alkyl, trans-alkylene, and/or ether moieties and optionally one or several methyl substituents.

According to the first aspect, T is a moiety selected from the group comprising methylamine, monofluormethylamine, difluormethylamine, trifluormethylamine, methylsulfonamide, monofluormethylsulfonamide, difluormethylsulfonamide, trifluormethylsulfonamide, azide, acetamide, monofluoracetamide, difluoracetamide, and trifluoracetamide.

In certain particular embodiments of this first aspect of the invention, T is a moiety selected from the group comprising methylamine, trifluormethylsulfonamide and methylsulfonamide.

The functional moiety Z can be selected from:
an organic dye moiety characterized by a molecular mass of between 300 g/mol and 1300 g/mol, particularly a fluorescent organic dye moiety;
an affinity purification ligand,
a pharmaceutical drug or a pharmaceutical drug candidate,
an oligopeptide or a polypeptide,
a nanoparticle,
a solid surface or a matrix polymer,
a nucleic acid oligomer,
a carbohydrate,
a lipid,
a spectroscopic probe,
a sensor, particularly a fluorescent sensor,
a natural product, particularly a vitamin, provitamin, enzymatic co-factor having a molecular mass of <1000 g/mol, and
a synthetic ligand binding to a biomolecule with an affinity of at least 100 μM.

In certain embodiments, the functional moiety Z is selected from:
an organic dye moiety characterized by a molecular mass of between 300 g/mol and 1300 g/mol, particularly a fluorescent organic dye moiety;
an affinity purification ligand,
a pharmaceutical drug or a pharmaceutical drug candidate, and
a solid surface or a matrix polymer.

### Z can be a Dye Molecule

In certain particular embodiments, the functional moiety Z is a spectroscopic probe (dye molecule). In certain embodiments, the functional moiety Z is a probe that can be excited by electromagnetic radiation and where excitation can subsequently be used for its detection in biological samples.

In certain embodiments, the functional moiety Z is an organic dye moiety characterized by a molecular mass of between 300 g/mol and 1300 g/mol. In certain embodiments, the functional moiety Z is a fluorescent organic dye moiety. The HaloTag system has been explored deeply for applications in which a fluorescent dye molecule is coupled, and the present invention significantly extends these applications, for example in the realm of ultrahigh-resolution fluorescence microscopy (PAINT, STED).

### Z Can Be Used to Separate Molecules on a Solid Phase, By Coupling to the Analyte or Solid Phase

Other applications for non-covalent, highly specific and reversible interaction of the compounds disclosed herein include purification.

In certain embodiments, the functional moiety Z is an affinity purification ligand. In certain embodiments, the affinity purification ligand is selected from a protein- or peptide-tag, and a small-molecule-tag. In certain embodiments, the affinity purification ligand is selected from biotin, streptavidin, calmodulin, FLAG-tag, HA-tag, His-tag, myc-tag, NE-tag, strep-tag, T7-tag,

S-tag, Fc-tag, MBP-tag, CBP-tag, PDZ-tag, GST-tag, CLIP-tag, and SNAP-tag.

In certain embodiments, the functional moiety Z is a solid surface or a matrix polymer. Particular examples include magnetic particles, superparamagnetic particles, or matrix materials for chromatographic columns on which proteins bearing a HaloTag may be separated.

### Z Can be a Drug or Drug Candidate Molecule

In certain embodiments, the functional moiety Z is a pharmaceutical drug or a pharmaceutical drug candidate fulfilling the so-called Lipinski rule-of-five. The Lipinski rule states that the pharmaceutical drug or a pharmaceutical drug candidate has no more than 5 hydrogen bond donors, no more than 10 hydrogen bond acceptors, a molecular mass of less than 500 daltons, and an octanol-water partition coefficient (log P) that does not exceed 5.

In certain embodiments, the functional moiety Z is an oligopeptide or a polypeptide.

In certain embodiments, the functional moiety Z is a nanoparticle. A nanoparticle is a particle of matter that is between 1 and 100 nm in diameter. Advantageous applications of nanoparticle technology include those using gold nanoparticles and quantum dots, nanoscale semiconductor materials. Many methods for covalent linking of organic linkers to nanoparticles are known in the art, which include but are not limited to sulfur (SH) mediated bonds to metal surfaces.

In certain embodiments, the functional moiety Z is a nucleic acid oligomer. The tagged nucleic acid could thus be directed at, and reversibly attached to, a protein or another structure bearing the HaloTag.

In certain embodiments, the functional moiety Z is a carbohydrate.

In certain embodiments, the functional moiety Z is a lipid.

In certain embodiments, the functional moiety Z is a sensor. In certain embodiments, the functional moiety Z is a fluorescent sensor. In certain embodiments, the fluorescent sensor is an environment-sensitive or analyte-binding small chemical to detect the presence of a particular substance of interest by the use of fluorescence. In certain embodiments, the fluorescent sensor works via a fluorescent readout change to indicate changes of an analyte concentration or other external factor of interest.

In certain embodiments, the functional moiety Z is a natural product. In certain embodiments, the functional moiety Z is a metabolite, a vitamin, a provitamin, or an enzymatic co-factor having a molecular mass of <1000 g/mol. In certain embodiments, the functional moiety Z is a vitamin, a provitamin, or an enzymatic co-factor having a molecular mass of <1000 g/mol.

### Z can be a Reactive Link to a Biomolecule

In certain embodiments, the functional moiety Z is a synthetic ligand binding to a biomolecule with an affinity of at least 100 μM. Such ligand allows coupling of a biomolecule. One application is the commercial provision of pre-synthesized non-covalent HaloTag ligands, to which biomolecules or reactive partners can be coupled by selective, highly reactive reaction partners (i.e. “click chemistry” partners).

The linkable moiety of D is selected from
an unprotected or protected amine moiety,
a sulfonamide moiety,
an N₃ moiety,
an alkyne moiety,
a carboxylic acid or an activated form of a carboxylic acid, particularly an N-hydroxysuccinimide moiety,
an ester moiety,
an aldehyde,
a thiol, and
an isothiocyanate.

In certain embodiments, the linkable moiety of D is an unprotected or protected amine moiety, facilitating the attachment of the compound in a ready-to-use format to a suitable chemical function on a substrate, for example by attaching it to an activated carboxylic acid on the substrate. In turn, certain embodiments provide the linkable moiety of D as a carboxylic acid or an activated form of a carboxylic acid, rendering easy attachment to an amine or hydroxyl moiety on a substrate. In certain embodiments, the linkable moiety of D is an N-hydroxysuccinimide moiety.

In certain embodiments, the linkable moiety of D is a sulfonamide moiety. Certain sulfonamides are amenable to Huisgen 1,3-dipolar cycloaddition.

In certain embodiments, the linkable moiety of D is an N₃ moiety. In certain embodiments, the linkable moiety of D is an alkyne moiety. Azide groups react with carbon-carbon triple bonds by way of 1,3-dipolar cycloaddition. The catalyzed coupling of an alkyne to an azide facilitates the attachment of the compound to a broad range of substrates by simple “click chemistry” reaction.

In certain embodiments, the linkable moiety of D is an ester moiety or an activated ester, readily reacting with nucleophiles such as amines. In certain embodiments, the linkable moiety of D is an aldehyde, which can, inter alia, form Schiff base adducts with amines. In certain embodiments, the linkable moiety of D is a thiol. In certain embodiments, the linkable moiety of D is an isothiocyanate. Isothiocyanates can undergo click-type reactions with thiols.

### The Moiety T Replacing the Chlorine of “Classical” Halo Tag Substrates

The inventors have found that methylsulfonamide and methylamine can replace the chlorine of the classical HaloTag substrate to lead to tightly binding, yet non-covaltently interacting ligands of the commercial HaloTag protein tag. Hydroxyl as a replacement of chlorine also binds, but binds better to a variant of HaloTag wherein position 106 is altered.

The inventors have furthermore established that instead of methylamine, a moiety selected from monofluormethylamine, difluormethylamine and trifluormethylamine may be used. Sulfonamide may be replaced by a moiety selected from monofluormethylsulfonamide, difluormethylsulfonamide and trifluormethylsulfonamide. Substrates in which a moiety selected from azide, acetamide, monofluoracetamide, difluoracetamide, and trifluoracetamide replaces the chlorine also bind to the classical HaloTag in a non-covalent manner.

An alternative of the first aspect relates to a non-covalently-HaloTag-binding compound characterized by the general formula (I)

D-L-T (I), wherein
D is or comprises a functional moiety Z,
L is a linear linker of 10-15 atoms in length,

wherein L comprises alkyl, trans-alkylene (E —CH═CH—), and/or ether (—O—) moieties and optionally one or several methyl substituents,
T is a moiety selected from the group comprising methylamine, monofluormethylamine, difluormethylamine, trifluormethylamine, methylsulfonamide, monofluormethylsulfonamide, difluormethylsulfonamide, trifluormethylsulfonamide, azide, acetamide, monofluoracetamide, difluoracetamide, and trifluoracetamide, and hydroxyl.

In certain embodiments of the alternative of the first aspect, T is a moiety selected from the group comprising methylamine, methylsulfonamide, trifluormethylsulfonamide and hydroxyl.

The definitions of L and Z given above also apply to the alternative of the first aspect. A further alternative of the first aspect relates to a non-covalently-HaloTag-binding compound characterized by the general formula (I)

D-L-T (I), wherein
D is or comprises a functional moiety Z,
L is a linear linker of 10-15 atoms in length,

wherein L comprises alkyl, trans-alkylene, and/or ether moieties and optionally one or several methyl substituents,
T is hydroxyl.

The definitions of L and Z given above also apply to the further alternative of the first aspect.

D may comprise a linking moiety X which connects the functional moiety Z to L. In certain embodiments, X is selected from an amide moiety, an amine, a 1,2,3-triazole, an carboxylic acid ester, a sulfonamide, an ether, a thioether, a thiourea, an urea, and a carbamate.

In certain embodiments, L is a linear unbranched alkyl chain, comprising 0-4 moieties independently selected from ether and trans-alkylene. In certain embodiments, L is a linear unbranched alkyl chain, comprising 1, 2, or 3 moieties independently selected from ether and trans-alkylene.

In certain embodiments, L is an unbranched C₁₀-C₁₅ alkyl.

In certain embodiments, the compound is characterized by the one of the general formulas (II), (III), (IV), (V) or (VI),

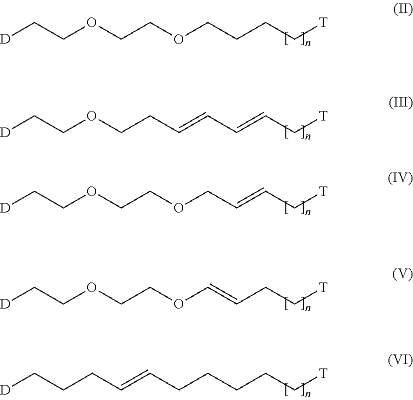

wherein n is an integer selected from 1, 2, 3, and 4. In certain embodiments, n is 1 or 2. In certain embodiments, n is 1.

In certain embodiments, the compound is characterized by the general formula (II). In certain embodiments, the compound is characterized by the general formula (III). In certain embodiments, the compound is characterized by the general formula (IV). In certain embodiments, the compound is characterized by the general formula (V). In certain embodiments, the compound is characterized by the general formula (VI).

In certain embodiments, the compound is characterized by the one of the general formulas (IIa), (IIIa), (IVa), (Va) or (VIa)

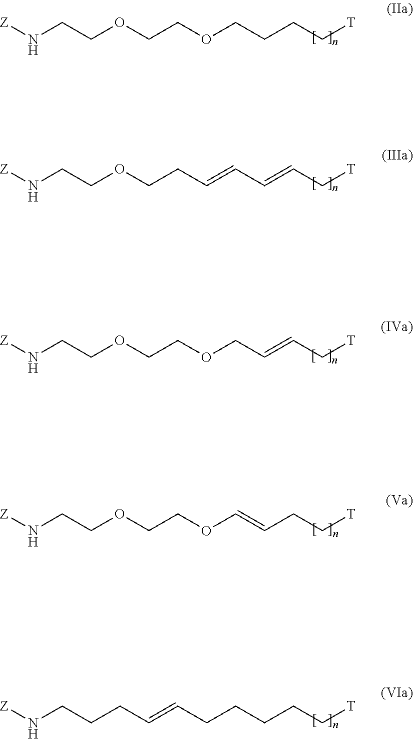

wherein n is an integer selected from 0, 1, 2, 3, and 4. In certain embodiments, n is 1 or 2.

In certain embodiments, the compound is described by (IIa), (IIIa), (IVa), (Va) or (VIa) and n is 1. In certain embodiments, the compound is described by (IIa), (IIIa), (IVa), (Va) or (VIa) and n is 2. In certain embodiments, the compound is described by (IIa), (IIIa), (IVa), (Va) or (VIa) and n is 3.

In certain embodiments, the compound is characterized by the general formula (IIa) and n is 0 or 1.

In certain embodiments, Z is a fluorophore. In certain embodiments, Z is a triarylmethane or xanthene fluorophore. In certain particular embodiments, Z is selected from a rhodamine, a silicon rhodamine, a fluorescein, a Janelia Fluor dye, an olefinic silicon rhodamine derivative with an exocyclic double bond, a cell permeable (MaP) xanthene fluorophore dye, a carbopyronine, a carbocyanine (particularly a Cy3, or a Cy5 dye), a pyrene, a Bodipy fluorophore, a coumarine, a rhodol, and an Alexa dye.

In certain embodiments, the rhodamine is selected from carboxytetramethylrhodamine

(TAMRA), tetramethylrhodamine (TMR) and the isothiocyanate derivative TRITC, sulforhodamine 101, Texas Red, and Rhodamine Red.

In certain embodiments, the silicon rhodamine is a rhodamine, wherein the central oxygen atom is replaced by SiR₂.

In certain embodiments, the fluorescein is selected from 3′,6′-dihydroxyspiro[isobenzofuran-1(3H),9′-[9H]xanthen]-3-one, fluorescein isothiocyanate (FITC) and, 6-FAM phosphoramidite. The Janelia Fluor family of molecules comprises rhodamine-type dyes having an azetidine moiety formed around the nitrogen atoms the outer rings. In certain embodiments, the Janelia Fluor dye is selected from JF646, JF635, JF585, JF549, JF525, and JF503.

In certain embodiments, the olefinic silicon rhodamine derivative with an exocyclic double bond is a structure described in WO 2019122269 A1, incorporated herein by reference.

In certain embodiments, the cell permeable (MaP) xanthene fluorophore dye is selected from MaP510, MaP555, MaP618, and MaP700 as disclosed in bioRxiv preprint doi: https://doi.org/10.1101/690867 or WO2020115286, incorporated herein by reference.

In certain embodiments, the carbocyanine is selected from tetramethylindo(di)-carbocyanine, Cy2, Cy3, Cy3B, Cy3.5, Cy5, Cy5.5, Cy7, NIR-820, ICG, Cypate, and CyTE-822.

In certain embodiments, the Bodipy fluorophore is selected from BODIPY FL, BODIPY R6G, BODIPY TMR, BODIPY 581/591, BODIPY TR, BODIPY 630/650, and BODIPY 650/665.

In certain embodiments, the Alexa dye is selected from Alexa Fluor 350, Alexa Fluor 350, Alexa Fluor 405, Alexa Fluor 430, Alexa Fluor 488, Alexa Fluor 500, Alexa Fluor 514, Alexa

Fluor 532, Alexa Fluor 546, Alexa Fluor 555, Alexa Fluor 568, Alexa Fluor 594, Alexa Fluor 610, Alexa Fluor 633, Alexa Fluor 635, Alexa Fluor 647, Alexa Fluor 660, Alexa Fluor 680, Alexa Fluor 700, Alexa Fluor 750, and Alexa Fluor 790.

In certain embodiments, the Z is of the general formula

### “Classical” Rhodamines & JF-Dyes

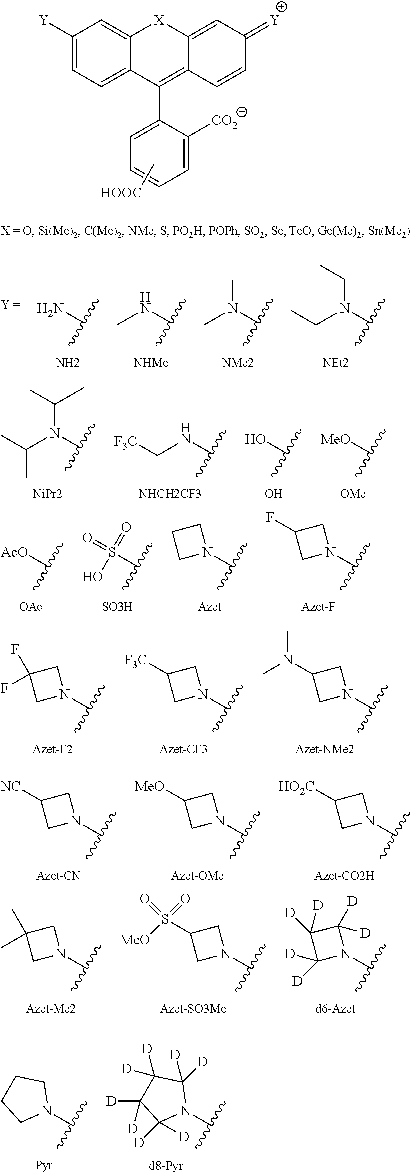

In certain embodiments, Z is of the general formula (VII) or of the general formula (VIII)

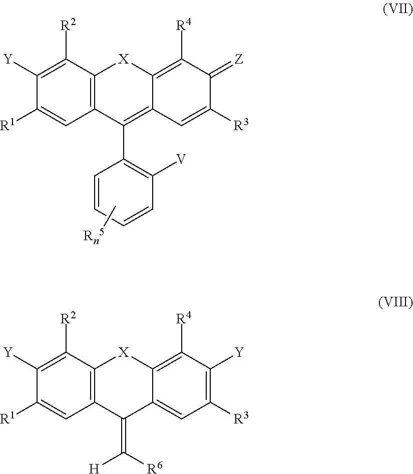

wherein
   V is selected from —C(═O)W, —CH₃, and —CH₂OH, wherein W is OH or NR^{W1}R^{W2} with R^{W1} and R^{W2} being independently selected from H, unsubstituted or amino- or hydroxy-substituted C₁-C₈ alkyl, CN, SO₂NR^{S1}R^{S2} and SO₂R^{S} with R^{S} being unsubstituted or amino- or hydroxy-substituted C₁-C₆ alkyl, and with R^{S1} and R^{S2} being independently selected from H and unsubstituted or amino- or hydroxy-substituted C₁-C₆ alkyl;
   R¹, R², R³, and R⁴ are independently selected from H and an unsubstituted or hydroxy-, amino-, halogen-, and/or carboxy-substituted hydrocarbon moiety selected from C₁-C₈ alkyl, C₃-C₈ cycloalkyl, C₁-C₄ acyl, C₇-C₁₂ alkylaryl, an unsubstituted phenyl or a phenyl substituted by any one or several of the following substituents: unsubstituted C₁-C₄ alkyl, halogen, C₁-C₄ oxyalkyl, COOH, COOR^{C}, CONR^{C}₂, with R^{C} being selected from H and unsubstituted or amino- or hydroxy-substituted C₁-C₈ alkyl;
      or R¹, R², R³, and/or R⁴ form a ring structure as described below;
   R⁵ is selected from an amine, carbonyl, ester, amide, sulfonamide, and a hydrocarbon moiety selected from C₁-C₈ alkyl, C₃-C₈ cycloalkyl, C₇-C₁₂ alkylaryl, and phenyl,
      wherein R⁵ is unsubstituted or hydroxy-, amino-, halogen-, and/or carboxy-substituted;
   n is an integer selected from 0, 1, and 2;
   R⁶ is selected from an amine, carbonyl, ester, amide, sulfonamide, and a hydrocarbon moiety selected from C₁-C₈ alkyl, C₃-C₈ cycloalkyl, C₇-C₁₂ alkylaryl, and phenyl,
      wherein R⁶ is unsubstituted or hydroxy-, amino-, halogen-, and/or carboxy-substituted;
   X is selected from O, S, Se, TeO, POR^{X}, POOR^{X}, SO₂, NR^{X}, CR^{X}₂, SIR^{X}₂, GeR^{X}₂, and SnR^{X}₂, with each R^{X} being independently selected from H and an unsubstituted or substituted (particularly unsubstituted or hydroxy-, amino-, halogen-, and/or carboxy-substituted) moiety selected from C₁-C₁₂ alkyl, C₃-C₈ cycloalkyl, C₂-C₁₂ alkenyl, C₂-C₁₂ alkynyl, C₇-C₁₂ alkylaryl, phenyl and 5- or 6-membered heteroaryl, or two RX moieties form a four-, five-, six- or seven-membered unsubstituted or amino-, hydroxy- and/or halogen substituted alkyl ring;
      particularly X is selected from O, CR^{X}₂, and SiR^{X}₂;
   Y is OH or NR^{Y1}R^{Y2},
   Z is O or N⁺R^{Y1}R^{Y2}
      with

1. RY1, and RY2 each independently selected from H, an unsubstituted or hydroxy-, amino-, halogen-, and/or carboxy-substituted hydrocarbon moiety selected from C₁-C₈ alkyl, C₃-C₈ cycloalkyl, C₁-C₄ acyl, C₇-C₁₂ alkylaryl, an unsubstituted phenyl or a phenyl substituted by any one or several of the following substituents: unsubstituted C₁-C₄ alkyl, halogen, C₁-C₄ oxyalkyl, COOH, COOR^{YC}, CONR^{YC}₂, with R^{YC} being selected from H and unsubstituted or amino- or hydroxy-substituted C₁-C₈ alkyl; or

2. R^{Y1} and R^{Y2} together are a C₃-C₆ unsubstituted or hydroxy-, amino-, halogen-, alkoxy- and/or carboxy-substituted alkyl forming a 4-7-membered ring structure with Y; or

3. one of R^{Y1} and R^{Y2}, or both R^{Y1} and R^{Y2}, together with R¹ and/or R², and/or R³ and/or R⁴, respectively, form an unsubstituted or hydroxy-, amino-, halogen-, carboxy- and/or aryl-substituted 4-7-membered alkyl or alkylene ring;

wherein Z is connected to L via a substituent selected from R¹, R², R³, R⁴, R⁵, R⁶, Y, Z and V.

A second aspect of the invention relates to a kit comprising
(a) a catalytically functional HaloTag polypeptide, particularly a wildtype HaloTag polypeptide
   wherein optionally said catalytically functional HaloTag polypeptide is attached to a polypeptide of interest;
   or
(b) an expression vector encoding said catalytically functional HaloTag polypeptide; and

a compound according to the first aspect of the invention and its embodiments.

An alternative of the second aspect of the invention relates to a kit comprising
(a) a D106 mutant HaloTag polypeptide, characterized by a polypeptide sequence wherein position D106 of the HaloTag7 sequence (position 103 designated as X in SEQ ID No 001) is exchanged for a proteinogenic amino acid different from D,
   particularly wherein the mutant HaloTag polypeptide comprises a mutation selected from D106A, D106G, and D106T,
   more particularly wherein the mutant HaloTag polypeptide comprises a D106A mutation,
or a D106 mutant HaloTag polypeptide, wherein, in a HaloTag polypeptide sequence homologous to HaloTag7, at a position homologous to D106 of HaloTag7, D is exchanged for a proteinogenic amino acid different from D,
   particularly wherein said position is changed to A, G or T,
   more particularly wherein said position is changed to A,
wherein optionally said D106 mutant HaloTag polypeptide is attached to a polypeptide of interest;
   or
(b) an expression vector encoding said D106 mutant HaloTag polypeptide; and
a compound according to the further alternative of the first aspect of the invention and its embodiments.

In other words, in the D106 mutant HaloTag polypeptide, an aspartate is exchanged for a proteinogenic amino acid different from aspartate, and this aspartate is at position 106 in the HaloTag7 polypeptide, or at the corresponding position of a homologous HaloTag polypeptide. In certain embodiments, the proteinogenic amino acid different from aspartate is an alanine, a glycine, or a threonine. In certain embodiments, the proteinogenic amino acid different from aspartate is an alanine.

A third aspect of the invention relates to a method for binding (e.g. determining the location of) a HaloTag polypeptide in a sample, the method comprising the steps of:
(a) providing an aqueous sample comprising a HaloTag polypeptide;
(b) contacting the sample with a compound comprising a fluorescent organic dye moiety according to any one of the first aspect, its alternatives and their embodiments;
(c) recording the presence and properties or location of said compound non-covalently attached to the HaloTag polypeptide in said sample by illuminating the sample with light of an excitation wavelength appropriate for exciting said fluorescent organic dye moiety, and recording light emitted from said sample, particularly at an appropriate emission wavelength λ, more particularly a λ close to a maximum of the emission spectrum of 400-800 nm,
(d) optionally repeating step c.

In certain embodiments, the binding of a HaloTag polypeptide in a sample means labelling the HaloTag polypeptide.

An alternative of the third aspect of the invention relates to a method for labelling (e.g. determining the location of) a HaloTag polypeptide in a sample, said method comprising the steps of:
providing an aqueous sample comprising a HaloTag polypeptide attached to a luciferase;
contacting the sample with a compound comprising a fluorescent organic dye moiety according to any one of the first aspect, its alternatives and their embodiments;
   contacting the sample with a luciferase substrate and recording the presence and properties or location of said compound non-covalently attached to the HaloTag polypeptide in said sample by recording light emitted from said sample, particularly at an appropriate emission wavelength λ, more particularly a λ close to a maximum of the emission spectrum of 400-800 nm;
   optionally repeating step c.

In certain embodiments, the HaloTag polypeptide is attached to a molecule of interest. In certain embodiments, the molecule of interest is a polypeptide. In certain embodiments, the HaloTag polypeptide and the polypeptide constitute a fusion polypeptide.

In certain embodiments, a first and a second HaloTag polypeptide are specifically labelled, or the location of a first and a second HaloTag polypeptide are determined,
wherein the first HaloTag polypeptide is a wildtype polypeptide and wherein the second HaloTag polypeptide is a HaloTag polypeptide mutant, wherein D106 of HaloTag7 or the analogous D of a different HaloTag, is exchanged for a different proteinogenic amino acid,
   particularly wherein the second HaloTag polypeptide comprises a mutation selected from D106A, D106G, and D106T, or the analogous mutation of D, more particularly wherein the second HaloTag polypeptide comprises a D106A mutation, or the analogous mutation of D, and
wherein a first and a second compound comprising a first fluorescent organic dye moiety and a second fluorescent organic dye, respectively, are employed,
wherein for the first compound T is a moiety selected the group comprising of methylamine, methylsulfonamide, monofluormethylsulfonamide, difluormethylsulfonamide, trifluormethylsulfonamide, azide, and acetamide, monofluoracetamide, difluoracetamide, and trifluoracetamide,
   particularly wherein T is selected from methylamine, methylsulfonamide, and trifluormethylsulfonamide,
and
wherein for the second compound T is OH, and
wherein the emission wavelength of the first fluorescent organic dye moiety is different from the emission wavelength of the second fluorescent organic dye moiety.

In certain embodiments of the third aspect of the invention or its alternative, step c is super-resolution microscopy (SRM), particularly wherein step c is point accumulation for imaging in nanoscale topography (PAINT) microscopy.

In certain embodiments, the sample comprises living cells.

A fourth aspect of the invention relates to a D106 mutant HaloTag polypeptide, wherein, in a HaloTag7 polypeptide sequence or a HaloTag polypeptide sequence homologous to HaloTag7, at position D106 of HaloTag7 or at a position homologous to D106 of HaloTag7, D is exchanged for a proteinogenic amino acid different from D,
particularly wherein said position is changed to G, A, V, I, L, C, S, T, N, or E,
more particularly wherein said position is changed to A, G or T,
even more particularly wherein said position is changed to A,

In certain embodiments, the D106 mutant HaloTag polypeptide comprises or essentially consists of a sequence selected from SEQ ID NO 001 to SEQ ID NO 007,

wherein X is selected from any proteinogenic amino acid except D,
particularly wherein X is selected from G, A, V, I, L, C, S, T, N, and E, more particularly wherein X is selected from A, G, and T, most particularly wherein X is A.

In certain embodiments, the D106 mutant HaloTag polypeptide comprises or essentially consists of SEQ ID NO 001.

A fifth aspect of the invention relates to a nucleic acid sequence encoding the D106 mutant polypeptide according to the fourth aspect of the invention and its embodiments. In certain embodiments, the wildtype HaloTag polypeptide is a sequence selected from SEQ ID NO 001-007, wherein X is D.

### Sequences

For all nucleotide sequences, (A/C) means A or C, and is designated as M in the sequence protocol according to the International Code.

Protein-sequence of HT7 (SEQ ID NO 001)
IGTGFPFDPHYVEVLGERMHYVDVGPRDGTPVLFLHGNPTSSYVWRNIIP
HVAPTHRCIAPDLIGMGKSDKPDLGYFFDDHVRFMDAFIEALGLEEVVLV
IHXWGSALGFHWAKRNPERVKGIAFMEFIRPIPTWDEWPEFARETFQAFR
TTDVGRKLIIDQNVFIEGTLPMGVVRPLTEVEMDHYREPFLNPVDREPLW
RFPNELPIAGEPANIVALVEEYMDWLHQSPVPKLLFWGTPGVLIPPAEAA
RLAKSLPNCKAVDIGPGLNLLQEDNPDLIGSEIARWLSTLEI
DNA-sequence encoding SEQ ID NO 001
(SEQ ID NO 008):
ATCGGTACTGGCTTTCCATTCGACCCCCATTATGTGGAAGTCCTGGGCGA
GCGCATGCACTACGTCGATGTTGGTCCGCGCGATGGCACCCCTGTGCTGT
TCCTGCACGGTAACCCGACCTCCTCCTACGTGTGGCGCAACATCATCCCG
CATGTTGCACCGACCCATCGCTGCATTGCTCCAGACCTGATCGGTATGGG
CAAATCCGACAAACCAGACCTGGGTTATTTCTTCGACGACCACGTCCGCT
TCATGGATGCCTTCATCGAAGCCCTGGGTCTGGAAGAGGTCGTCCTGGTC
ATTCACG(A/C)CTGGGGCTCCGCTCTGGGTTTCCACTGGGCCAAGCGCA
ATCCAGAGCGCGTCAAAGGTATTGCATTTATGGAGTTCATCCGCCCTATC
CCGACCTGGGACGAATGGCCAGAATTTGCCCGCGAGACCTTCCAGGCCTT
CCGCACCACCGACGTCGGCCGCAAGCTGATCATCGATCAGAACGTTTTTA
TCGAGGGTACGCTGCCGATGGGTGTCGTCCGCCCGCTGACTGAAGTCGAG
ATGGACCATTACCGCGAGCCGTTCCTGAATCCTGTTGACCGCGAGCCACT
GTGGCGCTTCCCAAACGAGCTGCCAATCGCCGGTGAGCCAGCGAACATCG
TCGCGCTGGTCGAAGAATACATGGACTGGCTGCACCAGTCCCCTGTCCCG
AAGCTGCTGTTCTGGGGCACCCCAGGCGTTCTGATCCCACCGGCCGAAGC
CGCTCGCCTGGCCAAAAGCCTGCCTAACTGCAAGGCTGTGGACATCGGCC
CGGGTCTGAATCTGCTGCAAGAAGACAACCCGGACCTGATCGGCAGCGAG
ATCGCGCGCTGGCTGTCTACTCTGGAGATT

The alternatives shown in the sequence protocol of SEQ ID NO 008 encode two variants of 001 for the position which are defined, in SEQ ID NO 001, as possibly filled with any amino acid. GAC encodes the wild type (D), GCC encodes the variant D106A.

Halo Tag8 (SEQ ID NO 002)
(C61S-C262S)
AEIGTGFPFDPHYVEVLGERMHYVDVGPRDGTPVLFLHGNPTSSYVWRNIIPHVAPTHRSIAPDLIGM
GKSDKPDLGYFFDDHVRFMDAFIEALGLEEVVLVIHXWGSALGFHWAKRNPERVKGIAFMEFIRPIPT
WDEWPEFARETFQAFRTTDVGRKLIIDQNVFIEGTLPMGVVRPLTEVEMDHYREPFLNPVDREPLWRF
PNELPIAGEPANIVALVEEYMDWLHQSPVPKLLFWGTPGVLIPPAEAARLAKSLPNSKAVDIGPGLNL
LQEDNPDLIGSEIARWLSTLEISG
Halo Tag9 (SEQ ID NO 003)
(Q165H-P174R, https://www.biorxiv.org/content/10.1101/
2021.04.01.438024v2)
IGTGFPFDPHYVEVLGERMHYVDVGPRDGTPVLFLHGNPTSSYVWRNIIPHVAPTHRCIAPDLIGMGK
SDKPDLGYFFDDHVRFMDAFIEALGLEEVVLVIHXWGSALGFHWAKRNPERVKGIAFMEFIRPIPTWD
EWPEFARETFQAFRTTDVGRKLIIDHNVFIEGTLRMGVVRPLTEVEMDHYREPFLNPVDREPLWRFPN
ELPIAGEPANIVALVEEYMDWLHQSPVPKLLFWGTPGVLIPPAEAARLAKSLPNCKAVDIGPGLNLLQ
EDNPDLIGSEIARWLSTLEI
DNA-sequence encoding SEQ ID NO 003 (SEQ ID NO 009):
CATCACCACCATCATCACCATCACCACCACGAAAACCTGTATTTTCAGGGCATCGGTACTGGCTTTCC
ATTCGACCCCCATTATGTGGAAGTCCTGGGCGAGCGCATGCACTACGTCGATGTTGGTCCGCGCGATG
GCACCCCTGTGCTGTTCCTGCACGGTAACCCGACCTCCTCCTACGTGTGGCGCAACATCATCCCGCAT
GTTGCACCGACCCATCGCTGCATTGCTCCAGACCTGATCGGTATGGGCAAATCCGACAAACCAGACCT
GGGTTATTTCTTCGACGACCACGTCCGCTTCATGGATGCCTTCATCGAAGCCCTGGGTCTGGAAGAGG
TCGTCCTGGTCATTCACG(A/C)CTGGGGCTCCGCTCTGGGTTTCCACTGGGCCAAGCGCAATCCAGA
GCGCGTCAAAGGTATTGCATTTATGGAGTTCATCCGCCCTATCCCGACCTGGGACGAATGGCCAGAAT
TTGCCCGCGAGACCTTCCAGGCCTTCCGCACCACCGACGTCGGCCGCAAGCTGATCATCGATCATAAC
GTTTTTATCGAGGGTACGCTGCTGATGGGTGTCGTCCGCCCGCTGACTGAAGTCGAGATGGACCATTA
CCGCGAGCCGTTCCTGAATCCTGTTGACCGCGAGCCACTGTGGCGCTTCCCAAACGAGCTGCCAATCG
CCGGTGAGCCAGCGAACATCGTCGCGCTGGTCGAAGAATACATGGACTGGCTGCACCAGTCCCCTGTC
CCGAAGCTGCTGTTCTGGGGCACCCCAGGCGTTCTGATCCCACCGGCCGAAGCCGCTCGCCTGGCCAA
AAGCCTGCCTAACTGCAAGGCTGTGGACATCGGCCCGGGTCTGAATCTGCTGCAAGAAGACAACCCGG
ACCTGATCGGCAGCGAGATCGCGCGCTGGCTGTCTACTCTGGAGATT
HOB (SEQ ID NO 004)
(Halo-based oligonucleotide binder, E154K-E158K-T166K-D167K)
IGTGFPFDPHYVEVLGERMHYVDVGPRDGTPVLFLHGNPTSSYVWRNIIPHVAPTHRCIAPDLIGMGK
SDKPDLGYFFDDHVRFMDAFIEALGLEEVVLVIHXWGSALGFHWAKRNPERVKGIAFMEFIRPIPTWD
EWPKFARKTFQAFRTKKVGRKLIIDQNVFIEGTLPMGVVRPLTEVEMDHYREPFLNPVDREPLWRFPN
ELPIAGEPANIVALVEEYMDWLHQSPVPKLLFWGTPGVLIPPAEAARLAKSLPNCKAVDIGPGLNLLQ
EDNPDLIGSEIARWLSTLEISG
DNA-sequence encoding SEQ ID NO 004 (SEQ ID NO 010):
ATGCATCACCACCATCATCACCATCACCACCACGAAAACCTGTATTTTCAGGGCATTGGCACCGGTTT
TCCGTTTGATCCGCATTATGTTGAAGTTCTGGGTGAACGTATGCATTATGTGGATGTTGGTCCGCGTG
ATGGTACACCGGTTCTGTTTCTGCATGGTAATCCGACCAGCAGCTATGTTTGGCGTAACATTATTCCG
CATGTTGCACCGACCCATCGTTGTATTGCACCGGATCTGATTGGTATGGGTAAAAGCGATAAACCTGA
TCTGGGCTATTTTTTCGATGATCATGTGCGTTTTATGGACGCCTTTATTGAAGCACTGGGTCTGGAAG
AAGTTGTGCTGGTTATTCATG(A/C)TTGGGGTAGCGCACTGGGTTTTCATTGGGCAAAACGTAATCC
GGAACGTGTTAAAGGTATTGCCTTTATGGAATTTATTCGTCCGATTCCGACCTGGGATGAATGGCCGA
AATTTGCACGTAAAACCTTTCAGGCATTTCGCACCAAAAAAGTTGGTCGCAAACTGATTATTGACCAG
AACGTTTTTATCGAAGGCACCCTGCCGATGGGTGTTGTTCGTCCGCTGACCGAAGTTGAAATGGATCA
TTATCGTGAACCGTTTCTGAATCCGGTTGATCGCGAACCGCTGTGGCGTTTTCCGAATGAACTGCCGA
TTGCCGGTGAACCTGCAAATATTGTTGCACTGGTTGAAGAGTATATGGATTGGCTGCATCAGAGTCCG
GTTCCGAAACTGCTGTTTTGGGGCACACCGGGTGTTCTGATTCCGCCTGCAGAAGCAGCACGTCTGGC
AAAAAGCCTGCCGAATTGTAAAGCAGTTGATATTGGTCCGGGTCTGAATCTGCTGCAAGAAGATAATC
CAGATCTGATCGGTAGTGAAATTGCACGTTGGCTGAGCACCCTGGAAATTAGCGGCTAA
Circular permuted Halo Tag variants
cpHalo 141/144 (SEQ ID NO 005)
GGGFARETFQAFRTTDVGRKLIIDQNVFIEGTLPMGVVRPLTEVEMDHYREPFLNPVDREPLWRFPNE
LPIAGEPANIVALVEEYMDWLHQSPVPKLLFWGTPGVLIPPAEAARLAKSLPNCKAVDIGPGLNLLQE
DNPDLIGSEIARWLSTLEIGGTGGSGGTGGSGGSIGTGFPFDPHYVEVLGERMHYVDVGPRDGTPVLF
LHGNPTSSYVWRNIIPHVAPTHRCIAPDLIGMGKSDKPDLGYFFDDHVRFMDAFIEALGLEEVVLVIH
XWGSALGFHWAKRNPERVKGIAFMEFIRPIPTWDEW
DNA-sequence encoding SEQ ID NO 005 (SEQ ID NO 011):
CATCACCACCATCATCACCATCACCACCACGAAAACCTGTATTTTCAGGGCGGTGGCTTTGCCCGCGA
GACCTTCCAGGCCTTCCGCACCACCGACGTCGGCCGCAAGCTGATCATCGATCAGAACGTTTTTATCG
AGGGTACGCTGCCGATGGGTGTCGTCCGCCCGCTGACTGAAGTCGAGATGGACCATTACCGCGAGCCG
TTCCTGAATCCTGTTGACCGCGAGCCACTGTGGCGCTTCCCAAACGAGCTGCCAATCGCCGGTGAGCC
AGCGAACATCGTCGCGCTGGTCGAAGAATACATGGACTGGCTGCACCAGTCCCCTGTCCCGAAGCTGC
TGTTCTGGGGCACCCCAGGCGTTCTGATCCCACCGGCCGAAGCCGCTCGCCTGGCCAAAAGCCTGCCT
AACTGCAAGGCTGTGGACATCGGCCCGGGTCTGAATCTGCTGCAAGAAGACAACCCGGACCTGATCGG
CAGCGAGATCGCGCGCTGGCTGTCTACTCTGGAGATTGGTGGCACCGGTGGCTCTGGCGGTACCGGTG
GCTCTGGCGGTTCTATCGGTACTGGCTTTCCATTCGACCCCCATTATGTGGAAGTCCTGGGCGAGCGC
ATGCACTACGTCGATGTTGGTCCGCGCGATGGCACCCCTGTGCTGTTCCTGCACGGTAACCCGACCTC
CTCCTACGTGTGGCGCAACATCATCCCGCATGTTGCACCGACCCATCGCTGCATTGCTCCAGACCTGA
TCGGTATGGGCAAATCCGACAAACCAGACCTGGGTTATTTCTTCGACGACCACGTCCGCTTCATGGAT
GCCTTCATCGAAGCCCTGGGTCTGGAAGAGGTCGTCCTGGTCATTCACG(A/C)CTGGGGCTCCGCTC
TGGGTTTCCACTGGGCCAAGCGCAATCCAGAGCGCGTCAAAGGTATTGCATTTATGGAGTTCATCCGC
CCTATCCCGACCTGGGACGAATGG
cpHalo 153/156 (SEQ ID NO 006)
GGGDVGRKLIIDQNVFIEGTLPMGVVRPLTEVEMDHYREPFLNPVDREPLWRFPNELPIAGEPANIVA
LVEEYMDWLHQSPVPKLLFWGTPGVLIPPAEAARLAKSLPNCKAVDIGPGLNLLQEDNPDLIGSEIAR
WLSTLEIGGTGGSGGTGGSGGSIGTGFPFDPHYVEVLGERMHYVDVGPRDGTPVLFLHGNPTSSYVWR
NIIPHVAPTHRCIAPDLIGMGKSDKPDLGYFFDDHVRFMDAFIEALGLEEVVLVIHXWGSALGFHWAK
RNPERVKGIAFMEFIRPIPTWDEWPEFARETFQAFR
DNA-sequence encoding SEQ ID NO 006 (SEQ ID NO 012):
CATCACCACCATCATCACCATCACCACCACGAAAACCTGTATTTTCAGGGCGGTGGCGACGTCGGCCG
CAAGCTGATCATCGATCAGAACGTTTTTATCGAGGGTACGCTGCCGATGGGTGTCGTCCGCCCGCTGA
CTGAAGTCGAGATGGACCATTACCGCGAGCCGTTCCTGAATCCTGTTGACCGCGAGCCACTGTGGCGC
TTCCCAAACGAGCTGCCAATCGCCGGTGAGCCAGCGAACATCGTCGCGCTGGTCGAAGAATACATGGA
CTGGCTGCACCAGTCCCCTGTCCCGAAGCTGCTGTTCTGGGGCACCCCAGGCGTTCTGATCCCACCGG
CCGAAGCCGCTCGCCTGGCCAAAAGCCTGCCTAACTGCAAGGCTGTGGACATCGGCCCGGGTCTGAAT
CTGCTGCAAGAAGACAACCCGGACCTGATCGGCAGCGAGATCGCGCGCTGGCTGTCTACTCTGGAGAT
TGGTGGCACCGGTGGCTCTGGCGGTACCGGTGGCTCTGGCGGTTCTATCGGTACTGGCTTTCCATTCG
ACCCCCATTATGTGGAAGTCCTGGGCGAGCGCATGCACTACGTCGATGTTGGTCCGCGCGATGGCACC
CCTGTGCTGTTCCTGCACGGTAACCCGACCTCCTCCTACGTGTGGCGCAACATCATCCCGCATGTTGC
ACCGACCCATCGCTGCATTGCTCCAGACCTGATCGGTATGGGCAAATCCGACAAACCAGACCTGGGTT
ATTTCTTCGACGACCACGTCCGCTTCATGGATGCCTTCATCGAAGCCCTGGGTCTGGAAGAGGTCGTC
CTGGTCATTCACG(A/C)CTGGGGCTCCGCTCTGGGTTTCCACTGGGCCAAGCGCAATCCAGAGCGCG
TCAAAGGTATTGCATTTATGGAGTTCATCCGCCCTATCCCGACCTGGGACGAATGGCCAGAATTTGCC
CGCGAGACCTTCCAGGCCTTCCGC
cpHalo 154/156 (SEQ ID NO 007)
GGGDVGRKLIIDQNVFIEGTLPMGVVRPLTEVEMDHYREPFLNPVDREPLWRFPNELPIAGEPANIVA
LVEEYMDWLHQSPVPKLLFWGTPGVLIPPAEAARLAKSLPNCKAVDIGPGLNLLQEDNPDLIGSEIAR
WLSTLEIGGTGGSGGTGGSGGSIGTGFPFDPHYVEVLGERMHYVDVGPRDGTPVLFLHGNPTSSYVWR
NIIPHVAPTHRCIAPDLIGMGKSDKPDLGYFFDDHVRFMDAFIEALGLEEVVLVIHXWGSALGFHWAK
RNPERVKGIAFMEFIRPIPTWDEWPEFARETFQAFRT
DNA-sequence encoding SEQ ID NO 007 (SEQ ID NO 013):
CATCACCACCATCATCACCATCACCACCACGAAAACCTGTATTTTCAGGGCGGTGGCGACGTCGGCCG
CAAGCTGATCATCGATCAGAACGTTTTTATCGAGGGTACGCTGCCGATGGGTGTCGTCCGCCCGCTGA
CTGAAGTCGAGATGGACCATTACCGCGAGCCGTTCCTGAATCCTGTTGACCGCGAGCCACTGTGGCGC
TTCCCAAACGAGCTGCCAATCGCCGGTGAGCCAGCGAACATCGTCGCGCTGGTCGAAGAATACATGGA
CTGGCTGCACCAGTCCCCTGTCCCGAAGCTGCTGTTCTGGGGCACCCCAGGCGTTCTGATCCCACCGG
CCGAAGCCGCTCGCCTGGCCAAAAGCCTGCCTAACTGCAAGGCTGTGGACATCGGCCCGGGTCTGAAT
CTGCTGCAAGAAGACAACCCGGACCTGATCGGCAGCGAGATCGCGCGCTGGCTGTCTACTCTGGAGAT
TGGTGGCACCGGTGGCTCTGGCGGTACCGGTGGCTCTGGCGGTTCTATCGGTACTGGCTTTCCATTCG
ACCCCCATTATGTGGAAGTCCTGGGCGAGCGCATGCACTACGTCGATGTTGGTCCGCGCGATGGCACC
CCTGTGCTGTTCCTGCACGGTAACCCGACCTCCTCCTACGTGTGGCGCAACATCATCCCGCATGTTGC
ACCGACCCATCGCTGCATTGCTCCAGACCTGATCGGTATGGGCAAATCCGACAAACCAGACCTGGGTT
ATTTCTTCGACGACCACGTCCGCTTCATGGATGCCTTCATCGAAGCCCTGGGTCTGGAAGAGGTCGTC
CTGGTCATTCACG(A/C)CTGGGGCTCCGCTCTGGGTTTCCACTGGGCCAAGCGCAATCCAGAGCGCG
TCAAAGGTATTGCATTTATGGAGTTCATCCGCCCTATCCCGACCTGGGACGAATGGCCAGAATTTGCC
CGCGAGACCTTCCAGGCCTTCCGCACC

### DESCRIPTION OF FIGURES

FIG. 1 shows binding affinities of HT7 and TMR-nrHTL initial candidates. A. Chemical structures of nrHTL TMR-28 and TMR-23. B. Comparison of experimental and calculated binding preferences among the nrHTL candidates. C. Titration curves of different TMR-nrHTL with HT7. Binding was measured by fluorescence polarization in techn. triplicates. Avg. data and SD were normalized to lower and higher polarization values and fitted with equation (2). K_{D} given as concentration at half-maximal fluorescence polarization with a Hill coefficient n=1. Mean K_{D}-values and SD from at least 3 independent experiments (xₙ≤3) are presented. MSA—methylsulfonamide, MA—methylamine.

FIG. 2 shows binding affinities and labeling of TMR- and SiR-nrHTL second candidates. A. Chemical structures of nrHTL2 Dye-24. B. Binding affinities of different nrHTLs. Binding was measured by FP in techn. triplicates. Avg. data were fitted with equation (2) yielding KD as the concentration at half-maximal fluorescence polarization with n=1 (Hill coefficient). Mean KD-values and SD from at least 3 independent experiments (xn≤3) are given. C. Reversibility of the nrHTL interaction to HT7. Coomassie-stained SDS-PAGE and in-gel fluorescence of HT7 in excess of ligand (5:20 μM) after 30 min incubation at 37° C.

FIG. 3 shows binding affinity of various rhodamine-based dyes modified with nrHTL1 (23). A. Chemical structures of 6-carboxy-Atto565 and -CPy B. Representative selection of titration curves comparing different nrHTL1 Dye-23 binding to HT7. Avg. data and SD from techn. triplicates were normalized to lower and higher polarization values and fitted with equation (2). C. Binding affinities and Hill coefficient determination. KD given as concentration at half-maximal fluorescence polarization from the titration curves presented in B. The Hill coefficient n was extracted as the slope of the regression curves. Mean values and SD from min. three independent experiments (xn≤3) are given.

FIG. 4 shows binding kinetics of nrHTL to HT7. A. Cartoon representation of binding parameters involved in nrHTL binding to HT7 important for PAINT microscopy: τb—bright time, τd—dark time. Lower panel adopted from Jungmann et al. Nat. Meth. (2016). B. Stopped-flow binding kinetics of TMR-24 and −28 to HT7. FP was tracked over time, data from 10 techn. replicates were averaged, normalized to higher polarization values and fitted with equation (3). Magnification of first second shown in the gray window besides. C. Binding kinetics of TMR- and SiR-23/-24 to HT7 obtain similarly. D. Binding properties of MSA nrHTL to HT7. k1 extracted from kinetic measurements, errors are represented by the regression standard error. k−1 were calculated with eq. (4) from experimental data and errors were determined by standard error propagation.

FIG. 5 shows nrHTL ligands bound HT7 by polar interactions with Asp106. A. Schematic representation of proposed nrHTL binding mode. Reversible binding of rhodamine nrHTL and magnification to the HT7 active centre. Chemical structures of the side chain of the amino acids N41, D106 and W107 together with the terminal segment of nrHTL2 TMR-24 are represented. B. Binding affinities of nrHTL1 3 with HT7/dHTD106A as well as TMR-33 with dHTD106A. C. Bar graphs of log KD-values presented in B for nrHTL1-3. Binding was measured by FP in techn. triplicates. Avg. data were fitted with equation (2) whereat KD given as concentration at half-maximal fluorescence polarization with n=1 (Hill coefficient). Mean KD-values and SD from at least 3 independent experiments (xn≤3) are given. MSA—methylsulfonamide. MA—methylamine.

FIG. 6 shows In-silico evaluation of the nrHTL moieties protonation state. A. Representation of the protonation equilibrium of amine and sulfonamide functions leading to the evaluation of pKa1 and pKa2. Less abundant state under physiological conditions is presented in gray. B. In-silico pKa-calculation of different secondary amine and (sulfon)amide residues together with the resulting estimated charge under physiological conditions. Calculations were performed by using Schrödinger tool Epic. +: positive, o: neutral, −: negative.

FIG. 7 shows pH-Dependent binding of TMR-28 to HT7.A. Chemical structure of TMR-28 below pH 10. B. Affinities and binding kinetics measured for TMR 28 and HT7 at different pH. C. pH-Dependent titration of TMR-28 binding to HT7. Fluorescence polarization was measured in techn. triplicates, the data were normalized to lower and higher polarization values and fitted with equation (2). KD given as concentration at half-maximal fluorescence polarization with regression standard error. D. pH-Dependent binding kinetics of TMR-28 to HT7. Data from 3 techn. replicates were averaged, normalized to higher polarization values and fitted with equation (3) yielding k1. Errors are represented as the regression standard error.

FIG. 8 shows spirolactone equilibrium & HT7-induced SiR fluorescence turn-on of nrHTLs. A. Water/dioxane titration of SIR-23, -28 and -HTL. Normalized absorbance at 646 nm of SiR derivatives in water-dioxane mixtures (v/v, 10/90-80/20) as a function of the dielectric constant ER. Mean values from 3 techn. replicates were fitted with equation (5), error bars presented by the SD. B. Mean D50-values and SD of the different HTLs from 3 independent experiments (xn=3). C. Fluorescence emission spectra of SiR-23 in absence (dashed line) and presence (straight line) of HT7. Avg. spectra from 3 techn. replicates, normalized to SIR-HTL emission spectra in presence of HT7. D. Fluorescence turn-on of SiR 23, 24, 28 and -HTL was extracted from the data shown in C. based on the emission maxima at 670 nm. The errors were determined by standard error propagation. 23—C4-MSA, 24—C5-MSA, 28—C5-MA.

FIG. 9 shows colocalization of HT7 stained with nrHTL SiR-28 and SNAP-TMR in fixed cells.Sum of 8 confocal z-stacks (z=2 nm) of chemically arrested U-2 OS cells expressing HaloTag7-SNAP-NLS stained with 500 nM SiR-28 and TMR-BG. Images were taken under no-washed conditions with the following excitation lasers: TMR: 560 nm (6.0%), SIR: 633 nm (3.0%). Scale bar: 50 μm.

FIG. 10 shows cell permeability of nrHTL probes. Confocal fluorescence imaging of SiR-(A) and TMR-23 (B) staining of HT7-SNAP-NLS in fixed and live U-2 OS cell's nuclei. Images were taken under no-washed conditions with the following excitation lasers: SiR: 633 nm (3.0%), TMR: 560 nm (fixed: 6.0%, live: 12%). Scale bars: 50 μm. 23—C4-MSA.

FIG. 11 shows improving the fluorogenic effect of nrHTL1 (23). A. General chemical structure of MaP/JF Dyes: X=O (rhodamine), C(CH₃)2 (carbopyronine) or Si(CH₃)2 (silicon rhodamine). Z=—NSO2—N(CH₃)2. JF525, JF585, JF615: R=R′=F. JF635: R=H, R′=F. JF656: R =R′=H. B. Selection of emission spectra of nrHTL1 Dye-23 in absence (dashed line) and presence (straight line) of HT7. Avg. data from techn. triplicates were normalized to the respective HTL emission spectra. C. Fluorescence turn-on upon binding to HT7 and HT8 shown as bar graphs. It was calculated based on the emission maxima, respectively. Intensity from three techn. replicates were averaged and the errors were determined by standard error propagation. 23: C4-MSA.

FIG. 12 shows biochemical characterization of the fluorogenic nrHTLs binding to HaloTags. A. Binding kinetics comparison of nrHTL1 SIR-23 to HT7 and HT8, measured by FP under stopped flow-conditions. B. Summarizing table of the biochemical characterization of SiR-, JF585- and JF635-23 with HT7 and HT8. KD given as concentration at half-maximal fluorescence polarization from titration curves. Avg. values from at least three individual experiments (xn≥3) are given and errors are represented as SD. Kinetic traces from A. were fitted with equation (3) delivering k1, avg. value and standard regression error from 8 techn. replicates given. k−1 were calculated from experimental data using equation (4) and errors were determined by standard error propagation. 23: C4-MSA.

FIG. 13 shows in-vitro characterization of the potential nrHTLs 25 and 26. A. Chemical structure of TMR-25 and -26. B. Titration curves of TMR-25 and -26 with HT7 in comparison to TMR-23. Avg. data of techn. triplicates and SD were normalized to higher polarization and fitted with equation (2). C. Stopped-flow binding kinetics of TMR-23 versus TMR-25 to HT7. FP data from 8 techn. replicates were normalized to higher polarization and fitted with equation (3). D. 3D-model of functional groups used as binding motifs for nrHTL 23-26 indicating progressing planarization. Adopted from Shainyan et al. Chem Rev (2013). E-G. Crystal structure of TMR-25-complexed HT7 in comparison to TMR-23. The protein structure is represented as pale cyan cartoon while ligand and interacting residues are represented as indicated colored sticks, respectively. Straight dashed gray lines illustrate putative hydrogen bonds between protein and ligand (in Å) whereat gray curves show the angle (in °) between two ligand atoms. All-atom RMSD was calculated manually using the pymol software. H. Summarizing table of the TMR- and SiR-25 and 26 interaction with HT7. Avg. KD-values and SD from at least three individual experiments shown (xn≥3). k1 and regression standard error extracted from C. k−1 were calculated from experimental data using eq. (4), errors were determined by standard error propagation. I. Non-covalent binding of TMR-25 to HT7. In-vitro protein labeling at 37° C. followed by SDS-PAGE and in-gel fluorescence imaging in comparison to TMR-HTL.

FIG. 14 shows that Triflamide nrHTL4 improves various dyes fluorogenicity upon HT binding. A. Comparison of fluorescence emission spectra of SIR-25 and SiR-HTL in presence/absence (straight/dashed lines) of HT7. B. Fluorescence emission increase upon MaP555-, JF585- and SiR-25 binding to HT7 and HT8. Intensity at emission maxima from techn. triplicates were averaged, data are shown as bar graphs and the error bars are represented by standard error propagation. 25: C4-F3MSA.

FIG. 15 shows Characterization of cpHaloTag combination with nrHTLs. A. Structural representation of the circular permutation sites probed in HaloTag7 (PDB: 6Y7A). HaloTag7 is represented as gray cartoon. The circular permutation sites (i.e. new termini) are highlighted by the αC atom represented as indicated colored spheres, respectively. The TMR substrate bound to the protein is represented as green sticks. B. Titration curves comparison of TMR-24 with HT7 and cp-candidates. FP data from techn. triplicates were averaged, normalized to lower and higher polarized values and fitted with eq. (2). C. Stopped-flow binding kinetic comparison between HT7 and cpHT7-154/156 to TMR-24. Avg. FP data from 10 techn. replicates were fitted with equation (3). D. Live-cell staining of U-2 OS cells transiently transfected with H2B-cpHaloTag154/156-T2A-eGFP with 500 nM SiR-24 in comparison to SiR-HTL. Images were acquired under no-wash confocal conditions with a 488 nm (8.0%, eGFP) and a 633 nm (1.0%, SiR) laser. Scale bars: 10 μm. E. Summarizing table of the binding properties of TMR 24 to HT7 and different cpHaloTag variants. Avg. KD-value and SD from three individual FP titration experiments given. k1-value extracted from C. given with regression standard error. k−1 were calculated from experimental data with eq. (4) and errors were calculated by standard error propagation. 24: C5-MSA.

FIG. 16 shows Heat map representing potential TMR-nrHTL binding affinity to HT7 variants.Binding affinities (KD) were obtained from titration curves done by fluorescence polarization. Avg. data from techn. triplicates were fitted with equation (2). KD yielded from titration curves as concentration at half-maximal fluorescence polarization with a Hill coefficient equals 1. The dataset was colored based on the KD-value between red (highest value) and green (lowest values) with a midpoint of 20% of the population (yellow). 24: C5-MSA, 27: C4-N3, 28: C5-MA, 30: C4-OH and 33: C6.

FIG. 17 shows Characterization of the dHTL/dHT7D106A system. A. Chemical structure of potential dHTL TMR-29 to -32. B. Heat-map representation of KD-values determined by FP between TMR- or SiR-29 to 31 and HT7 or dHT7D106A. C. Representative titration curves showing binding of TMR-29 to -32 to dHT7D106A. FP data from techn. triplicates were averaged and fitted with eq. (2). D. Stopped-flow kinetic traces for dHT7D106A ligand candidates. Data from 10 techn. replicates were averaged and fitted with eq. (3). E. Fluorescence turn-on of SiR-30 and -31 upon binding to dHT7D106A F. Summary of binding properties for TMR- 30, -31 and SiR-30. Mean KD-value and SD from three individual experiments shown. k1, value extracted from D. and standard regression error given. k1 were calculated from experimental data using eq. (4) and errors were determined by standard error propagation. G. Confocal live-cell, no-wash staining of U-2 OS transiently expressing H2B-dHT7D106A-T2A-eGFP with 500 nM TMR-31 or SiR 30. The following excitation lasers were used: TMR: 550 nm (3.0%), SiR 633 nm (1.0%). Dashed white lines indicate surrounding, untransfected cell's nuclei from bright-field images. Scale bars: 10 μm. 29: C3-OH, 30: C4—OH, 31: C5—OH, 32: C4-OMe.

The following pages give the examples for the present specification:

### EXAMPLE 1

### Ligand Development and Synthesis

Non-reactive HaloTag7 Ligands (nrHTLs) were designed based on the chemical structure of TMR modified with the chloroalkane HaloTag7 substrate (TMR-PEG₂-C₆-Cl), usually dubbed as HaloTag Ligand (HTL).

### Example 2

### Characterization of Non-Reactive HT7 Probes for PAINT Microscopy

Identification of the Best nrHTL Candidates Through In-Vitro Characterization

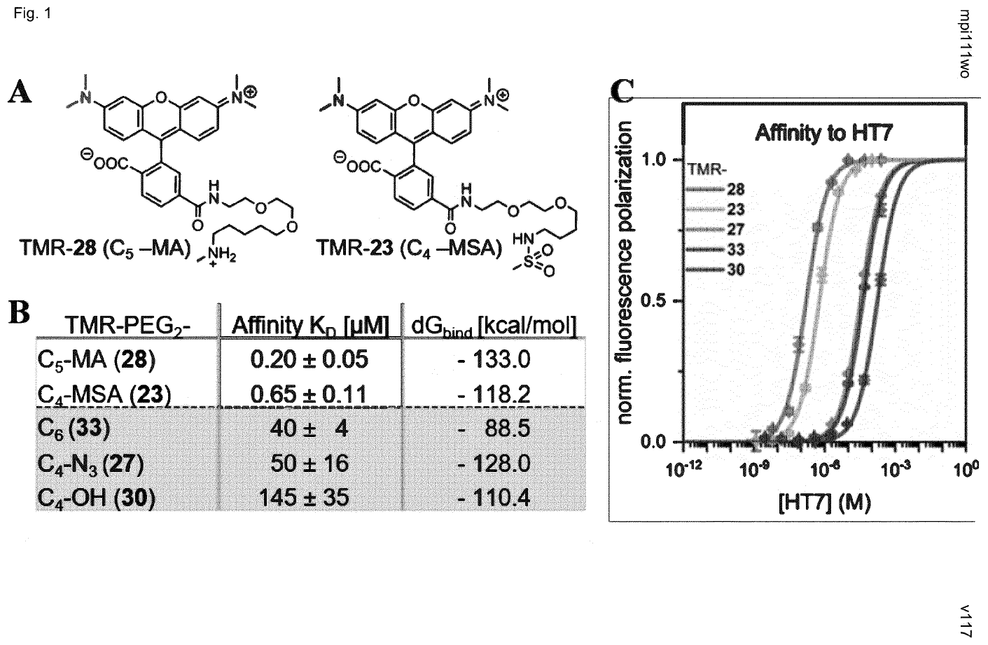

PAINT-microscopy experiments are usually performed with DNA yet more recently protein/protein and protein/ligand-pairs interacting were employed. The latter systems utilize affinities of 0.3 to 1.4 μM, which turned out to be instrumental for achieving high-resolution images. To prove the concept of nrHTL as PAINT probes the binding affinities (K_{D}) to HT7 were measured via fluorescence polarization for a selection of identified and synthesized potential nrHTL (FIG. 1).

The methylsulfonamide—(MSA) and methylamine-bearing (MA) compounds (TMR-23 and -28) presented sub-μM binding affinities, while the remaining compounds (TMR-27, -30 and -33) showed K_{D} in the moderate mM range (FIG. 1B, C). The obtained binding affinities makes it clear, that TMR-23 and -28 (FIG. 1A) can be considered as good nrHTL candidates.

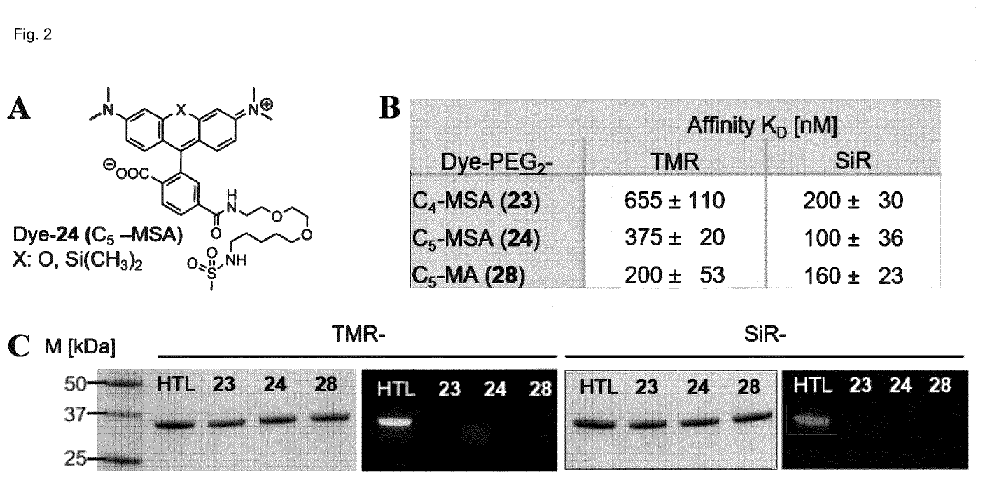

The two best nrHTLs candidates (TMR-23 and TMR-28) show K_{D} differences by a factor of 3.3. However, because both the binding moiety (MSA vs. MA) as well as the terminal linker length (C₄ vs. C₅) differ between them, preventing a clear structure-function analysis. Therefore, a comparable TMR-24 (C₅-MSA, FIG. 2A) was synthesized, together with SiR versions of theses three nrHTL candidates. Their binding affinities were characterized by measuring the K_{D} by FP (FIG. 2B). It was discovered, that the use of a longer C₅-linker (TMR-24) enhanced the binding affinity in comparison to TMR-23 by a factor of ˜2. Affinity measurements including SiR compounds confirmed this trend and overall showed higher K_{D}-values.

The non-covalent nature of the potential nrHTLs was verified by protein labeling followed by SDS-PAGE and fluorescence scan of the gel (FIG. 2C). Labeling for 30 minutes and 37° C. did not lead to a covalent linkage between TMR- or SiR-23, 24 nor 28 in contrast to the HTL substrates that showed intense fluorescence signals received from the respective protein band. Long-term experiments (>48 h) at room-temperature were carried out and exhibit the same results. From here on, fluorophore-conjugated ligand 23, 24 and 28 are also titled as non-reactive HaloTag Ligands 1 to 3 (nrHTL₁ to nrHTL₃).

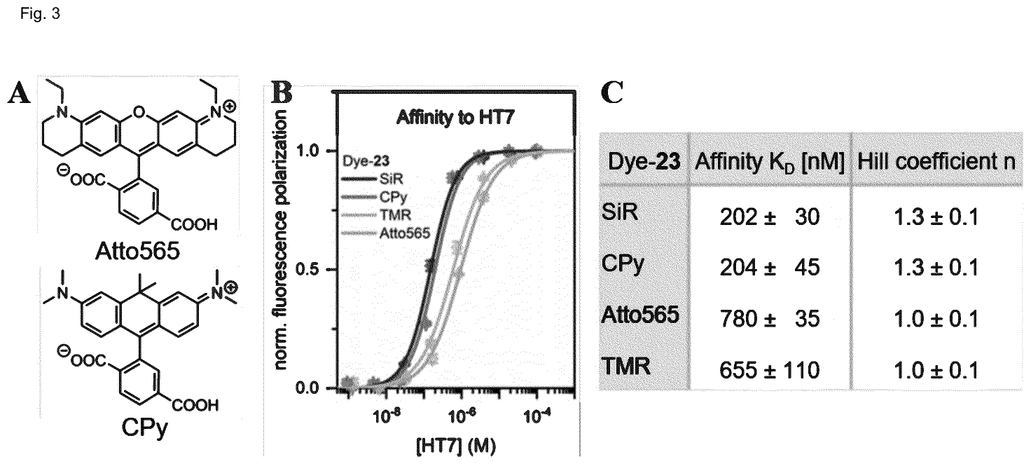

Observing the beneficial influence of SiR in contrast to TMR on the nrHTL affinity questions the effect of the fluorophore on the binding process. Therefore, dissociation constants K_{D} were measured for nrHTL₁ (23) coupled to CPy and Atto565 fluorophores (FIG. 3A), revealing similar K_{D}-values for SiR and CPy, likely due to their strong structural and electronical similarities (FIG. 3B, C). On the other hand, a lower binding affinity was measured with Atto565-23 which could be imputed to the large modification of the xanthene ring, possibly inducing steric hindrances with the HT protein, together with a more electron-rich central atom.

Besides the effect on the binding affinity, also a significant difference in the Hill coefficient between different dyes was recognized: rhodamine-derived dyes (X=O) exhibit the expected Hill coefficient of 1, matching the non-cooperative, one-site binding mode expected for HT7. In contrast, the data obtained for nrHTL₁ modified CPy and SiR dyes is more accurately represented by n=1.3 (FIG. 3C). The underlying mechanism causing this difference needs further assessment, however it does not strongly affect the K_{D}-value determination.

Kinetics of nrHTL Binding to HT7

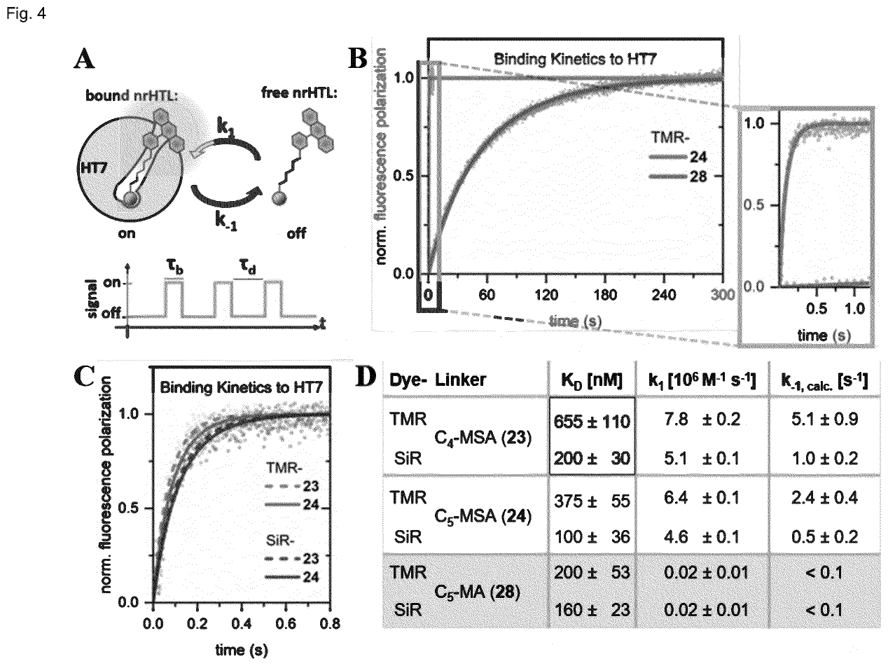

PAINT imaging is allowed by an adequate time presence of one fluorophore on a given cellular structure. Within this time span it is required to collect sufficient photons to precisely locate a single molecule at a reasonable frame-rate. Conversely, the maximal imaging speed is limited by the (un-)binding kinetic parameters. Affinities (K_{D}) are characterized by kinetic constants of binding k₁ (on-rate) and unbinding k₋₁ (off-rate) such that the quotient of both result in K_{D}. In PAINT-microscopy, k₋₁ is particularly important because it defines the time presence of the fluorophore on the target (bright-time τ=1/k₋₁). Above that, k₁ largely influences the imaging speed at a given probe concentration c (dark-time τ_{d}=k₁·c, FIG. 4A).

Therefore, the binding kinetics of nrHTL TMR-24 and TMR-28 to HT7 were measured by fluorescence polarization under stopped-flow conditions (see FIG. 4B). Surprisingly, a significant difference in the binding kinetics between the two classes of binding moieties (methylsulfonamides vs. methylamines) was observed. TMR-28 (MA) shows a binding kinetics to HT7 completed in the 3 to 5 min range, whereas the methylsulfonamide ligand

TMR-24 binding reached completion within 0.5 s under similar conditions. This resulted in a significant difference of about 4 orders of magnitude in binding speed compared to TMR-28, whilst having similar K_{D}-values. Furthermore, the impact of the linker length (C₄ VS. C₅) and the fluorophore (TMR vs. SiR) on the binding kinetics of MSA ligands was investigated (FIG. 4C) with the goal to elucidate the most adequate PAINT probes. In combination with the dissociation constants K_{D} determined in the previous chapter, the off-rates k₋₁ were calculated (FIG. 4D).

C₄-MSA nrHTLs present an overall lower affinity for HT7 as compared to their corresponding C₅-MSA derivatives. Noticeably, this difference could be mostly attributed to an off-rate difference of overall two-fold whereat both ligands yield almost equivalent on-rates. Those findings are consistent for TMR and SiR probes. For the purpose of PAINT-microscopy, the optimal k₋₁-values were reported to be above 1 s⁻¹ allowing high camera frame-rates without compromising S/N. Besides, the dark-time τ_{d} is of utmost importance for fast image acquisition at a preferable low probe concentration, wherefore k₁-values in the range of 10⁶ to 10⁷ M⁻¹ s⁻¹ are favourable. Concludingly, nrHTL₁ 23 in combination with both dyes tested represents the most promising PAINT probe.

In contrast, nrHTL₃ 28 revealed binding kinetics that are not suitable for this particular purpose. Nevertheless, the striking difference in binding speed observed will be the object of the following investigations aiming to identify the binding mode of the neo-developed nrHTL.

Functional and Structural Investigations on the nrHTL Binding Mode

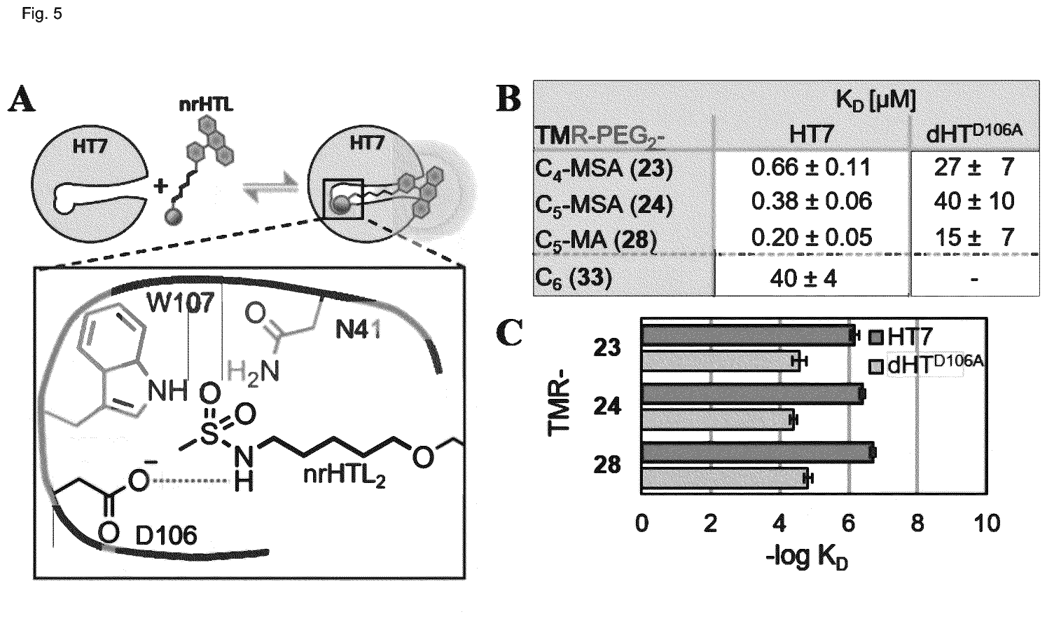

The virtual docking screen aimed for polar interactions between potential nrHTL and the catalytic amino acid D106 of HT7 (FIG. 5A). To prove such binding mode, the affinity of the different nrHTL discovered (TMR-23, -24, and -28) to a HaloTag dead-mutant, where Asp106 was replaced by an alanine residue (dHT^{D106A}), was measured. It was found, that all tested nrHTL offer significantly less affinity to dHT^{D106A} in comparison to the regular HT7 (FIG. 5B). In detail, the difference obtained are about two orders of magnitude (FIG. 5C).

The higher K_{D}-values determined for the nrHTL₁₋₃ binding to dHT^{D106A} is consistent to what was reported in a previous chapter: some initially designed nrHTL ligands such as the C₆ with no moiety (TMR-33) lacked the ability to form hydrogen bonds within the HT7 active site. The measured affinity of TMR-33 to HT7 was 40±4 μM, which is similar to the affinity of nrHTL₁₋₃ (e.g. TMR-24) with dHT^{D106A} (FIG. 5B), proving the necessity of the hydrogen bond with D106 for high-affinity binding to HaloTag proteins. However, a moderate mM affinity is offered by rhodamine-dyes modified with any HTL-like linkers in general, indicating an intrinsic rhodamine affinity of HaloTag proteins.

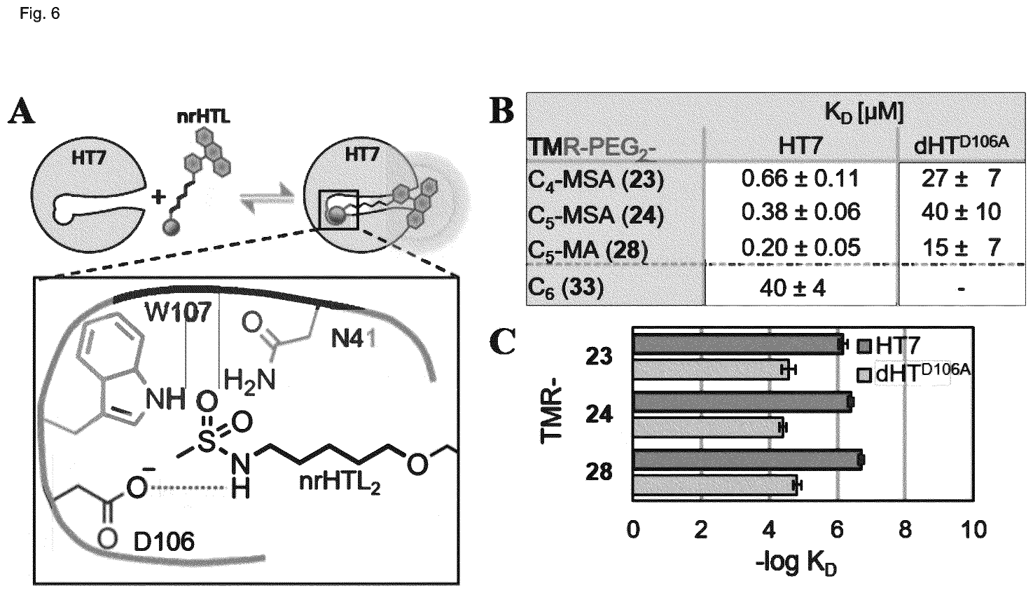

As demonstrated, the discovered binding can be primarily explained by polar interactions between the D106 residue of HT7 and the secondary amine or sulfonamide moieties of the nrHTL. Presumably, the binding events will depend on the protonation state of the nitrogen atoms: it can serve as a hydrogen donor or expose either a positive or negative charge, resulting in potentially attracting/rejecting the negative charge of the D106 residue. Conclusively, binding events might change with the molecule protonation state depending on the pKₐ of the involved chemical functionalities. While the pKₐ of amino acids within a protein binding pocket depends on its microenvironment, the pKₐ of isolated small molecules in water can be estimated with the Schrödinger software tool Epic. FIG. 6A visualizes the possible protonation or deprotonation processes of methylamine (MA) and methylsulfonamide (MSA) moieties in a biological relevant pH range. Calculated pKₐ-values of different aliphatic secondary amine (pKₐ₁) and (sulfon)amide (pKₐ₂) moieties from the nrHTL leads to different overall alkane charge at physiological pH that can be adjusted by introducing electron-withdrawing groups such as methyl fluorides (FIG. 6B).

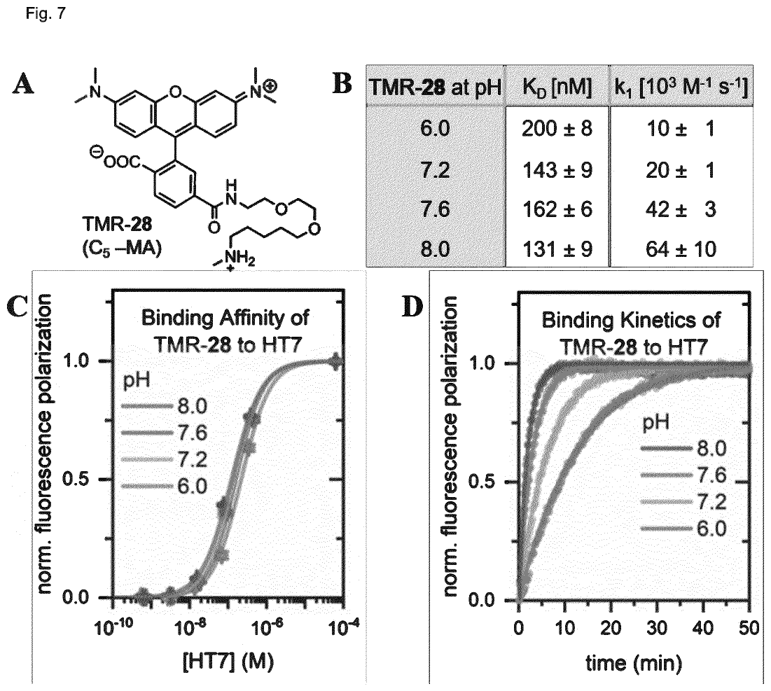

These pKₐ differences are inherent to the nature of both MA and MSA moieties: while sulfonamides as well as acetamides and carbamates are neutral at physiological pH, secondary amines are mostly protonated and therefore positively charged. This might impact the binding mechanism and the binding kinetics of nrHTL. Indeed, TMR-24 presents fast kinetics and is expected to be mostly neutral at physiological pH, whilest TMR-28 is slow and expected positively charged. Its deprotonation was estimated around pH 10.3±0.7 and it was therefore hypothesized, that a pH increase could promote its deprotonation, render it neutral and concomitantly accelerate its HT7 binding. Hence, the binding kinetics and affinity of TMR-28 were investigated at various pH values (FIG. 7).

The binding speed of TMR-28 increases significantly between pH 6.0 and 8.0 by a factor of about 7. At the same time, the binding affinity stays comparable over the same pH-range, considering the limits of the measurement inaccuracy. TMR-23 pH-dependent affinities were recorded equally and similar results were obtained: between pH 7.2 and 8.0 constant K_{D}-values were recorded, only at pH 6.0 a decreased affinity was measured. Thereby, a slight instability of HT7 was detected at mild acidic pH. Despite observing a trend suggesting that an uncharged molecule would bind quicker to HT7, it was not possible to work at a pH allowing TMR-28 to be fully neutral and potentially reach kinetics in the range of TMR-23. Furthermore, the current setup does not allow to distinguish between pH effects on either the protein alone or on the nrHTL. Therefore, it is aimed to cross compare HT7 labeling kinetics with common HT substrates at different pH-values to the nrHTL binding kinetics in order to better interpret the mechanism underlying the fast binding of TMR-23/24 compared to TMR-28. Further experimental assessment of this kinetic interrogation will be provided in chapter 3.3.2 by introducing a triflamide ligand (TMR-25) that is presumably partly negatively charged at a pH around 7 (according to FIG. 6B).

Fluorogenicity of the nrHTL Candidates

High background signal of unbound fluorophores is a central issue in recent PAINT microscopy approaches. Consequently, prolonged acquisition times and the need of optical sectioning techniques are still restricting PAINT as an universal imaging tool. Lately, fluorogenic DNA-PAINT enabled fast 3D-imaging with a 83-fold fluorescence increase upon hybridization. On the protein site, chromophores exhibiting a fluorescence emission increase upon binding to FAPs, were employed in PAINT-microscopy allowing high-quality and long-term PAINT-microscopy in living cells. The capacity of rhodamine-based dyes (Rho) to switch between a non-fluorescent spirocyclic and a fluorescent zwitterionic form upon binding on HT7 makes of the Rho-nrHTL particularly appealing candidates for PAINT-microscopy applications. In this chapter, the propensity of SIR-23 (MSA) and SiR-28 (MA) to switch from spirolactone to zwitterion upon HT7 binding was therefore evaluated.

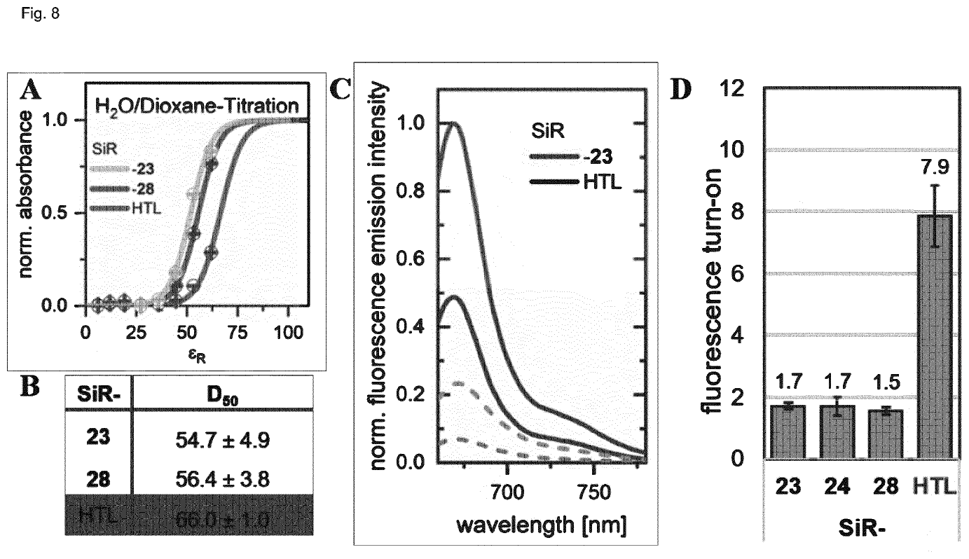

The spirolactone equilibrium can be characterized by measuring the absorbance of the Rho-dyes in different ratio mixture of water/dioxane from which the dielectric constant ε_{R} is known. The half-absorbance value is known to correspond to a dielectric constant D₅₀, that allows to compare different rhodamine-based compounds for their open/closed ratio. It was reported, that fluorescent dyes with a D₅₀-value around 50 are potential candidates for fluorogenic probes whereat SiR highlights a D₅₀ of 59. Water-dioxane titration with nrHTL SiR-23 and SiR-28 (FIG. 8A) delivered D₅₀-values (FIG. 8B), indicating the fluorogenic potential of these SiR-probes. In comparison, the common SiR-HTL substrate presents an equilibrium lying more toward the spirolactone form (FIG. 8A) indicating a potentially higher fluorogenicity than offered by the nrHTL equivalents.

To evaluate the capacity of HT7 to promote the fluorophore to switch to its zwitterionic fluorescent form, the fluorescence emission spectra of SiR-nrHTLs in presence and absence of the HT7 protein were recorded and compared to the common SiR-HTL substrate. From these fluorescence emission spectra (FIG. 8C) the fluorescence turn-on upon HT7 binding observed at the maximal emission wavelength (FIG. 8D) were extracted. As suggested by the D₅₀-values, SiR-23 shows a higher (background) fluorescence emission in absence of HT7 compared to the SiR-HTL substrate. This indicates that the chemical modification of the linker increases the propensity of SiR to be present in a zwitterionic form. Upon binding to HT7, the fluorescence emission of the SiR-nrHTL increases by a factor of 1.5±0.1 to 1.7±0.1, respectively. In comparison, the covalent labeling of SiR using the respective HTL substrate increases the fluorescence emission by a factor of 7.9±2 under similar experimental conditions.

Cellular Staining Using nrHTLs

The next chapter demonstrates the applicability of nrHTLs as fluorescent probes for confocal microscopy. The nrHTL cell membrane permeability was assessed by comparing the staining of fixed and live cells expressing HT7 in their nucleus (NLS-tag). Further, the quantification of signal-to-noise ratios (S/N) was used to compare the labeling specificity between the different neo-developed probes under no-wash conditions. Finally, pulse-chase experiments were employed to verify the kinetics of transient (un-)staining of HT7 by nrHTLs, further explored via fluorescence recovery after photobleaching (FRAP) experiments.

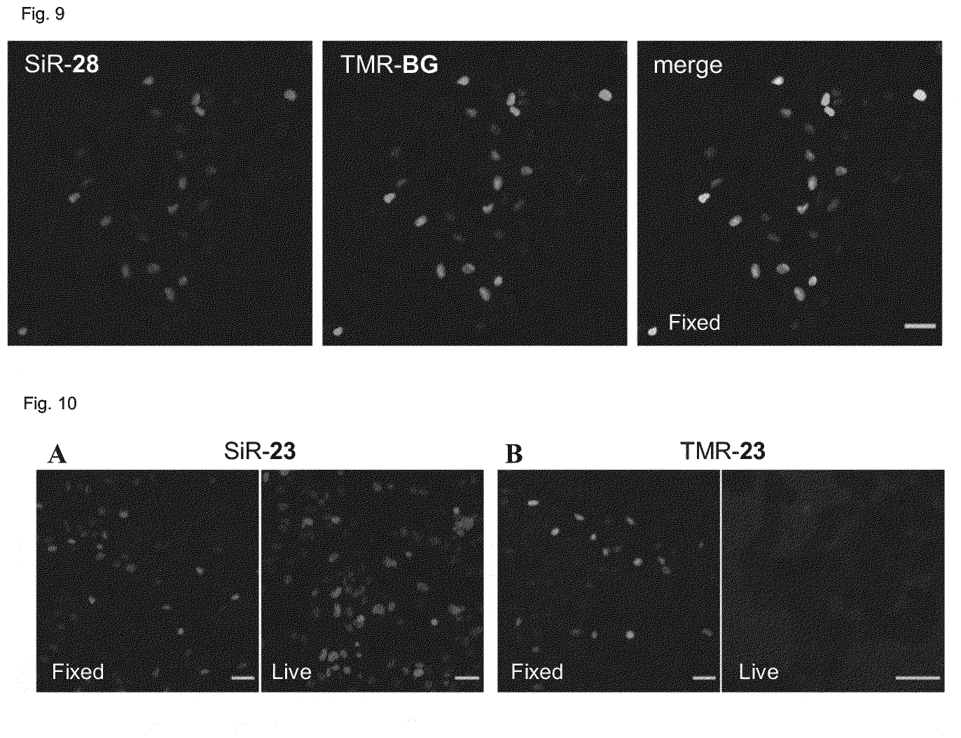

In fixed U-2 OS cells expressing HaloTag7-SNAP-NLS, all nrHTL-modified TMR, CPy and SiR probes delivered a clear fluorescent signal localized at the nuclei and quantitatively comparable to covalent labeling using their respective HTL fluorophore substrates. Only Atto565-23 shows unspecific staining within the whole cell. However, a similar result was obtained from Atto565-HTL substrate, indicating that the dye itself tends to bind unpacifically to some intracellular molecules. Further, a TMR-benzyl guanine (TMR-BG) counterstaining of the fused SNAP protein allowed to demonstrate the proper co-localization of the nrHTL, exemplified by SiR-28 (C₅-MA, FIG. 9).

The staining comparison between fixed and live U-2 OS cells (HaloTag7-SNAP-NLS) with SiR-23 (FIG. 10A) shows specific and similar nuclear staining under both conditions, with minor intracellular background. In contrast, TMR-23 is not able to stain live cells with intact membranes while chemically arrested cells nuclei are properly stained (FIG. 10B), revealing the difficulty of TMR-23 to pass membranes. This finding also applies to all TMR-nrHTL including -24 and -28, as well as Atto565-23.

### EXAMPLE 3

Fluorescence Increase through Dye Exchange and Engineered HT Variants

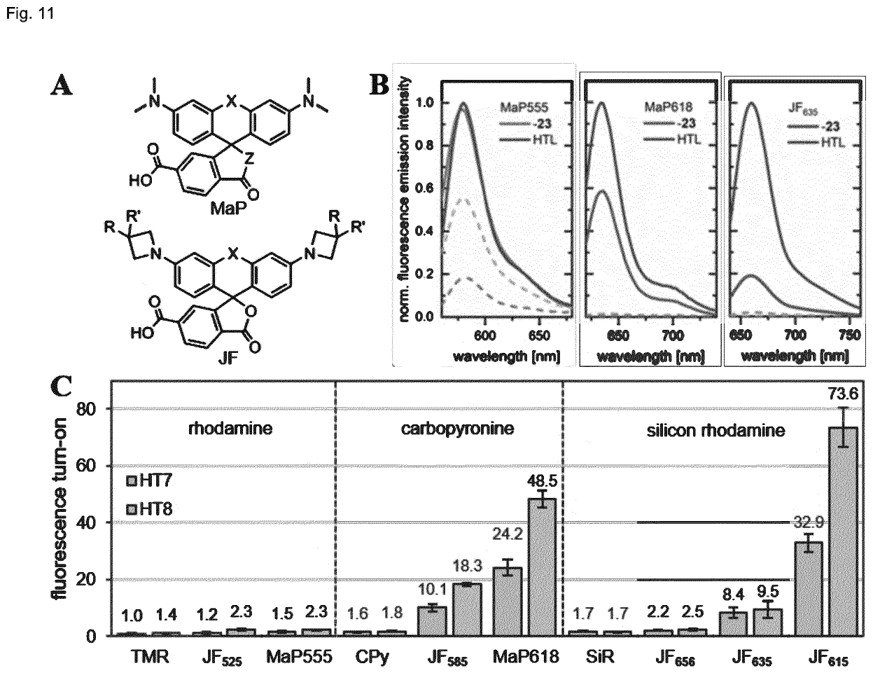

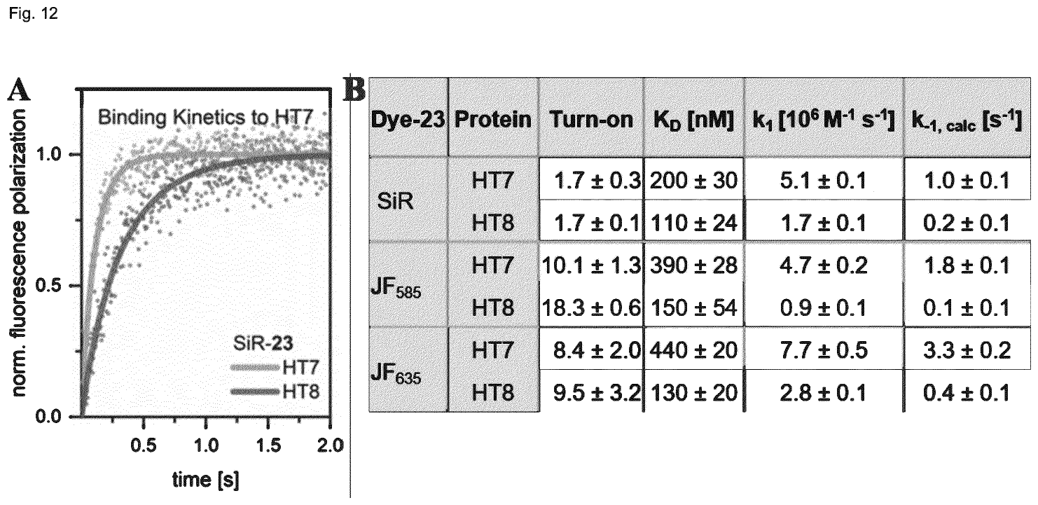

With the scope of finding the most valuable probe for live-cell PAINT imaging, nrHTL₁ 23 was coupled to a large panel of both MaP and JF dyes (FIG. 11A). The results described in chapter 3.2.4 suggest, that the modification of the terminal moiety of the common HTL substrate shifts the open/closed equilibrium of the rhodamine dyes toward the zwitterionic form, reducing SiR fluorogenicity upon HT7 binding. Therefore, the hypothesis was made, that fluorophores with a higher propensity to form spirolactons could potentially provide nrHTL with fluorogenicity in the range of SiR-HTL and enhance the cell permeability. Additionally, the recent in-house engineering of HaloTag led to the discovery of a promising

HaloTag variant (HaloTag8, HT8) characterized for its ability to significantly increase the brightness of rhodamine-based fluorophores upon binding.

Among all tested Rho-nrHTL₁ (C₄-MSA) combinations, the highest turn-on was obtained for orange to far-red shifted dyes that are known to predominantly adapt to the non-fluorescent spirolactone form such as MaP618 and JF₆₁₅ (FIG. 11C). For instance, an intermediate turn-on upon HaloTag7 binding of 29.1±2.3 was measured for MaP618-23. Preferentially, the nrHTL fluorophores were used in combination with HT8 which brought an additional turn-on of roughly two times, as compared to HT7. However, MaP618-HTL is reported to highlight a 1,000× fluorescence turn-on with HT7 in-vitro and side-by-side comparison exhibited that MaP618-nrHTL₁ grasps only 60% of the signal intensity that is reached by covalently labeling of HT7 (FIG. 11B, middle panel).

On the other hand, the nrHTL modification on a rhodamine scaffold (e.g. MaP555 or JF₅₂₅) barely shows any turn-on. The reason here is, that these green to yellow fluorescent nrHTL already exhibit high fluorescence intensity prior to HaloTag binding (FIG. 11B, left panel) corroborating the initial observation made about the overall capacity of the nrHTL moiety to switch the equilibrium of rhodamine-based fluorophore towards their zwitterionic form. In comparison, the turn-on for equivalent HTL substrates was reported of 35× for MaP555. Applying the JF-strategy onto the SiR scaffold switches the open/close equilibrium so much towards the spirolacton form that such a fluorophore-HTL highlights an extreme fluorogenicity upon binding to HT proteins. Despite the fact, that JF₆₁₅-23 delivers the highest turn-on (FIG. 11C), it shows less than 4% fluorescence intensity after HT7 binding compared to JF₆₁₅-HTL covalently labeled to HaloTag7. Finally, JF₆₃₅-23, shows a significant turn-on of 8.4±2.0 (FIG. 11C) and a decent brightness in presence of HT7 (FIG. 11B, right panel), representing the best compromise in between fluorogenicity and overall signal brightness among the SiR-based fluorophores.

The binding affinities of all fluorogenic variants of the nrHTL₁ 23 were measured as previously explained leading to K_{D} ranging from 120±8 nM (JF₆₅₆) to 910±260 nM (MaP555). This stands in a good agreement with the binding affinities reported above for such nrHTLs to HT7, following the trend that rhodamine-derived dyes show weaker binding, than carbopyronine or silicon rhodamine probes.

Selected as most promising fluorogenic nrHTLs for PAINT microscopy, the binding kinetics of JF₅₈₅-23 and JF₆₃₅-23 were determined (FIG. 12). In case of JF₅₈₅-23 the favourable fluorescence turn-on seems to engender mildly reduced binding kinetics. However, JF₆₃₅-23 highlights that a binding speed comparable with SiR-23 can be obtained with fluorogenic nrHTLs (FIG. 12B). That makes it exceptionally beneficial as a potential PAINT probe.

Overall, the nrHTL fluorophore bound with higher affinity to HT8 than to HT7, however a significantly decreased binding speed to HT8 was detected (FIG. 12A). In combination this yields in a poor off-rate below the aimed 1 s⁻¹ range.

Introducing Novel Terminal Moieties and Identifying their Binding Behavior

Based on an initial virtual docking screen, novel non-reactive HaloTag ligands were discovered. During in-cellulo imaging nrHTLs containing methylsulfonamide moieties emerged as powerful probes that offer a high binding affinity, quick binding and precise cell staining, the latter is especially evident in combination with fluorogenic fluorophores. These properties make them appealing for super-resolution imaging applications such as STED and PAINT for which demonstrations were brought during this work. Concretizing the potential such nrHTLs are carrying, the initial design of the terminal nrHTL moiety was revised, aiming for potential better ligands. Moreover, diversifying the toolbox of available ligands is supporting the inventors' mechanistic understanding of the HaloTag-nrHTL interaction since, for instance, the pH-dependence of the binding parameters described in chapter 3.2.3 was not finally conclusive yet.

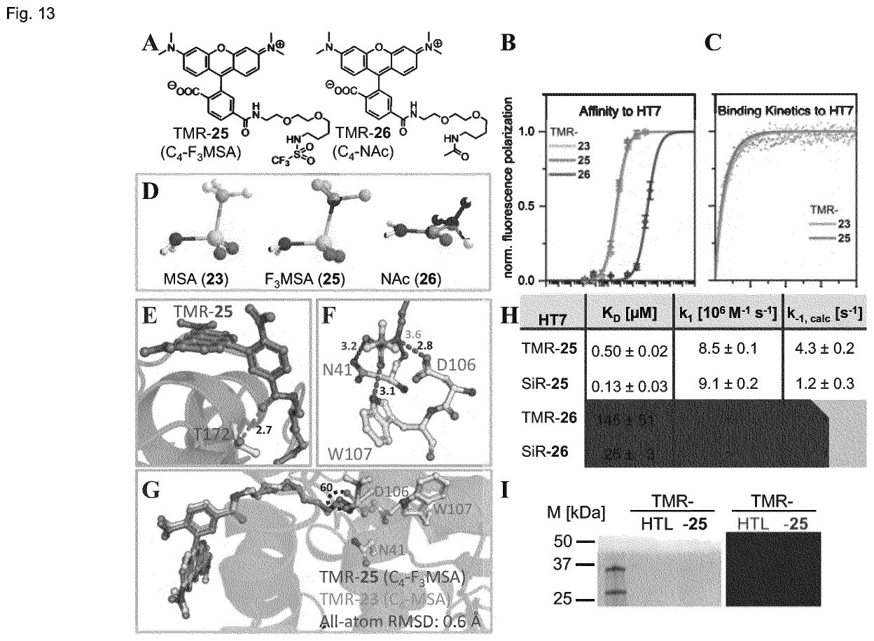

Accordingly, two more (sulfon-)amide ligands (25, H₂N-PEG₂-C₄-NH—SO₂—CF₃ and 26 H₂N-PEG₂-C₄-NAc, FIG. 13A) were synthesized and coupled to TMR and SiR. The novel ligands were fully biochemically characterized as previously explained for 23, 24 and 28 (FIG. 13). Notably, the triflamide 25 shows a high degree of structural similarity to 23 with the difference of the highly electron-withdrawing trifluoromethyl group attached to the sulfonamide. Reducing the electron density also decreases the pKₐ₂ of the amide as previously displayed (FIG. 6) leaving the ligand partially deprotonated (i.e. negatively charged). On the other hand, 26 represents an uncharged acetamide, offering hydrogen-donor potential though the geometry (planar) and orbital hybridisation (sp²) of amides is in stark contrast compared to amines and sulfonamides (tetrahedral, sp³, sees FIG. 13D).

No binding was observed for TMR-/SiR-26, hinting an incompatibility of amides to either enter or bind to the HT active centre in the same way as demonstrated for methylamines and sulfonamides. In contrast, TMR- and SiR-25 present binding properties (affinity, kinetics) analogous to their corresponding nrHTL 23 (FIG. 13B, C, H). Noticeably, the predicted partial negative charge of 25 does not affect the binding kinetics. The crystal structure of TMR-25 in complex with HT7 highlights similar protein/ligand interactions compared to TMR-23: the binding moiety shows polar contacts with the catalytic site residues N41, D106 and W107 as well as a putative hydrogen bond to T172, placing the xanthene core on the HT7 surface (FIG. 13E, F). However, a 60° rotation of the triflamide group respective to the MSA moiety of TMR-23 was discovered to accommodate the space-demanding trifluoromethane group into the binding site. This results in placing the whole ligand inside HT7 in a slightly shifted conformation respective to TMR-23, illustrated by an all-atom root-mean-square deviation (RMSD) of 0.6 Å (FIG. 13G). Finally, the reversible nature of the binding of -25 to HT7 was demonstrated by SDS-PAGE and in-gel fluorescence imaging (FIG. 131), concluding that fluorophore-25 derivates (further termed as nrHTLA) harbor a great potential as PAINT probes.

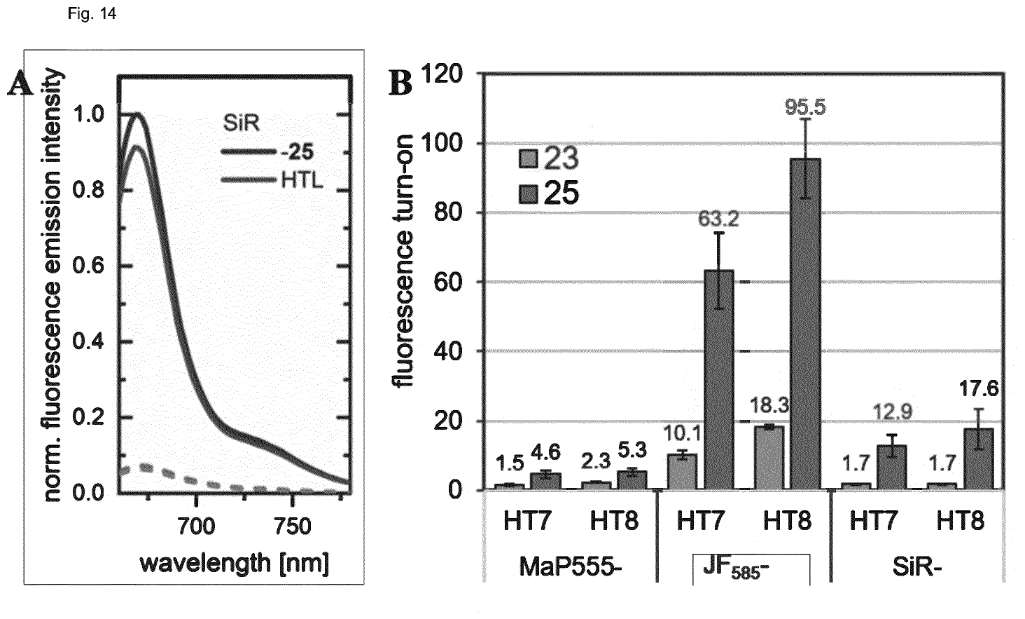

Further, MaP555 and JF₅₈₅ were coupled to nrHTL 25 to investigate the impact of the triflamide moiety on their fluorogenicity upon HT7 or HT8 binding (FIG. 14). The fluorogenic effect was improved for both dyes by a factor of 2.5 and 5, respectively. Unexpectedly, the fluorogenicity characterizing the SiR-HTL substrate with HT7 was recovered for SIR-25 in contrast to SiR-23 (FIG. 14A). SiR-25 is relatively non-fluorescent in a buffered solution but exhibit a more then 10-fold turn-on upon HaloTag7 binding. The highest fluorescence turn-on was measured with JF₅₈₅-25 in combination with HT8 highlighting a 95.5±11.3 fold increase (FIG. 14B).

In summary, the fluorescent triflamide probes (25) showed similar binding characteristics to HT7 than methansulfonamide probes (-23, -24), but improved the nrHTL-HaloTag system thanks to a better in-vitro turn-on. A fluorescence increases of up to 92× when used in combination with HT8 was recorded. However, in-cellulo, nuclear staining presents a reduced signal-to-noise ratio because of cytosolic background signal when the probe was used at 500 nM concentration for staining over-night. Nevertheless, staining of H₂B-HT8 with MaP555-, JF₅₈₅- and SiR-25 leads to superior brightness, highlighting the suitability of such probe for live-cell confocal microscopy. Herein, the application in concentrations below 500 nM is highly suggested to make perfect use of the fluorogenic potential of those molecules and reduce unwanted background signal.

### Circular-Permuted Halo Tag as an Alternative Binding Partner

Nowadays, resolving distances down to approximately single nanometres is routinely achievable from a technical point of view, as demonstrated recently by DNA-PAINT or MINFLUX (Gwosch, K. C. et al., Nat. Methods 2020, 17, P. 217) fluorescence nanoscopy. Such distances represent the size of proteins, such as the HaloTag, and question the capacity to place a fluorophore close enough to the object of interest via genetic tagging, where the N and C-termini of proteins being rarely close to the fluorescent moiety. Circular permutation (cp) is a protein engineering approach allowing to change the order of the amino acid sequence of a certain protein by linking its initial N and C-termini via a short linker and opening new termini where wished and tolerated. The cp-variant theoretically own similar 3D-structure and functionality. Recently, cpHaloTag7 at position 143 was used in the development of chemigenetic voltage-indicators. Hereby, the cpHalo strategy aimed opening new termini close by the fluorophore binding site. Similar works were undergoing in-house, offering different cp-options to bring the HT7-bound fluorophore in close proximity to a sub-cellular target aiming for a potential resolution increase in SRM.

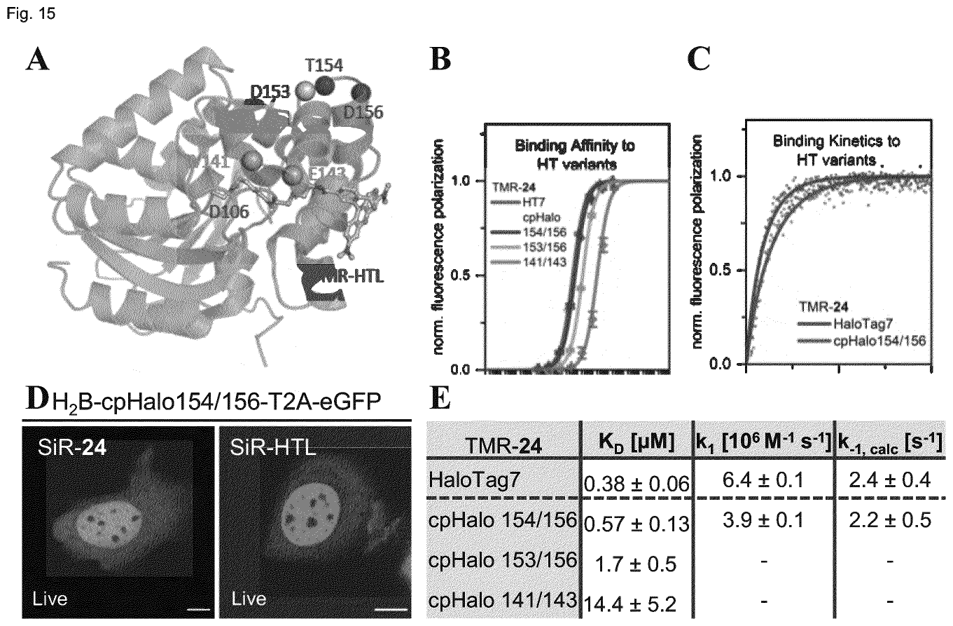

In the following, the binding of nrHTL probes to three different cpHaloTag proteins (141/143, 153/156 and 154/156, FIG. 15A) is characterized in order to verify if these cp-variants retain similar binding characteristics to nrHTLs as HaloTag7. However, investigation of the binding affinity with TMR-24 delivered similar results only for cpHT7-154/156 compared to regular HT7 (FIG. 15B). The following experiments therefore focused on this particular protein: measuring binding kinetics (FIG. 15C) in combination with TMR-24 delivered again comparable results. Moreover, staining of H₂B-cpHaloTag154/156-T2A-eGFP expressed in living U-2 OS cells was demonstrated. Therein, nrHTL SiR-24 exhibits analogous results to covalent labeling using SiR-nrHTL (FIG. 15D). Conclusively, despite a slight reduction of the dissociation constant and the on-rate (FIG. 15E), SiR-24 led to specific nuclear staining making cpHaloTag154/156 an attractive target for nrHTL probes in future SRM applications.

### EXAMPLE 4

### Development of an Orthogonal Halo Tag Protein/Ligand Systems

Orthogonal fluorescent staining is of great interest for multi-colour imaging in a biological context. The visualization of several targets at the same time does not only increase the amount of information gathered from fluorescence microscopy, but also enables the temporal study of interactions. Herein, SLPs allow to tag synthetic fluorophores to proteins of interest that offer narrow emission spectra allowing multiplexing in high resolution imaging setups. Up to now, an orthogonal variant of SNAP-tag, dubbed as CLIP-tag, was engineered to allow orthogonal fluorescent labeling. Nevertheless, HaloTag remains the SLP mostly used, notably in animal models, and an orthogonal version of HaloTag would therefore be highly appreciated.

Considering the progress made with the nrHTL approach and the mechanistic knowledge gained in ligand design, the possibility opened up to engineer an orthogonal HaloTag technology using transiently binding ligands selectively targeting two different HaloTag protein variant with sufficient specificity to allow orthogonal co-staining. Thanks to the reversible binding an orthogonal ligand with the required kinetic properties would be highly desirable for dual-colour HT-PAINT microscopy.

Screening for Potential dHT7 Ligands

As demonstrated earlier in this work, the single amino acid exchange of the D106 residue of HT7 causes a drop in binding affinity for all neo-discovered nrHTL. Therefore, ten dead variants of HaloTag were generated by site-directed mutagenesis, targeting the catalytic D106 residue mutated into G, A, V, I, L, C, S, T N or E residues (dHT7^{D106X}), with the goal to find a ligand that bounds with moderate affinity to such mutant but not to the native HT7. The proteins were produced, purified and quality checked prior to characterization.

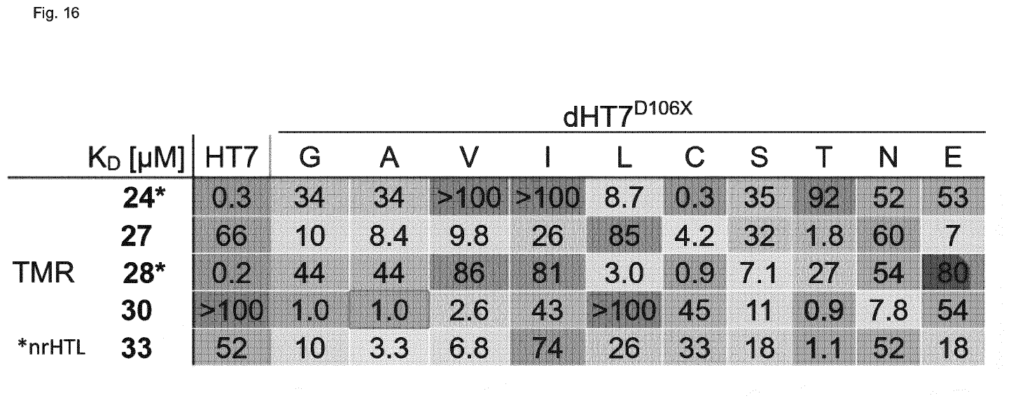

The binding affinity of initially designed TMR-nrHTL candidates (TMR-24, -27, -28, -30 and -33) led to measured K_{D} ranging between 0.2 and >100 μM (FIG. 16). Herein, the variant HT7^{D106C} revealed to be an equivalent binder to nrHTL TMR-24 and -28 than the native HT7. Interestingly, the moderate binding affinity of TMR-30 (C₄—OH) to some dHT7^{D106X} variants (e.g. D106G, A and T mutants), while being a poor ligand for the native HT7, makes this ligand a good starting point to develop a dHT7^{D106X} ligand (termed dHTL) that is orthogonal to HT7. The following chapter focusses on the rational improvement of TMR-30 (C₄—OH) to specifically bind dHT7^{D106A} with significantly higher affinity (further referenced as dHT7) over native HT7. In contrast to the nrHTL system, the transient interaction of the fluorescent probes is ensured on the protein level by the HaloTag dead-mutant.

### Improvement and Characterization of Ligands for dHT7 Specific Binding

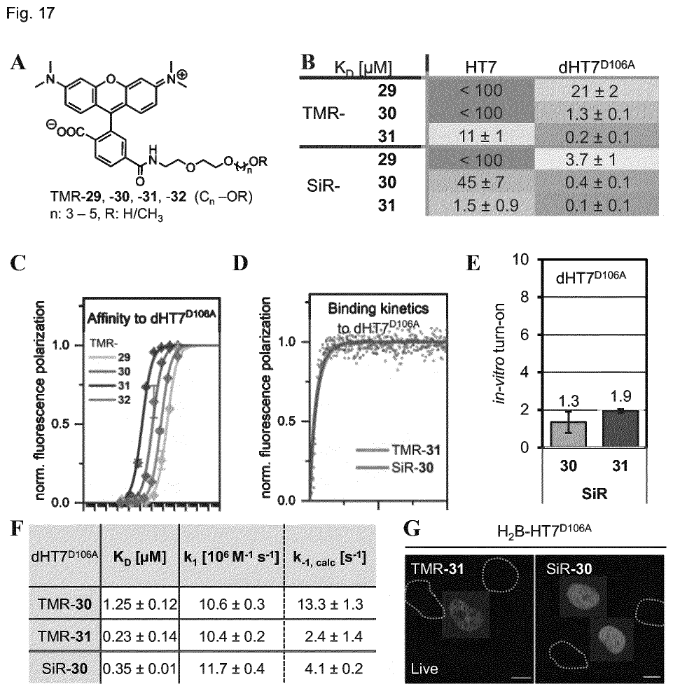

In order to optimize a ligand for dHT7, it was hypothesized that the linker length of potential dHTL 30 might not be optimal in the protein active site, in which the D106 was replaced by a residue of a smaller size. To proof such hypothesis, dHTL candidates 29 and 31, presenting ₃ and Cs linker length in front of the hydroxy binding moiety (FIG. 17A), were synthesized and coupled to TMR and SiR fluorophores. To understand the ligands binding mode, additionally a methoxy-bearing ligand (32, C₄—OCH₃) was produced to mask the hydrogen donor potential of nrHTL 30.

The following work focused on comparing the neo-synthesized potential dHTL affinity to dHT7^{D106A} and HT7, with the scope of reaching a high orthogonality (FIG. 17B). The binding affinity of TMR-Cn-OH ligands to dHT7^{D106A} increases with the linker length, almost a log-scale per additional methylene group (FIG. 17C). As previously described, using SiR over TMR increases the binding. However, a similar trend was also observed for HT7, albeit to a lesser extent. The replacement of the terminal hydroxy group by a methoxy moiety decreases the binding affinity to dHT7^{D106A}, suggesting once more that a polar interaction might drive the binding process (FIG. 17B). Overall, SiR-30 (dHTL₁) and TMR/SIR-31 (dHTL₂) exhibit sub-micromolar affinity for dHT7^{D106A} but SiR-31 showed also a decent binding affinity to native HT7. That indicates that SiR-31 most likely cannot be considered as an orthogonal ligand even thought it might be a potent dHTL (and a nrHTL simultaneously).

The binding kinetics and fluorescence turn-on of the novel dHTL were assessed for their dHT7^{D106A} target (FIG. 17C and D, respectively). Herein, the binding of TMR-31 and SiR-30 occur even faster than the corresponding nrHTL binding kinetics to HT7 reported previously (FIG. 17D and F). The superior on- and off-rates detected potentially open the doors for dual-colour staining that might be ideal for PAINT-imaging. However, no significant turn-on was reported for these novel SiR-dHTL (FIG. 17E) just as for SiR-nrHTL previously. In conclusion, it was possible to design a novel class of non-covalent HTL offering high affinity and binding speed to dHT7^{D106A} in-vitro and laying the foundation for an orthogonal nrHTL/dHTL system, whereat further refinement of the fluorescence turn-on will be necessary.

As an initial proof-of-concept, H₂B-HT7^{D106A}-T2A-meGFP expressing live cells were stained using dHTL₁ SiR-30 and dHTL₂ TMR-30 and imaged under no-wash conditions (FIG. 17G). Both probes reveal sufficient cell permeability and a specific nuclear staining with almost no background signal. Final evaluation of the mutually orthogonal nrHTL/dHTL system is brought in the following chapter by in-cellulo staining of cells expressing both proteins at different intracellular compartments.

### Material and Methods

### General Synthesis

Chemical reagents for synthesis were purchased from commercial suppliers (Acros, Fluka, Merk, Roth, Sigma-Aldrich, TCI, TOCRIS) and used without further purification. Reactions performed under air and moisture exclusion were carried out in heat-dried glassware and under inert argon or N₂ atmosphere using Schlenk techniques. Water-free solvents acetonitrile (MeCN), dichloromethane (DCM), dimethylformamide (DMF), ethanol (EtOH), methanol (MeOH) and tetrahydrofuran (THF) were stored over molecular sieves and used directly from a sealed-bottle. Dimethylsulfoxide-de was taken freshly from 0.75 mL glass-ampoules (Roth), stored in a closed vial and used up within two days. 6-Carboxy modified rhodamine dyes were obtained custom synthesized from Atto-Tec and Spirochrome AG or were kindly provided by Bettina Mathes and Dominik Schmidt.

Evaporation in vacuo was achieved at 40° C. and 10-850 mbar at a rotary evaporator (Buchi). The compounds purified by high-performance liquid chromatography (HPLC) were flash frozen and lyophilized on a lyophilizer (Christ) equipped with a vacuum pump (Vacuubrand).

### Preparative Chromatographic Methods

Flash column purification was performed using a Biotage (Isolera™ One) flash system equipped with pre-packed SiO₂ columns (SiliaSep™ Flash Cartridges, 40-63 μm, 60 Å). Depending on the batch size 12 g, 25 g or 40 g columns with 40, 75 or 100 mL min⁻¹ flow rate were used, respectively. Typical gradients were 10 to 50% ethyl acetate (EtOAc) in n-hexane (hex) or 1 to 10% MeOH in DCM within 10 column volumes (CV).

Small-scale preparative reversed-phase high-performance liquid chromatography (RP-HPLC) was carried on an UltiMate 3000 system (Thermo Fisher Scientific). Column: C₁₈ 5 μm, 21.2×250 mm (Supelco). Buffer A: 0.1% TFA in MiliQ® water (ddH₂O), buffer B: MeCN. Typical gradient was from 20% to 90% B within 45 min with 8 mL min⁻¹ flow. It was equipped with a 2998 PDA detector for automated product collection based on the absorption wavelength of fluorescent labels (at 280, 550, 620 or 650 nm, respectively).

Large-scale RP-HPLC-MS purification (>3 mg) was carried-out with a LCMS-2020 unit (Shimadzu) coupled with a prominence LC-20AP UFLC (Shimadzu). Column: C₁₈ 5 μm, 30×250 mm (Shimadzu). Buffer A: 0.1% FA in ddH₂O, buffer B: MeCN. Typical gradient was from 10% to 90% B within 45 min with 20 mL min⁻¹ flow. Product visualization was reached on an SPD-M20A UV-VIS photodiode array detector and the desired compounds were collected based on the calc. mass-to-charge ratio (m/z) using a DUIS-2020 dual ion source (Shimadzu).

### Analytical Chromatographic Methods

Reaction progress and chromatography fractions were monitored by analytical thin-layer chromatography (TLC) or liquid chromatography coupled to mass spectrometry (LC-MS). TLC was accomplished on commercially available SiO₂-plates (POLYGRAM® SIL G/UV254, 0.2 mm layer pre-coated polyester sheet, 40×80 mm) and visualized by using 254 nm UV-light or TLC staining solutions (see Table 1) and gentle heating.

TABLE 1
Recipe of staining solutions for thin-layer chromatography

| TLC stain | Recipe |
|---|---|
| KMnO₄ | 1.5 g KMnO₄, 10 g K₂CO₃, 1.25 mL 10% NaOH |
| | in 200 mL ddH₂O |
| Ninhydrin | 5 g Ninhydrin in 100 mL EtOH w. 3% (v/v) AcOH |
| Vanillin | 15 g Vanillin in 250 mL EtOH w. 1% (v/v) conc. H₂SO₄ |

LC-MS was performed on a LCMS2020 (Shimadzu) connected to a Nexera UHPLC system. Column: C₁₈ 1.7 μm, 50×2.1 mm (ACQUITY UPLC BEH, Waters). Buffer A: 0.1% FA/ddH₂O, buffer B: MeCN. Typical gradient was from 10% to 90% B within 6 min with 0.5 mL min⁻¹ flow.

Analytical RP-HPLC was used to evaluate fluorescent compound purity. Samples were prepared in 5 μM concentration in 5% H₂O in MeCN with 0.1% (v/v) TFA. It was carried out on Waters e2695 system equipped with a 2998 PDA detector. Column: C₁₈ 4 μm, 3.9×150 mm, 60 Å (Nova-Pak). Buffer A: 0.1% TFA in ddH₂O, buffer B: MeCN. Gradient: Hold 1.5 min at 20% B and increase within 12.5 min to 80% B, 1.23 mL min⁻¹ flow. The peaks at the fluorophore-specific absorption wavelength were manually integrated. Compounds with a main peak intensity of ≥ 95% were considered as suitable for the following experiments.

Biochemical Methods and in-vitro Characterization

### UV-Vis Spectroscopy

The concentration of fluorophore-ligands (HTL and nrHTLs) was determined by measuring the UV-Vis absorption of the dye at their maximum absorption wavelength and using Lambert-Beer's law:

Abs=c·d·ε (1)

Abs: absorption at λₘₐₓ [AU], c: concentration [mol/l], d: pathway length [cm], extinction coefficient [l/mol·cm]

Absorbance were measured with a Nanodrop 2000cTM spectrophotometer using a drop of solution (d=0.1 cm) or a polystyrene cuvette (Sarstedt, 10×4×45 mm). Samples were prepared in PBS pH 7.4 (Gibco), 0.1% SDS in activity buffer (composition see Table 3) or 0.1% TFA in EtOH, depending of the fluorophore properties and according to literature precedents. The previously in-house characterized extinction coefficients & were used to calculate the concentration of neo-synthesized fluorophore ligands (for spectral properties of all used fluorophores see Table 2). Fluorescent molecules for following experiments were prepared as stock solutions in dry DMSO (>1 mM) and diluted such that the DMSO concentration did not exceed 1% (v/v).

TABLE 2
Spectral properties of fluorophores used for UV/Vis spectroscopy

| Fluorophore | λ_{abs} [nm] | ε [M⁻¹ · cm⁻¹] | in solvent |
|---|---|---|---|
| JF₅₂₅ | 526 | 138,000 | 0.1% SDS |
| TMR | 555 | 89,000 | PBS |
| MaP555 | 555 | 142,000 | 0.1% SDS |
| Atto565 | 564 | 120,000 | PBS |
| JF₅₈₅ | 586 | 52,500 | 0.1% SDS |
| CPy | 609 | 152,000 | 0.1% SDS |
| MaP618 | 613 | 5,500 | 0.1% SDS |
| JF₆₁₅ | 615 | 7,000 | 0.1% TFA in EtOH |
| JF₆₃₅ | 638 | 17,000 | 0.1% SDS |
| SiR | 645 | 140,000 | 0.1% SDS |
| JF₆₅₆ | 649 | 106,000 | 0.1% SDS |

### Plasmid Generation

Plasmids were obtained by molecular cloning using the Gibson Assembly (GA) (Gibson, D. G. et al, Nat. Methods 2009, 6, P. 343) method or a site-directed mutagenesis kit (NEB). All DNA-primers were designed using the Geneious software (Biomatters) or online-resources from NEB and further custom-synthesized (Merck). For protein production in Escherichia coli a modified pET-51b(+) plasmid (Novagen) was employed in order to fuse a Histidine tag (10×) and a Tobacco Etch Virus (TEV) protease cleavage site in N-terminal of the protein of interest (POI). For mammalian cells protein expression, the template plasmid pcDNA5/FRT/TO (ThermoFisher Scientific) was employed. All template plasmids were kindly provided by Dr. Julien Hiblot or Dr. Michelle Frei.

Site-directed mutagenesis were performed to create HT7^{D106X} variants (X=G, V, I, E or T) of pET-51b(+) His10x-TEV-HT7 and to introduce the HT7^{D106A} mutation into pcDNA5/FRT/TO H₂B-HT7-T2A-eGFP and IgKchL-HA-HT7-myc-PDGFRtmb constructs according to the manufacturer protocol (NEB). In short, DNA amplified by PCR was submitted to parental template DNA Dpnl digestion, phosphorylation and ligation (KDL treatment). ˜200 ng of plasmid DNA was transformed into chemically-competent E. coli cells (Q5® Site-Directed Mutagenesis Kit) by heat-shock (40° C., 45 s) prior to recovery (Super Optimal broth with Catabolite repression, SOC) and selection on Luria-Bertani broth medium containing 100 μg ml⁻¹ ampicillin agar plates (LB^{Amp}) at 37° C. over-night.

Gibson Assembly was employed to replace the eGFP DNA fragments in a pcDNA5/FRT/TO H₂B-HT7^{D106A}-T2A-eGFP plasmids by a N-terminally HT7-tagged LaminB gene. PCRs were performed as previously explained. After amplification verification, remaining template DNA was eliminated by enzymatic digestion (DpnI FastDigest, Thermo Fisher Scientific) and the desired PCR fragments were purified using minElute PCR purification kit (Qiagen). GA reaction was performed according to the published protocol by incubation of 1 h at 50° C. Transformation were performed by electroporation of electrocompetent E.cloni® (Lucigene) using Gene Pulser® cuvettes (Bio-Rad) and an Eporator® (Eppendorf, 2200 V). Cells were grown on LB^{Amp} agar plates at 37° C. over-night.

Single bacterial colonies were picked and grown in 5 mL sterile LB^{Amp} at 37° C. over-night shaking at 220 rpm in a 24-deep-well plate. The desired plasmids were purified using the QIAPrep spin miniprep kit (Qiagen) according to the manufacturers protocol. All sequences were verified by Sanger sequencing (Eurofins Scientific) assisted by the Geneious software.

### Protein Production and Purification

The pET51b(+) His 10x-TEV-POI plasmids were transformed as previously described in electrocompetent E. coli strain BL21(DE3)-pLysS and grown on LB-agar^{Amp} at 37° C. over-night. 5-10 colonies were picked to guarantee an equal expression level and grown in 3 mL sterile LB^{Amp} pre-culture at 37° C. over-night shaking at 220 rpm. On the next day, 1 L LB^{Amp} was inoculated with 1 mL pre-culture and grown at 37 ° C. and 220 rpm until an optical density at 600 nm (OD₆₀₀) of 0.6 was reached. Then, the temperature was reduced to 18° C. and protein production was induced with 0.5 mM isopropyl β-thiogalacopyranoside (IPTG). After overnight expression, the cells were harvested by centrifugation (4000× g, 15 min, 4° C.), resuspended in 30 mL ice-cold extraction buffer (composition see Table 3) including 1 mM phenylmethylsulphonyl fluoride (PMFS) and 0.25 mg/mL lysozyme. Cell lysis was carried out by sonication (SONOPLUS, 7 min, 50% on/off cycles, 70% amplitude) at 4° C. The lysate was cleared from the cell debris by centrifugation (20 min, 50,000× g, 4° C.) and cautiously collected in fresh 50 mL Falcon tubes. The desired protein was purified by immobilized metal affinity chromatography (IMAC) on an AEKTAPure M fast protein liquid chromatography (FPLC) system (GE-healthcare). Therefore, a FF-HisTrap column (GE-healthcare) was equilibrated with His wash buffer (composition see Table 3). After binding the crude lysate to the column and extensive wash (6 CV), the desired protein was eluted using His elution buffer (composition see Table 3) and the desired fraction was collected based on UV/Vis absorbance in a ˜30 mL fraction. Further the buffer was change on a HiTrap® 26/10 Desalting Column (GE-Healthcare) to activity buffer on the same instrument. The protein solution was concentrated with Amicon® Ultra 15 mL Centrifugal Filters MWKO: 10,000 kDa (5-20 min, 4,500 rpm, 4° C.) to ˜500 μM, flash frozen in liquid nitrogen as 100 μL aliquot and stored at −80° C.

### Protein-Labeling, Electrophoresis and Visualization on Protein Gels

The purified HT7 protein (5 μM) was labeled using 4× excess (20 μM) of the fluorescent non-reactive HaloTag Ligands (nrHTL) in comparison to the corresponding common HTL. Labeling reaction was carried out in activity buffer for 30 min at 37° C.

Prior to electrophoresis, the protein samples were prepared in a Laemmli sample buffer (Table 3), including 10 mM dithiothreitol (DTT), and fully denatured at 95° C. for 10 min. 5 μg fluorescently-labeled proteins were loaded onto precast polyacrylamide gels (mini-PROTEAN® TGX™, 4-20%, 10-well, 30 μL/well, Bio-Rad) in a PROTEAN® cell (Bio-Rad) chamber. For protein purity evaluation, ˜50 μg of isolated protein was applied onto stain-free gels (mini-PROTEAN® TGX™ Stain-Free™, 4- 20%, 10-well, 30 μL/well, Bio-Rad). PrecisionPlus Protein™ All Blue (Bio-Rad) pre-stained marker was used as a reference. Electrophoresis was run for ˜35 min at 220 V in 1× TGS running buffer (fisher bioreagents).

Afterwards, in-gel fluorescence was performed at a ChemiDoc XRS+imager (Bio-Rad). Stain-free gels were exposed for 1 min to UV light to reveal unlabeled protein (purity verification) and imaged with λ_{ext/em}=302 nm/590±110 nm. Results of protein in-vitro labeling with fluorophore-ligands were revealed using the following channel parameters; TMR: λ_{ext/em}=520-545/605±50 nm, SiR: λ_{ext/em}=625650/695±55 nm. Finally, these gels were stained in Coomassie Brilliant Blue R-250 staining solution (Bio-Rad) over-night, washed with ddH₂O and imaged by trans-white illumination (λₑₘ=590±110 nm) at the same imager. The stain-free property of the gels can not be used in parallel to fluorescent labeling of the proteins.

### Fluorescence Polarization Assay

The fluorescent nrHTL (10 nM) were titrated using purified HaloTag proteins [0-200 μM] in activity buffer containing 1% (w/v) Bovine Serum Albumin (BSA, Fraktion V, Roth), in a black flat bottom 380-well plate (Greiner, 20 μL) and at 37° C. The fluorescence polarization (FP) was measured on a microplate reader (Spark20-Tecan) by exciting the TMR at 535±12.5 nm and recording the emission at 595±17.5 nm. Gain was determined as optimum at 67%. The SiR fluorophore was excited at 610±10 nm and recording the emission at 690±10 nm (optimal gain: 138%).

The data from three technical replicates per protein concentration were averaged and normalized to the minimum (A) and maximum (B) fluorescence polarization values, errors are given as standard deviations (SD). The data were fitted with the Hill-Langmuir equation (equation 2) where the dissociation constant (K_{D}) corresponds to the protein concentration [HT] at which half-polarization was measured. The Hill coefficient n was determined from the slope, whereat HaloTag proteins presenting one binding site, the Hill coefficient should be ideally equal to 1 for accurate K_{D} determination.

$$FP = A + \frac{{( B - A )}^{n}}{1 + {( \frac{{K}_{D}}{[ HT ]} )}^{n}} \qquad ( 2 )$$

FP: fluorescence polarization [mFP], A: min. FP, B: max. FP, [HT]: HaloTag protein concentration [mol/L], n: Hill coefficient, K_{D}: dissociation constant [mol/L].

Statistical analysis of presented K_{D}-values was aimed by performing the FP assay at least in three individual replicates (xₙ≤3). Mean K_{D}-values and the SD are presented.

Kinetics Measurements and pH-Dependency

Binding kinetics were measured by tracking the fluorescence polarization in a black flat bottom 380-well plate (20 μL) measured on a microplate reader (Spark20-Tecan) as previously explained for titration. The fluorescent nrHTL (50 nM) were spiked to HT7 protein (0.5 μM) and the fluorescence polarization was measured in techn. triplicates every 10 s by exciting the TMR at 535±12.5 nm and recording the emission at 595±17.5 nm. Gain was optimum at 56%.

To assess the pH-dependency of both FP experiments presented, 100 μL HT7 protein in activity buffer was dialysed in 200 mL SPG-Buffer (Jena Bioscience) at different pH-values ranging from 6.0 to 8.0 in dialysis units (Slide-A-Lyzer® MINI Dialysis Unit, 7,000 MWCO) over-night at 4° C. It was supplemented with 1% (w/v) BSA, the pH-values were evaluated using a SevenCompact pH-meter S220 device and eventually adjusted using 2 N HCl or NaOH.

Stopped-flow kinetics were measured at a BioLogic SFM-400 instrument (BioLogic Science Instruments). 4 μM HaloTag protein was mixed in a 1:1 (v/v) ratio with 1 μM fluorophore-nrHTL in activity buffer. The fluorescence intensity and the fluorescence polarization were measured over 300 s at 0.1 ms timepoints. Background data for fluorophore only were subtracted and at least 8 techn. replicates were averaged.

All kinetic measurements were performed at 37° C. For both experimental set-ups the time-dependent FP data were normalized to the highest polarization values (B) and fitted with the following non-linear equation yielding k₁ as the binding kinetic constant (on-rate):

$$FP = B + \frac{( 0 - \frac{B}{[ D ]} ) \cdot ( [ D ] \cdot ( [ D ] - [ HT ] ) ) \cdot {e}^{( [ D ] - [ HT ] ) \cdot {k}_{1} \cdot t}}{D \cdot {e}^{( ( [ D ] - [ HT ] ) \cdot {k}_{1} \cdot t ) - [ HT ]}} \qquad ( 3 )$$

FP: fluorescence polarization [mFP], B: max. FP, [D]: dye concentration, [HT]: HaloTag protein concentration, t: time [s], k1: on-rate [M⁻¹ s^{-1].}

In combination with experimentally evaluated K_{D}-values, the unbinding kinetic constants were calculated with the following equation:

k₋₁=K_{D}·k₁ (4)

K_{D}: dissociation constant [mol/L], k₁: on-rate [M⁻¹ s⁻¹], k₋₁: off-rate [s⁻¹].

### Fluorescence Turn-on Assay

The fluorophore-nrHTL probes (50 nM) were incubated in presence and absence of HT proteins (100 μM) for 30 min at 37° C. in activity buffer containing 1% (w/v) BSA in a black flat bottom 380 well plate (Greiner, 20 μL). Fluorescence emission scans were recorded, for example by exciting the SiR fluorophore at 605 +10 nm and measuring the emission intensity between 652 and 800 nm, on a microplate reader (Spark20-Tecan) with an automated gain of 50%. The data from three techn. replicates were averaged and the fluorescence turn-on was calculated by the quotient of fluorescence intensity in presence and absence of HaloTag protein at the emission maxima (e.g. in the case of SiR at 670 nm). The errors were calculated through standard error propagation from replicate's standard deviations. Reference experiments with corresponding fluorophore-HTLs were made in parallel and used to normalize the fluorescence emission spectra to 1.

### Water-Dioxane Titration

Solutions of 5 μM fluorophore-nrHTL were prepared in 10/90 to 80/20 (v/v) water-dioxane mixtures (dry, Acros) in transparent flat-bottom polypropylene 96-well plates (Greiner, Chimney Well). Absorbance spectra between 400 and 750 nm were recorded in a microplate reader (Spark20-Tecan) with 2 nm step size. The maximal absorbance at 646 nm was blank corrected, normalized to the max. and min. absorbance and plotted against the corresponding dielectric constants ε_{R}. Data were fitted with the following sigmoidal function delivering D₅₀-values corresponding to the dielectric constant at half-maximal absorbance.

$$Abs = \frac{A + ( B - A )}{( 1 + {10}^{( D 50 - {ε}_{R} ) \cdot n} )} \qquad ( 5 )$$

Abs: absorbance [AU], A: min. abs., B: max. abs., ε_{R}: dielectric constant, n: slope, D₅₀: ε_{R} at half-maximal absorbance.

TABLE 3
Composition of buffers used for in-vitro characterization.

| Buffer | Composition |
|---|---|
| TAE-Buffer | 5 mM Na₂EDTA, 40 mM Tris, 20 mM AcOH |
| Activity buffer | 50 mM HEPES, 50 mM NaCl, pH 7.3 |
| Extraction buffer | 50 mM KH₂PO₄, 300 mM NaCl, 5 mM imidazole, |
| | pH 8.0 |
| His wash buffer | 50 mM KH₂PO₄, 300 mM NaCl, 10 mM imidazole, |
| | pH 7.5 |
| His elution buffer | 50 mM KH₂PO₄, 300 mM NaCl, 500 mM imidazole, |
| | pH 7.5 |
| Laemmli Buffer | 31.5 mM Tris-HCl, pH 6.8, 10% glycerol, 1% SDS, |
| | 0.005% Bromophenol Blue |

Buffers were prepared in Mili-Q ® water.

### Cell Biology and In-Cellulo Experiments

### Cell Culture and Transient Transfection

U-2 OS cell lines were maintained in T-75 flasks (Greiner) in high-glucose Dulbecco's Modified Eagle Medium (DMEM GlutaMAX™, phenol-red, Gibco). Growth medium was supplemented with 10% (v/v) fetal calf serum (FCS) and cells were stored in a humidified tissue culture incubator at 37° C. and 5% CO₂. They were passaged using phosphate buffered saline (PBS, pH 7.4, Gibco) and TrypLE™ Select Enzyme (1×, phenol-red free, Gibco) every 2-3 days and regularly tested for mycoplasma contamination. U-2 OS Flp-In™ T-REX™ cells were used from commercial sources (ThermoFisherScientific) while the same cell lines stably expressing HaloTag7-SNAP-NLS, H₂B-HaloTag8 and Tomm20-HaloTag8 were generated and kindly provided by Dr. Birgit Koch or Dr. Michelle Frei. For staining and subsequent confocal microscopy imaging, 1.0 to 1.5×10⁵ cells per well were seeded into tissue culture treated CellCarrier-96 black plates with an optically clear glass-bottom (PerkinElmer). Cell titter were determined by counting detached cells in a fluidlab R-300 handheld cell counter.

Transient transfection was performed using Lipofectamine 3000® reagent (ThermoFisherScientific) according to the manufacturer's protocol (amount per 96-well given): To generate DNA-lipoplexes, plasmid DNA (0.1 μg) and 0.2 μL P300 reagent were mixed in 10 μL Reduced Serum Media Opti-MEM™ I (Gibco). In parallel, 0.2 μL Lipofectamine 3000® reagent was diluted in 10 μL Opti-MEM™. After a short incubation time the two solutions were mixed in a 1:1 ratio and incubated for at least 15 min at rt. Finally, 20 μL transfection mixture was added on top of the cells, which were seeded 16 to 18 h prior to transfection as described earlier, and mixed gently. The medium was changed 14 h after transfection back to regular growth medium, supplemented with 0.1 mg/mL doxycycline to induce protein expression.

### Staining, Fixation and Permeabilization

Live-cell staining was performed 22 h after seeding or transfection. Fluorophore-nrHTL were applied in imaging medium (DMEM GlutaMAX™, 10% FCS, phenol-red free, Gibco) at 10 nM to 1 μM concentrations for 16 h at 37° C., keeping the DMSO concentration below 1%.

After PBS wash, cells were fixed eventually using 4% (v/v) cell-culture grade paraformaldehyde solution (PFA, Electron Microscopy Sciences) in PBS for 20 min at room-temperature. The cells were subsequently washed with PBS and permeablize with 0.5% (v/v) Triton X-100 (Roth) in PBS (10 min, rt). Removal of the detergent was accomplished by washing three times with 3% BSA in PBS w/v). Cells were stored in PBS for maximum 1 week at 4° C. and stained with 500 nM fluorescent probes in activity buffer at room-temperature for 2 h prior to imaging.

### Confocal Fluorescence Microscopy

Fixed and live cell images were acquired by fluorescence confocal microscopy imaging on a Leica DMi8 microscope (Leica Microsystems) equipped with a Leica TCS SP8 X scanhead and a SuperK white light laser. A HC PL APO 20×/0.75 dry objective or a HC PL APO 40.0×/1.10 ater objective were used in combination with hybrid detectors (HyD). In case of potential spectral overlap (e.g. JF₅₈₅ and SiR), sequential images were taken to avoid cross-talks. Laser intensities between 0.8% and 12% were used while all other settings were kept constant. Living cells were maintained in a CO₂ (5%) and temperature-controlled (37° C.) incubator (Life Imaging Services).

Pulse-chase experiments were carried out by addition or replacement of the staining solution directly under the microscope and recording of z-stacks over time. Photobleaching was performed using the FRAP module of the Leica DMi8 microscope, a TMP detector and the following bleaching sequence: single nuclei were bleached three times in a row with 100% laser power for 3.4 s in a circular ROI. Afterwards, single-stack images were taken every 10 s for 1 min. This sequence was repeated 10 times consecutively and the fluorescence intensity was quantified using the LAX software.

In-cellulo signal-to-noise ratios (S/N) were extracted from the acquired images by analyzing them with the ImageJ Fiji software as follows: the mean signal intensity of a circular ROI within a labeled nucleus was divided by the mean signal intensity of a circular ROI adjacent to the nucleus (cytosolic background signal).

### Super-Resolution Fluorescence Microscopy

For STED microscopy U-2 OS CRISPR-Vimentin-HaloTag7 cells seeded on glass coverslips were kindly provided by in-house collaborators from the Optical Microscopy facility (MPImF, Heidelberg). They were incubated in imaging medium that contained 0.5 μM SiR-nrHTL or SIR-HTL for 30 min at 37° C. Living cells were imaged using an Abberior STED 775/595/RESOLFT QUAD scanning microscope (Abberior Instruments GmbH) equipped with a UPlanSApo 100×/1.4 oil immersion objective lens. SiR was excited at 640 nm (2.0%) and detected at 655 to 700 nm. STED-images were recorded by using a STED-laser at 755 nm (15.0%), a pixel dwell time of 15 μs and a pixel size of 30 nm. Quantitative analysis of single vimentin fibrils was performed with ImageJ by measuring plot profile scans which are represented as Gaussian distribution.

HT-PAINT microscopy was carried out by Sebastian Strauss (AG Jungmann, MPI for Biochemistry, Martinsried) as explained in the following: 3×10⁵ U-2 OS CRISPR-NUP96-Halo cells per well were seeded into 8-well chambered coverslip (ibidi) and grown overnight. The cells were washed with PBS once and fixed with 2.4% PFA in PBS for 30 min at room-temperature. After fixation, the cells were rinsed three times with PBS, permeabilized with 0.25% Triton-X-100 (5 min) and blocked with BSA-PBS (3% w/v, 30 min). Finally, nrHTL were added at a concentration of 2 nM in PBS pH 7.2.

Image acquisition was carried out on an inverted microscope (Nikon Instruments, Eclipse Ti2) with the Perfect Focus System, applying an objective-type TIRF configuration equipped with an oil-immersion objective (Nikon Instruments, Apo SR TIRF 100×, NA 1.49, Oil) and 561 nm as well as 642 nm laser-lines (MPB Communications Inc., 2 W, DPSS-system) were used for excitation. The laser beams were passed through cleanup filters (Chroma Technology, ZET561/10, ZET 640/10) and coupled into the microscope objective using a beam splitter (Chroma Technology, ZT561rdc, ZT640rdc). Fluorescence light was spectrally filtered with an emission filter (Chroma Technology, ET600/50m and ET700/75m) and imaged on a sCMOS camera (Andor, Zyla 4.2 Plus) without further magnification, resulting in an effective pixel size of 130 nm (after 2×2 binning). Images were acquired choosing a region of interest with the size of 512×512 pixels. The detailed imaging parameters are described in Table 4.

The raw data was reconstructed and post-processed using the ‘Picasso’ software package (Schnitzbauer, J. et al., Nat Protoc 12, 1198-1228, 2017). Drift correction was performed using gold nanoparticles as fiducial markers.

TABLE 4
Microscope settings used for HT-PAINT imaging

| | λₑₓₜ | Laser power | # | Integration time |
|---|---|---|---|---|
| Dye | [nm] | [mW] | Frames | [ms] |
| TMR | 561 | 10 | 30,000 | 100 |
| SiR | 642 | 30 | 15,000 | 200 |

### 6.1.1 General Procedures

General Procedure A: Alkyl Iodination (mesylation and Finkelstein Reaction)

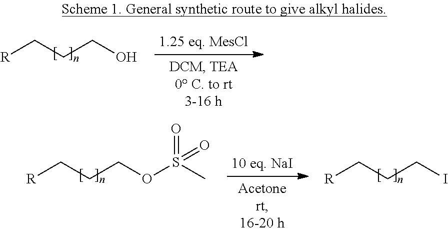

A heat-dried round-bottom flaks was charged with dry DCM (5 mL/mmol alkyl alcohol) and 1.5 eq. triethylamine (TEA). 1 eq. alkyl alcohol was added and the mixture was cooled to 0° C. Afterwards, 1.25 eq. methanesulfonylchloride was added dropwise under continued stirring and the solution was left stirring for 1 h at 0° C. and 3 h at room-temperature. The reaction was monitored by TLC and if necessary carried out for 12 to 16 h over-night.

After full conversion of the starting material 10 mL/mmol 10% NH₄Cl solution was added and the aq. layer was extracted three times with 4 Veq. DCM. The combined organic phases were washed with 2 Veq. brine and dried over MgSO₄. Filtration and evaporation of the solvent under reduced pressure gave the crude compound. Final purification of the desired alkyl methanesulfonate was performed by flash column chromatography (SiO₂. Typical gradient: 0 to 2% MeOH in DCM for 6 CV).

1 eq. Methanesulfonate compound and 10 eq. NaI were mixed in 1.4 mL/mmol acetone in a round-bottom flask. The emulsion was stirred at room-temperature over-night. After 16 to 20 h large quantities of a yellow-brown precipitate were observed.

The residual solvent was removed under reduced pressure and the remaining solid was dissolved in 10 mL/mmol DCM and H₂O each. The aq. layer was extracted twice with 2 Veq. DCM. Afterwards the org. layers where combined and a pale-purple discolouration was removed upon washing with 100 mL sat. Na₂S₂O₃. Subsequent brine wash, drying over MgSO₄, filtration and concentration in vacuo gave the crude compound. Final purification by flash column chromatography (SiO₂. Typical gradient: 5 to 30% EtOAc in n-hexane in 6 CV) afforded the desired compound.

### General Procedure B: Williamson Ether Synthesis

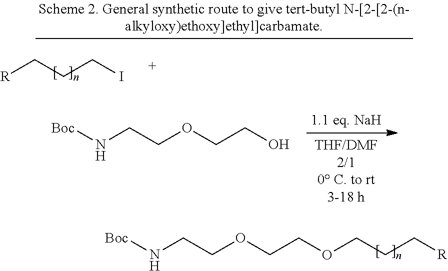

Ether synthesis reaction was adopted according to Takashima et al. 2019. A reaction tube was charged with 2 mL/mmol of a 2/1 ratio (v/v) of dry THF and DMF under Schlenk conditions. 1 eq. tert-Butyl(2-(2-hydroxyethoxy)ethyl)carbamate (BocNH-PEG₂-OH) was added and dissolved under vigorous stirring. The mixture was cooled to 0° C. and 1.1 eq. NaH was added portion-wise. The evolving gas was released carefully and the mixture was left stirring at 0° C. for 30 min under an inert gas atmosphere. Afterwards, 1.4 eq. alkyl halide was added directly into the suspension. The mixture was warmed to room-temperature and left stirring for 3 h to 18 h while the reaction progress was controlled by TLC.

Upon full conversion of the alkyl halide species 10 mL/mmol 10% aq. NH₄Cl and EtOAc each were added to stop the reaction. The aq. layer was extracted three times with 10 mL/mmol EtOAc. The org. layers were combined and washed with brine once and three times with 10% LiCl to remove residual DMF. Afterwards, it was dried over MgSO₄, filtered and the solvent was removed under reduced pressure. Purification was performed by flash column chromatography (SiO₂. Typical gradient: 20 to 50% EtOAc in n-hexane in 8 CV) to afford the desired BocNH-PEG₂-Cₙ-R compounds.

### General Procedure C: Boc Protecting Group Cleavage

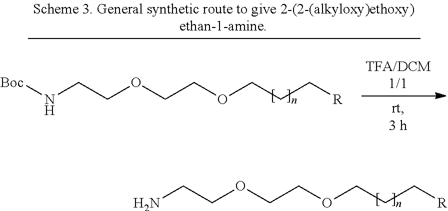

Removal of Boc protecting groups was reached under acidic conditions. Therefore, the protected compounds were dissolved in 3 mL/mmol of a 1/1 (v/v) mixture of TFA in dry DCM.

It was stirred at room-temperature for at least 3 h and the reaction progress was followed by TLC.

Afterwards the solvent was removed under reduced pressure, co-evaporated three times with 1 Veq. DCM at 700 mbar and 40° C. and finally dried under a stream of N₂ for at least 30 min to collect H₂N-PEG₂-Cn-R compounds, which were used without further purification.

6.1.2 Boc-NH-PEG₂-C₄/₅-Methylsuflonamide/Triflamide/Acetamide (9-12)

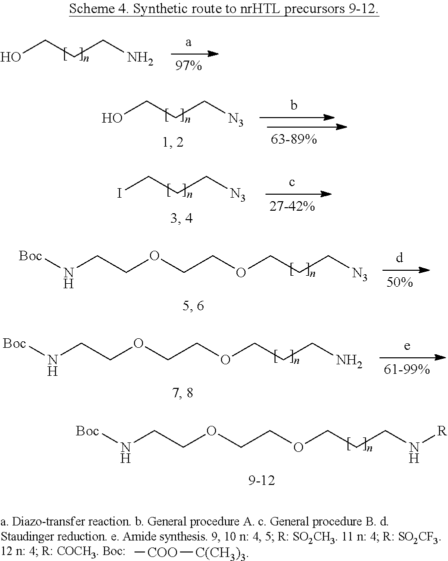

a. 4-Azido-1-butanol (1)/5-azido-1-pentanol (2)

The diazo-transfer reagent imidazole-1-sulfonyl azide hydrochloride was synthesized following the protocol of Goddard-Borger et al. 2007. NaN₃ (6.5 g, 100 mmol, 1 eq.) was filled into a heat-dried Schlenk-flask and suspended in 100 mL dry MeCN under a flow of argon gas. It was cooled to 0° C. and sulfuryl chloride (8.1 mL, 100 mmol, 1 eq.) was added drop-wise. The mixture was left stirring over-night at room-temperature. Afterwards imidazole (12.9 g, 190 mmol, 1.9 eq.) was added portion-wise at 0° C. The resulting slurry was left stirring at rt for 3 h and diluted with 200 mL EtOAc. The organic layer was washed twice with 200 mL H₂O and sat. NaHCO₃ solution each, dried over MgSO₄ and filtrated. Meanwhile, acetyl chloride (10.7 mL, 150 mmol, 1.5 eq.) was added drop-wise to 37.5 mL ice-cold dry ethanol (EtOH) to obtain 4 M HCl in EtOH. Upon dropwise addition onto the filtrate solution stirring at 0° C. the formation of colourless needles was observed. Filtration and washing with 3×100 mL ice-cold EtOAc delivered 15.4 g (88.9 mmol, 74%) imidazole-1-sulfonyl azide·HCl as a white powder. ¹H NMR (400 MHZ, CDCl₃): δ7.47 (s, 1H), 5.97 (s, 1H), 5.64 (s, 1H).

4-Amino-1-butanol (1.2 mL, 12.9 mmol, 1 eq.) or 5-amino-1-pentanol (3.1 g, 30 mmol, 1 eq.), 2.25 eq. K₂CO₃ and 1 mol % Cu(II)SO₄.5 H₂O were suspended in a heat-dried Schlenk-flask in 5 mL/mmol dry MeOH under a stream of argon gas. The solution was cooled to 0° C. and 1.2 eq. imidazole-1-sulfonyl azide·HCl was added portion-wise. The mixture was left stirring for 12 h at rt, concentrated under reduced pressure and acidified with some drops of conc. HCl. The remaining solid was taken up in 200 mL EtOAc, washed with 100 mL H₂O and brine each, dried over MgSO₄, filtrated and evaporated to afford the crude products as a pale-yellow oil. 1.5 mg 1 (13 mmol) was collected after flash column chromatography on a 25 g SiO₂ column (20% EtOAc in n-hexane for 14 CV). 3.3 mg 2 (25 mmol) was afforded by flash column chromatography on a 40 g SiO₂ column (5 to 70% EtOAc in n-hexane for 8 CV).

4-Azido-1-butanol (1)—Yield: 97%. ¹H NMR (400 MHZ, CDCl₃): δ3.57 (t, 2H), 3.25 (t, 2H), 1.58 (m, 4H). ¹³C NMR (101 MHZ, CDCl₃): δ61.84, 51.16, 29.48, 25.20.

5-Azido-1-pentanol (2)—Yield: 99%. ¹H NMR (400 MHZ, CDCl₃): δ3.63 (t, 2H), 3.26 (t, 2H), 1.59 (m, 4H), 1.44 (m, 2H). ¹³C NMR (101 MHZ, CDCl₃): δ62.24, 51.38, 32.10, 28.62, 22.98. HRMS (m/z): [M +H]+calcd. for C₅H₁₂N₃O⁺, 130.0975; found, 130.0974.

b. 4-Azido-1-iodobutane (3)/5-azido-1-iodopentane (4)

4-Azido-1-butanol 1 (1.5 g, 13 mmol) or 5-azido-1-pentanol 2 (3 g, 23.2 mmol) were treated as described in general procedure A. Almost full consumption of the starting materials was observed after stirring for 1 h at 0° C. and 3 h at room-temperature, respectively. Aq. work-up and purification over a 25 g silica column (0 to 3% MeOH in DCM for 9 CV) delivered 1.8 g 4-azidobutyl-1-methansulfonate (9.4 mmol, 72%) and 4.5 g 5-azidopentyl-1-methansulfonate (22 mmol, 95%) as yellow oils, respectively.

4-Azidobutyl-1-methansulfonate—¹H NMR (400 MHZ, CDCl₃): δ4.20 (t, 2H), 3.29 (t, 2H), 2.95 (s, 3H), 1.79 (m, 2H), 1.67 (m, 2H). ¹³C NMR (101 MHZ, CDCl₃): δ69.28, 50.37, 37.37, 26.33, 25.06. HRMS (m/z): [M+Na]⁺ calcd. for C₅H₁₁N₃O₃SNa⁺, 216.0413; found, 216.0413.

5-Azidopentyl-1-methansulfonate—¹H NMR (400 MHZ, CDCl₃): δ4.22 (t, 2H), 3.29 (t, 2H), 3.00 (s, 3H), 1.78 (dq, 2H), 1.63 (ddt, 4H). ¹³C NMR (101 MHZ, CDCl₃): δ69.70, 51.20, 37.44, 28.77, 28.37, 22.84. HRMS (m/z): [M+H]⁺ calcd. for C₆H₁₆N₃O₃S⁺, 208.0750; found, 208.0753.

Subsequent Finkelstein reaction was carried out with 1 eq. 4-azidobutyl-1-methanesulfonate (1.8 g, 9.4 mmol) or 5-azidopentyl-1-methanesulfonate (3.3 g, 16 mmol) according to general procedure A. Aq. work-up and purification on a 25 g SiO₂ column (5 to 30% EtOAc in n-hexane for 6 CV) gave 1.84 g 3 (8.2 mmol, 88%) or 3.45 g 4 (14.5 mmol, 91%) as colourless liquids.

4-Azido-1-iodobutane (3)—Yield: 63% over two steps. ¹H NMR (400 MHZ, CDCl₃): δ3.31 (q, 2H), 3.19 (q, 2H), 1.90 (m, 2H), 1.70 (tt, 2H). ¹³C NMR (101 MHZ, CDCl₃): δ50.43, 29.81, 30.51, 5.83.

5-Azido-1-iodopentane (4)—Yield: 89% over two steps. ¹H NMR (400 MHZ, CDCl₃): δ3.29 (t, 2H), 3.19 (t, 2H), 1.85 (p, 2H), 1.54 (m, 4H). ¹³C NMR (101 MHZ, CDCl₃): δ51.31, 33.02, 27.97, 27.79, 6.51.

c. Boc-NH-PEG₂-C₄-N₃ (5)/Boc-NH-PEG₂-C₅-N₃ (6)

Following general procedure B, 1 eq. Boc-NH-PEG₂-OH was taken up in 2 mL/mmol THF/DMF 2/1 and left reacting with 1.1 eq. NaH at 0° C. for 30 min. Immediately after the addition of 1.4 eq. 4-azido-1-iodobutane (1.84 g, 8.2 mmol) or 5-azido-1-iodopentane (1.63 g, 6.82 mmol) the formation of a white solid was observed. The mixture was warmed to rt and after 3 h stirring the complete conversion of the starting materials was observed. Aq. work-up and purification on a 25 g SiO₂ column (20 to 50% EtOAc in n-hexane in 8 CV) was used to afford the desired products. 500 mg 5 (1.65 mmol) and 650 mg 6 (2.1 mmol) were collected as colourless liquids.

tert-Butyl N-[2-[2-(4-azidobutyloxy)ethoxy]ethyl]carbamate (Boc-NH-PEG₂-C₄-N₃, 5)—Yield: 27%. ¹H NMR (400 MHZ, CDCl₃): δ4.99 (s, NH), 3.55 (m, 8H), 3.30 (t, 4H), 1.67 (m, 4H), 1.43 (s, 9H). ¹³C NMR (101 MHZ, CDCl₃): δ156.05, 79.24, 70.70, 70.28, 51.32, 40.36, 31.65, 28.50 (3C), 26.81, 25.80, 14.26. HRMS (m/z): [M+Na]⁺ calcd. for C₁₃H₂₆N₄O₄Na⁺, 325.1846; found, 325.1848.

tert-Butyl N-[2-[2-(5-azidopentyloxy)ethoxy]ethyl]carbamate (Boc-NH-PEG₂-C₅-N₃, 6)—Yield: 42%. ¹H NMR (400 MHZ, CDCl₃): δ5.01 (s, NH), 3.56 (m, 6H), 3.45 (d, 2H), 3.27 (m, 4H), 1.61 (m, 4H), 1.43 (s, 9H), 1.42 (m, 2H). ¹³C NMR (101 MHZ, CDCl₃): δ156.10, 79.26, 71.20, 70.33, 70.16, 51.45, 40.40, 29.21, 28.77, 28.51 (3C), 21.17, 14.30. HRMS (m/z): [M+H]⁺ calcd. for C₁₄H₂₈N₄O₄Na⁺, 339.2003; found, 339.2004.

d. Boc-NH-PEG₂-C₄-NH₂ (7)/Boc-NH-PEG₂-C₅-NH₂ (8)

For Staudinger reduction 500 mg alkyl azide compounds 5 (1.65 mmol) or 6 (1.56 mmol) were dissolved in 4 mL/g dry THF in a dry round-bottom flask under an argon atmosphere. 1.5 eq. PPh₃ was added and the mixture was left stirring for 24 h air-excluded. Afterwards, 10 eq. H₂O were added and the mixture was left stirring for 24 h. The solution was concentrated in vacuo and purified on a 12 g silica column with 10% MeOH and 0.5% TEA in DCM for 8 CV. For both reactions 230 mg of a pale-yellow gum were collected as 7 (0.83 mmol) and 8 (0.79 mmol).

tert-Butyl N-[2-[2-(4-aminobutyloxy)ethoxy]ethyl]carbamate (Boc-NH-PEG₂-C₄-NH₂, 7)—Yield: 50%. ¹H NMR (400 MHZ, MeOD): δ4.58 (NH₂), 3.61 (s, 4H), 3.52 (dt, 4H), 3.22 (t, 2H), 2.97 (t, 2H), 1.74 (m, 4H), 1.43 (s, 9H). ¹³C NMR (101 MHZ, MeOD): δ157.30, 78.91, 70.27, 69.92, 69.86, 48.57, 49.92, 39.49, 27.47 (3C), 26.51, 24.85. HRMS (m/z): [M+H]⁺ calcd. for C₁₃H₂₉N₂O₄⁺, 277.2122; found, 277.2119.

tert-Butyl N-[2-[2-(5-aminopentyloxy)ethoxy]ethyl]carbamate (Boc-NH-PEG₂-C₅-NH₂, 8)—Yield: 50%. ¹H NMR (400 MHZ, MeOD): δ3.58 (s, 4H), 3.54 (m, 4H), 3.22 (t, 2H), 2.67 (t, 2H), 1.58 (m, 2H), 1.44 (s, 9H), 1.46 (m, 4H). ¹³C NMR (101 MHZ, MeOD): δ157.60, 79.20, 71.39, 70.35, 70.18, 41.53, 40.39, 32.51, 29.63, 27.91 (3C), 23.64. HRMS (m/z): [M+H]⁺ calcd. for C₁₄H₃₁N₂O₄⁺, 291.2278; found, 291.2275.

e. Terminal (sulfon)amide Synthesis (9-12)

1 eq. alkylamine 7 or 8 was dissolved in 6 mL/mmol dry DCM. Afterwards 1.5 eq. TEA was added and the mixture was cooled to 0° C. and stirred vigorously under an argon atmosphere. 1.25 eq. Methanesulfonyl chloride was added dropwise at 0° C. to the solution of 7 or 8. In similar reactions 1.25 eq. trifluormethanesulfonyl chloride or 1.0 eq. acetyl chloride were added to 7. The mixture was stirred for 1 h at 0° C., warmed to room-temperature and left stirring over-night at room-temperature.

For aq. work-up the reaction mixture was diluted with 2 Veq. DCM, 1 Veq. 1 N HCl was added and the aq. phase was extracted twice with 1 Veq. DCM. The combined organic layers were washed once with 1 Veq. brine and dried over MgSO₄. Filtration and removal of the solvent under reduced pressure delivered the crude product. Final purification was reached by flash column chromatography (12 g SiO₂) and the desired compounds were afforded in 61 to 99% yields.

TABLE 5
Purification conditions and yields of nrHTL precursors

| Product | Gradient | Yield [%] |
|---|---|---|
| 9 | 0 to 10% MeOH in DCM in 13 CV | 61 |
| 10 | 20 to 80% EtOAc in n-hexane in 8 CV | 64 |
| 11 | 40 to 70% EtOAc in n-hexane in 8 CV | 99 |
| 12 | 0 to 6% MeOH in DCM in 8 CV | 80 |

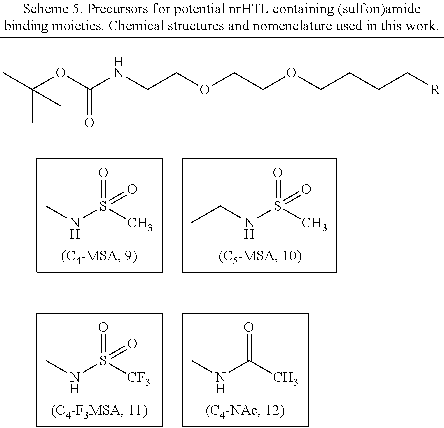

tert-Butyl (2-(2-((4-(methylsulfonamino)-butyl)oxy)ethoxy)ethylcarbamate (BocNH-PEG₂-C₄-MSA, 9)—Yield: 61%. ¹H NMR (400 MHZ, CDCl₃): δ3.55 (m, 8H), 3.30 (t, 2H), 3.15 (t, 2H), 2.93 (s, 3H), 1.69 (m, 4H), 1.44 (s, 9H). ¹³C NMR (101 MHZ, CDCl₃): δ157.06, 78.69, 70.40, 69.84, 69.77, 69.65, 42.45, 38.32, 29.37, 27.36 (3C), 26.55, 26.36. HRMS (m/z): [M+Na]⁺ calcd. for C₁₅H₃₀N₂O₆SNa⁺, 377.1717; found, 377.1714.

tert-Butyl (2-(2-((5-(methylsulfonamino)pentyl)-oxy)ethoxy)ethylcarbamate (BocNH-PEG₂-C₅-MSA, 10)—Yield: 64%. ¹H NMR (400 MHZ, CDCl₃): δ3.59 (m, 6H), 3.33 (t, 2H), 3.34 (q, 2H), 3.16 (q, 2H), 2.94 (s, 3H), 1.65 (m, 4H), 1,46 (m, 2H), 1.44 (s, 9H). ¹³C NMR (101 MHZ, CDCl₃): δ 156.06, 79.25, 77.26, 70.98, 70.22, 70.06, 43.15, 40.28, 29.83, 28.91, 28.44 (3C), 23.19, 21.3. HRMS (m/z): [M+Na]⁺ calcd. for C₁₆H₃₂N₂O₆SNa⁺, 369.2051; found, 369.2051.

tert-Butyl (2-(2-((4-(trifluoromethylsulfonamino)-butyl)oxy)ethoxy)ethylcarbamate (BocNH-PEG₂-C₄-F₃MSA, 11)—Yield: 99%. ¹H NMR (400 MHZ, CDCl₃): δ3.56 (m, 4H), 3.52 (m, 4H), 3.30 (m, 4H), 1.74 (m, 4H), 1.43 (s, 9H). ¹³C NMR (101 MHZ, CDCl₃): δ156.16, 121.35 (q, CF₃), 77.26, 70.97, 70.16 (2C), 69.80, 44.16 (2C), 28.37 (3C), 28.99 (2C). ¹⁹F NMR (376 MHZ, CDCl₃): δ-77.23 (s, 3F). HRMS (m/z): [M+Na]⁺ calcd. for C₁₆H₂₉N₂O₆SF₃Na⁺, 431.1434; found, 431.1431.

tert-Butyl (2-(2-((4-(acetamidobutyl)oxy)-ethoxy)ethylcarbamate (BocNH-PEG₂-C₄-NAc, 12)—Yield: 80%. ¹H NMR (400 MHZ, CDCl₃): δ3.54 (m, 6H), 3.44 (t, 2H), 3.25 (p, 2H), 3.19 (t, 2H), 1.92 (s, 3H), 1.57 (tdd, 4H), 1.43 (s, 9H). ¹³C NMR (101 MHZ, CDCl₃): δ170.51, 156.12, 79.85, 70.13, 70.00, 50.58, 40.27, 39.32, 28.38 (3C), 26.89, 26.21(2C), 23.14. HRMS (m/z): [M+Na]⁺ calcd. for C₁₅H₃₀N₂O₅Na⁺, 341.2047; found, 341.2042.

6.1.3 Boc-NH-PEG₂-C₅-Methylamine(NBoc) (15)

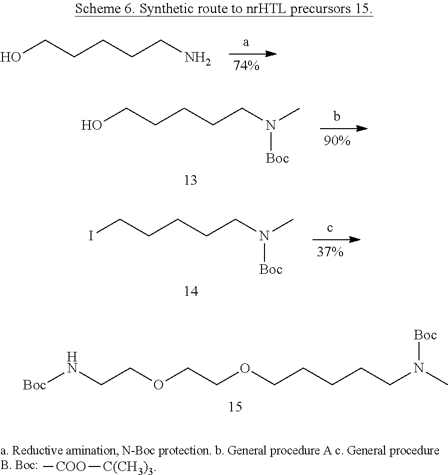

a. tert-Butyl (5-hydroxypentyl)(methyl)carbamate (13)

Reductive amination was carried out in a two-step reaction according to Ji et al. 2019 (US2019192668A1). First, 1 eq. 5-aminopentane-1-ol (5.3 g, 49 mmol) was dissolved in 4 eq. ethyl formate (15.7 mL, 194 mmol). The mixture was heated to 90° C. and stirred under reflux conditions for 6 h, cooled to room-temperature and concentrated in vacuo. The crude compound was purified on a 40 g silica-column with 2 to 8% MeOH in DCM over 14 CV and 4.8 g N-(5-hydroxypentyl) formamide (36.4 mmol, 74%) was collected as a colourless liquid. ¹H NMR (400 MHZ, MeOD): δ8.02 (s, 1H), 3.55 (t, 2H), 3.22 (t, 2H), 1.55 (m, 4H), 1.41 (m, 2H). ¹³C NMR (101 MHZ, MeOD): δ163.72, 62.71, 38.90, 33.18, 30.13, 24,20. HRMS (m/z): [M+Na]⁺ calcd. for C₆H₁₂NO₂Na⁺, 154.0838; found, 154.0837.

Next, a flame-dried 250 mL round-bottom flaks was charged with 694 mg LiAlH₄ (18.3 mmol, 1.2 eq.) and 22 mL dry THF. The mixture was cooled to 0° C. 1 eq. N-(5-hydroxypentyl) formamide (2 g, 15.2 mmol) from the previous reaction was dissolved in 5 mL dry THF and added drop-wise under strong stirring and continuous cooling. A gray precipitate formed rapidly. After complete addition of the reagents, the mixture was heated to 80° C. and stirred under reflux conditions for 2 h. The reaction was stopped by the addition of 2 mL 15% NaOH and some drops of water. It was dried over MgSO₄ the precipitated was removed by filtration and washed extensively with THF. 1.8 g crude 5(-methylamino)pentan-1-ol was collected as a colourless oil. A small proportion was purified over 12 g SiO₂ with 6 to 20% MeOH in DCM over 8 CV. Residual silica gel was removed by filtration over a short plug of Celite® and the desired compound was afforded in 97% yield. ¹H NMR (400 MHZ, MeOD): δ3.47 (t, 3H), 3.27 (s, 3H), 3.16 (q, 2H), 1.47 (m, 4H), 1.33 (m, 2H). HRMS (m/z): [M+H]⁺ calcd. for C₆H₁₂NO, 118.1226; found, 118.1227.

Finally, 1 eq. purified 5(-methylamino)pentan-1-ol (300 mg, 2.6 mmol) was dissolved in 4 mL dry MeOH and 550 UL di-tert-butyl dicarbonate (2.6 mmol, 1 eq.) were added. The mixture was stirred for 12 h at room-temperature, concentrated and purified by silica flash column chromatography (12 g SiO₂) with 30 to 50% EtOAc in n-hexane over 12 CV. The title compound 13 was collected in 95% yield (650 mg, 2.3 mmol) and appeared as a colourless liquid.

tert-Butyl (5-hydroxypentyl)(methyl)carbamate (13)—Yield: 75% over three steps. ¹H NMR (400 MHZ, CDCl₃): δ3.63 (t, 2H), 3.30 (t, 2H), 2.82 (s, 3H), 1.56 (m, 4H), 1.44 (s, 9H), 1.34 (m, 2H). ¹³C NMR (101 MHZ, CDCl₃): δ156.04, 79.33, 62.87, 34.31, 32.45, 28.59 (4C), 22.85. HRMS (m/z): [M+Na]⁺ calcd. for C₁₁H₂₃NO₃Na⁺, 240.1570; found, 240.1569.

b. tert-Butyl (5-iodopentyl)(methyl)carbamate (14)

Mesylation of 500 mg 13 (2.3 mmol) was carried out as described in general procedure A. After flash column purification (12 g SiO₂, 0 to 5% MeOH in DCM, 15 CV) 640 mg 5-((tert-butoxycarbonyl)(methyl)amino)pentyl methanesulfonate (2.3 mmol, 93%) was received as a pale-yellow oil. ¹H NMR (400 MHZ, CDCl₃): δ4.22 (t, 2H), 3.21 (t, 2H), 2.99 (s, 3H), 2.82 (s, 3H), 1.76 (m, 2H), 1.54 (qd, 2H) 1.44 (s, 9H), 1.39 (m, 2H). HRMS (m/z): [M+Na]⁺ calcd. for C₁₂H₂₅NO₅SNa⁺, 318.1346; found, 318.1241.

Further, 300 mg 5-((tert-butoxycarbonyl)(methyl)amino)pentyl methanesulfonate (1 mmol) was used in a Finkelstein reaction as described in general procedure A. Flash column purification with 12 g SiO₂ (5 to 20% EtOAc in n-hexane, 6 CV) was performed to afford 240 mg the title compound 14 (0.74 mmol, 73%) which appears as a pale-orange liquid.

tert-Butyl (5-iodopentyl)(methyl)carbamate (14)—Yield: 90% over two steps. ¹H NMR (400 MHZ, CDCl₃): δ3.10 (q, 4H), 2.83 (s, 3H), 1.84 (p, 2H), 1.52 (m, 2H), 1.45 (s, 9H), 1.39 (m, 2H). HRMS (m/z): [M+Na]⁺ calcd. for C₁₁H₂₂NO₂INa⁺, 350.0587; found, 350.0589.

c. tert-Butyl N-[2-[2-(5-N-Boc-(methyl)aminopentyl)ethoxy]ethyl]carbamate (15)

According to general procedure B, 3 mL of a 2/1 mixture of dry THF and DMF and 500 mg tert-butyl (2-(2-hydroxyethoxy)ethyl)carbamate (2.4 mmol, 1 eq.) were mixed by vigorous stirring. The mixture was cooled to 0° C. and 102 mg NaH (2.55 mmol, 1.1 eq.) were added portion-wise. The emulsion was left stirring for 30 min at 0° C. under air-exclusion. Afterwards 860 mg 14 (2.9 mmol, 1.2 eq.) were added quickly and the reaction was left stirring for 18 h. The subsequent aq. work-up and column purification (12 mg SiO₂, 20 to 50% EtOAc/n-hexane, 8 CV) gave the title compound (360 mg, 0.89 mmol, colourless oil) in 37% yield.

tert-Butyl N-[2-[2-(5-N-Boc-(methyl)aminopentyl)ethoxy]ethyl]carbamate (15)—Yield: 37%. ¹H NMR (400 MHZ, CDCl₃): δ3.42 (m, 6H), 3.31 (t, 2H), 3.16 (t, 2H), 3.04 (t, 2H), 2.67 (s, 3H), 1.46 (m, 2H), 1.36 (m, 2H), 1.29 (dd, 18H), 1.18 (qd, 2H). ¹³C NMR (101 MHZ, CDCl₃): δ155.12, 155.94, 79.33, 79. 23, 71.45, 70.39, 70.34, 70.15, 48.72, 40.60, 34.21, 29.46, 28.60, 28.54, 27.71, 23.40. HRMS (m/z): [M+Na]⁺ calcd. for C₂₀H₄₀N₂O₆Na⁺, 427.2779; found, 427.2779.

6.1.4 Boc-NH-PEG₂-C₃₋₅-OTBS (18-20)/Boc-NH-PEG₂-C₄-OMe (21)

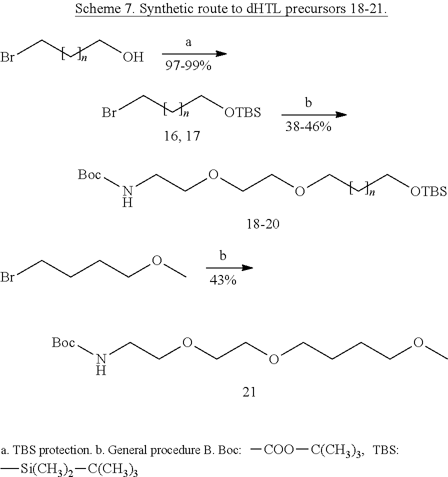

a. tert-Butyl (3-bromopropoxy)dimethylsilane (16)/tert-butyl (5-bromopentoxy)-dimethylsilane (17)

TBS protection of commercially available bromoalkyl alcohols was carried out in DCM with tert-butyl-chlordimethylsilan (TBDMS-Cl). Therefore, 1 eq. 1-bromopropan-3-ol (0.5 mL, 5.76 mmol) or 1-bromopentan-5-ol (1.5 g, 9 mmol) was dissolved in 1.75 mL/mmol dry DCM. 1.5 eq. imidazole was added and the mixture was cooled to 0° C. under vigorous stirring.

Finally, 1.1 eq. TBDMS-Cl was added portion-wise. The formation of a white precipitate was observed immediately. After 1 h stirring at room-temperature 1 mL/mmol 10% NH₄Cl was added to deactivated excess of TBDMS-Cl. The aq. layer was extracted with 5 mL/mmol DCM, the org. phases were combined and washed with brine once. It was dried over MgSO₄, filtrated and concentrated in vacuo. Purification of the crude product was reached on 25 g silica column with 0 to 30% EtOAc/n-hexane in 8 CV. The title compounds 16 were collected in 97% yield (1.42 g, 5.6 mmol) or 17 in 99% yield (2.5 g, 9.25 mmol) appearing as a colourless oil.

tert-Butyl (3-bromopropoxy)dimethylsilane (16)—Yield: 97%. ¹H NMR (400 MHZ, CDCl₃): δ3.73 (t, 2H), 3.51 (t, 2H), 2.03 (ddd, 2H), 0.89 (s, 9H), 0.06 (s, 6H). ¹³C NMR (101 MHZ, CDCl₃): δ60.55, 35.70, 30.78, 26.04 (3C), −5.24 (2C). HRMS (m/z): [M+H]⁺ calcd. for C₉H₂₁BrOSi⁺, 253.0618, 255.0598; found, 253.0618, 255.0597.

tert-Butyl (5-bromopentoxy)dimethylsilane (17)—Yield: 99%. ¹H NMR (400 MHZ, CDCl₃): δ3.61 (t, 2H), 3.41 (t, 2H), 1.88 (m, 2H), 1.52 (m, 4H), 0.89 (s, 9H), 0.05 (s, 6H). ¹³C NMR (101 MHZ, CDCl₃): δ62.99, 33.96, 32.76, 32.05, 26.01 (3C), 24.73, −5.15 (2C). HRMS (m/z): [M+H]⁺ calcd. for C₁₁H₂₅BrOSi⁺, 281.0931, 283.0911; found, 281.0933, 283.0913.

b. Boc-NH-PEG₂-C₃₋₅-OTBS (18-20)/Boc-NH-PEG₂-C₄-OMe (21)

The title compounds were synthesized according to general procedure B. Therefore, 1 mg 16 (3.95 mmol) or 2 g 17 (7.1 mmol) were used. Further, commercially available tert-butyl (3-bromobutoxy)dimethylsilane (2 mL, 7.2 mmol) or 4-methoxy-1-bromobutan (0.8 g, 4.8 mmol) were used. All reactions were completed overnight after 16 to 20 h. Subsequent aq. work-up and flash column purification (25 g SiO₂, 10 to 50% EtOAc in n-hexane, 10 CV) gave the desired compounds in 38 to 43% yield.

tert-Butyl (2,2,3,3-tetramethyl-4,8,11-trioxa-3-silatridecan-13-yl)carbamate (Boc-NH-PEG₂-C₃-OTBS, 18)—Yield: 40%. ¹H NMR (400 MHZ, CDCl₃): δ3.68 (t, 2H), 3.55 (m, 8H), 3.20 (q, 2H), 1.78 (p, 2H), 1.78 (s, 9H), 0.87 (s, 9H), 0.03 (s, 6H). HRMS (m/z): [M+H]⁺ calcd. for C₁₈H₃₉NO₅Si⁺, 378.2670; found, 378.2667.

tert-Butyl (2,2,3,3-tetramethyl-4,8,11-trioxa-3-silatetradecan-14-yl)carbamate (Boc-NH-PEG₂-C₄-OTBS, 19)—Yield: 38%. ¹H NMR (400 MHZ, CDCl₃): δ3.56 (m, 10H), 1.60 (m, 6H), 1.44 (s, 9H), 0.89 (s, 9H), 0.04 (s, 6H). HRMS (m/z): [M+Na]⁺ (ATBST) calcd. for C₁₃H₂₇NO₅Na, 300,1718; found 300, 1718.

tert-Butyl (2,2,3,3-tetramethyl-4,8,11-trioxa-3-silapentadecan-15-yl)carbamate (Boc-NH-PEG₂-C₅-OTBS, 20)—Yield: 46%. ¹H NMR (400 MHZ, CDCl₃): δ3.56 (m, 8H), 3.44 (t, 2H), 3.29 (q, 2H), 1.55 (m, 3H), 1.42 (s, 9H), 1.36 (m, 2H), 0.87 (s, 9H), 0.02 (s, 6H). ¹³C NMR (101 MHZ, CDCl₃): δ156.11, 71.57, 50.39, 70.32, 70. 12, 40.46, 37.75, 29.49, 28.58 (3C), 26.08 (3C), 22.45, 18.46, −5.17 (2C). HRMS (m/z): [M+H]⁺ calcd. for C₂₀H₄₃NO₅Si⁺, 406.2983; found, 406.2983.

tert-Butyl (2-(2-(4-methoxybutoxy)ethoxy)ethyl)carbamate (Boc-NH-PEG₂-C₃-OTBS, 21)—Yield: 43%. ¹H NMR (400 MHZ, CDCl₃): δ3.54 (m, 6H), 3.45 (m, 2H), 3.35 (m, 2H), 3.28 (s, 3H), 2.26 (m, 2H), 1.61 (tdd, 4H), 1.40 (s, 9H). HRMS (m/z): [M+H]⁺ calcd. for C₁₄H₃₀NO₅⁺, 292.2118; found, 292.2114.

6.1.5 Boc-NH-PEG₂-C₆ (22)

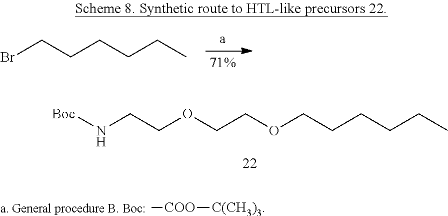

According to Tang et al. (2017). 6-bromon-hexane (1.02 mg, 7.32 mmol, 1.2 eq.) was used in an Williamson ether synthesis reaction carried out according to general procedure B. The reaction was completed after 16 h. Aq. work-up and flash column purification (25 g SiO₂, 10 to 50% EtOAc in n-hexane, 10 CV) delivered 22 as a colourless oil (1.06 g, 3.66 mmol).

tert-Butyl N-(2-(2-(6-hexyl)ethoxy)ethyl)carbamate (22)—Yield: 71%. ¹H NMR (400 MHZ, CDCl₃): δ3.60 (m, 6H), 3.49 (dd, 2H), 3.35 (m, 2H), 1.61 (dq, 2H), 1.47 (s, 9H), 0.94 (t, 3H). HRMS (m/z): [M+Na]⁺ calcd. for C₁₅H₃₁NO₄Na⁺, 312.2145; found, 312.2140.

### 6.1.6 Fluorophore Labeling

a. Deprotection

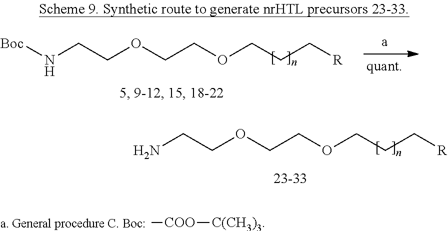

Primary amines were generated by Boc protecting group cleavage according to general procedure C. The generated compounds were well-dried but used without further purification for fluorophore coupling. All compounds were obtained in quantitative yields.

TABLE 5
Deprotection of potential nrHTL precursors used in this work

| Reaction | Abbreviation R | Full name |
|---|---|---|
| 9 → 23 | C₄-MSA | N-(4-(2-(2-aminoethoxy)ethoxy)butyl)meth- |
| | | anesulfonamide |
| 10 → 24 | C₅-MSA | N-(5-(2-(2-aminoethoxy)ethoxy)pentyl)meth- |
| | | anesulfonamide |
| 11 → 25 | C₄—F₃-MSA | N-(4-(2-(2-aminoethoxy)ethoxy)bu- |
| | | tyl)triflamide |
| 12 → 26 | C₄-NAC | N-(4-(2-(2-aminoethoxy)ethoxy)bu- |
| | | tyl)acetamide |
| 5 → 27 | C₄—N₃ | 2-(2-(4-azidobutoxy)ethoxy)ethan-1-amine |
| 15 → 28 | C₅-MA | 5-(2-(2-aminoethoxy)ethoxy)-N-meth- |
| | | ylpentan-1-amine |
| 18 → 29 | C₃—OH | 5 -(2-(2-aminoethoxy)ethoxy)propan-1-ol |
| 19 → 30 | C₄—OH | 5-(2-(2-aminoethoxy)ethoxy)butan-1-ol |
| 20 → 31 | C₅—OH | 5-(2-(2-aminoethoxy)ethoxy)pentan-1-ol |
| 21 → 32 | C₄-OMe | 2-(2-(4-methoxybutoxy)ethoxy)ethan-1- |
| | | amine |
| 22 →33 | C₆ | 2-(2-(hexyloxy)ethoxy)ethan-1-amine |

Reaction number, nomenclature and full name of nrHTL linkers (H₂N-PEG₂-R) synthesized in this work.

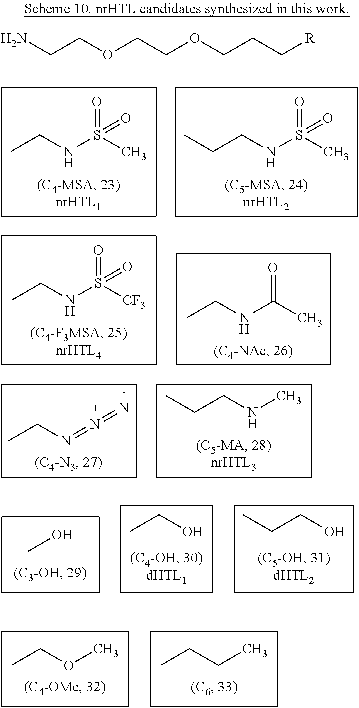

N-(4-(2-(2-aminoethoxy)ethoxy)butyl)methanesulfonamide (H₂N-PEG₂-C₄-MSA, 23)—¹H NMR (400 MHZ, MeOD): δ3.66 (m, 6H), 3.53 (t, 2H), 3.11 (m, 4H), 2.92 (s, 3H), 1.64 (m, 4H). ¹³C NMR (101 MHZ, MeOD): δ71.20, 70.72, 70.48, 43.17, 40.05, 39.01, 27.32, 27.02. HRMS (m/z): [M+H]⁺ calcd. for C₉H₂₂N₂O₄S, 255.1373; found, 255.1370.

N-(5-(2-(2-aminoethoxy)ethoxy)pentyl)methanesulfonamide (H₂N-PEG₂-C₅-MSA, 24)—¹H NMR (400 MHZ, MeOD): δ3.66 (m, 6H), 3.51 (t, 2H), 3.12 (m, 2H), 3.09 (t, 2H), 2.91 (s, 3H), 1.60 (m, 4H), 1.46 (m, 2H). ¹³C NMR (101 MHZ, MeOD): δ71.53, 70.68, 70.50, 67.22, 43.26, 40.04, 38.91, 30.26, 29.47, 23.56. HRMS (m/z): [M+H]⁺ calcd. for C₁₀H₂₄N₂O₄S, 269.1530 found, 269.1528.

N-(4-(2-(2-aminoethoxy)ethoxy)butyl)trifluoromethanesulfonamide (H₂N-PEG₂-C₄-F₃MSA, 25)—¹H NMR (400 MHZ, CDCl₃): δ3.68 (t, 2H), 3.60 (m, 2H), 3.55 (m, 2H), 3.52 (m, 2H), 3.27 (dq, 4H), 1.71 (td, 4H). ¹³C NMR (101 MHZ, CDCl₃): δ119.92 (q, 1C), 71.18, 70.20, 44.30, 30.05, 31.08, 27.11, 27.62. 19F NMR (377 MHZ, CDCl₃): δ-77.6 HRMS (m/z): [M+H]⁺ calcd. for C₉H₁₉F₃N₂O₄S, 309.1090 found, 309.1089.

N-(4-(2-(2-aminoethoxy)ethoxy)butyl)acetamide (H₂N-PEG₂-C₄-NAc, 26)—¹H NMR (400 MHZ, MeOD): δ3.65 (m, 6H), 3.51 (t, 2H), 3.18 (t, 2H), 3.12 (t, 2H), 1.93 (s, 3H), 1.59 (m, 4H). ¹³C NMR (101 MHZ, MeOD): δ173.32, 71.86, 71.32, 71.09, 67.81, 40.65, 40.20, 27.85, 27.01, 22.48. HRMS (m/z): [M+H]⁺ calcd. for C₁₀H₂₂N₂O₃, 219.1703 found, 219.1701.

2-(2-(4-azidobutoxy)ethoxy)ethan-1-amine (27)—¹H NMR (H₂N-PEG₂-C₄-N₃, 400 MHZ, MeOD): δ3.66 (m, 6H), 3.53 (ddt, 2H), 3.34 (m, 2H), 3.12 (m, 2H), 1.67 (tq, 4H). HRMS (m/z): [M+H]⁺ calcd. for C₈H₁₈N₄O₂, 203.1503 found, 203.1503.

5-(2-(2-aminoethoxy)ethoxy)-N-methylpentan-1-amine (H₂N-PEG₂-C₅-MA, 28)—¹H NMR (400 MHZ, MeOD): δ3.51 (q, 4H), 3.42 (m, 4H), 2.70 (t, 2H), 2.49 (t, 2H), 2.30 (s, 3H), 1.49 (m, 4H), 1.32 (m, 2H). ¹³C NMR (101 MHZ, MeOD): δ73.46, 72.16, 71.26, 71.12, 52.51, 42.09, 35.97, 30.51, 29.96, 24.85. HRMS (m/z): [M+H]⁺ calcd. for C₁₀H₂₄N₂O₂, 205.1911 found, 205.1910

5-(2-(2-aminoethoxy)ethoxy)propan-1-ol (H₂N-PEG₂-C₃-OH, 29)—¹H NMR (400 MHZ, MeOD): δ4.46 (t, 2H), 3.75 (t, 2H), 3.63 (m, 6H), 3.28 (h, 2H), 2.02 (p, 2H). ¹³C NMR (101 MHZ, MeOD): δ70.10, 67.11, 66.05, 65.19, 40.37, 28.11. HRMS (m/z): [M+H]⁺ calcd. for C₇H₁₇NO₃, 164.1281 found, 164.1281.

5-(2-(2-aminoethoxy)ethoxy)butan-1-ol (H₂N-PEG₂-C₄-OH, 30)—¹H NMR (400 MHZ, MeOD): δ3.66 (m, 2H), 3.57 (t, 2H), 3.53 (t, 2H), 3.12 (m, 2H), 1.63 (m, 4H). HRMS (m/z): [M+H]⁺ calcd. for C₈H₁₉NO₃, 178.1438 found, 178.1439.

5-(2-(2-aminoethoxy)ethoxy)pentan-1-ol (H₂N-PEG₂-C₄-OH, 31)—¹H NMR (400 MHZ, MeOD): δ4.33 (t, 2H), 3.71 (m, 2H), 3.59 (ddt, 4H), 3.45 (t, 2H), 3.14 (m, 2H), 1.75 (dt, 2H), 1.59 (h, 2H), 1.42 (m, 2H). ¹³C NMR (101 MHZ, MeOD): δ71.01, 70.31, 69.90, 68.16, 39.70, 28.89, 27.85, 22.18. HRMS (m/z): [M+H]⁺ calcd. for C₉H₂₁NO₃, 192.1594 found, 192.1593.

2-(2-(4-methoxybutoxy)ethoxy)ethan-1-amine (H₂N-PEG₂-C₄-OMe, 32)—¹H NMR (400 MHZ, MeOD): δ3.76 (m, 2H), 3.68 (m, 4H), 3.56 (m, 4H), 3.44 (t, 2H), 3.43 (s, 3H), 1.66 (ddp, 4H). HRMS (m/z): [M+H]⁺ calcd. for C₉H₂₁NO₃, 192.1594 found, 192.1594.

2-(2-(hexyloxy)ethoxy)ethan-1-amine (H₂N-PEG₂-C₆, 33)—¹H NMR (400 MHZ, MeOD): δ3.60 (m, 6H), 3.49 (t, 2H), 3.07 (t, 2H), 1.58 (dq, 42H), 1.35 (m, 6H), 0.94 (t, 3H). HRMS (m/z): [M+H]⁺ calcd. for C₁₀H₂₃NO₂, 190.1801found, 190.1802

b. Coupling to 6-carboxy rhodamine-based Fluorophores

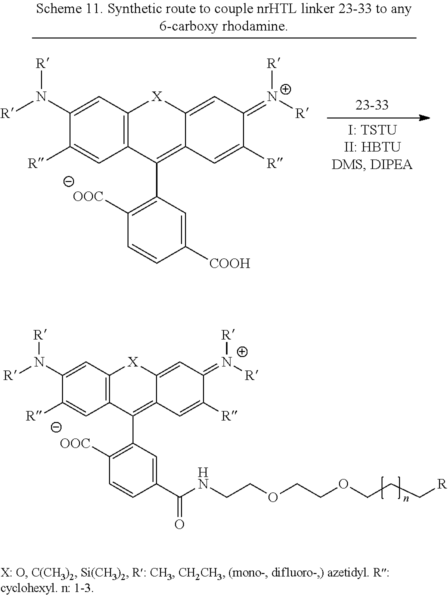

Coupling condition I: The following 6-carboxy modified rhodamine-based fluorescent dyes were coupled to the respective amino linker with the coupling reagent N,N,N′,N′-Tetramethyl-O-(N-succinimidyl)uronium tetrafluoroborate (TSTU) in dry DMSO: Tetramethyl rhodamine (TMR), carbopyronine (CPy), silicon rhodamine (SiR), 3-(N,N-dimethylaminosulfonamide) tetramethyl rhodamine (MaP555), F₄-bisazetidine rhodamine (F₄-BAR, JF₅₂₅), F₄-bisazetidine carbopyronine (F₄-BACPy, JF₅₈₅).

Coupling condition II: The following 6-carboxy modified rhodamine-based fluorescent dyes were coupled to the respective amino linker with the coupling reagent (2-(1H-benzotriazol-1-yl)-1,1,3,3-tetramethyluronium hexafluorophosphate (HBTU) in dry DMSO: (N,N-dimethylaminosulfonamide) carbopyronine (MaP618), bisazetidine silicon rhodamine (BASiR, JF₆₅₆), F₂-bisazetidine silicon rhodamine (F₂-BASiR, JF₆₃₅), F₄-bisazetidine silicon rhodamine (F₄-BASiR, JF₆₁₅), Atto565.

1 eq. 6-Carboxy fluorophore was dissolved in ˜100 μL/μmol dry DMSO-d₆. The mixture was added on top of 1.2 eq. coupling reagent (I: TSTU, II: HBTU) and 4-10 eq. diisopropylethylamine (DIPEA) were spiked into the solution. It was left stirring for at least 10 min at rt. The formation of the respective N-hydroxysuccinimid (NHS) or hydroxybenzotriazole (HOBT) intermediate (“active ester”) was monitored by LC-MS (NHS: M_{Dye}+98 m/z, HOBT: M_{Dye}+133 m/z). Meanwhile, the amine linker 23-33 was dissolved in the same amount of dry DMSO-de with 4-10 eq. DIPEA. Both solutions were mixed after full activation of the dye and left stirring for 1-4 h at 35 to 50° C.

After completion of the reaction excess of MeCN with 20% H₂O and 0.1% acetic acid was added and the mixture was purified by RP-HPLC.

TMR-23—¹H NMR (400 MHZ, CD₃CN): δ8.27 (d, 1H), 8.11 (d, 1H), 7.70 (d, 1H), 7.73 (d, 1H), 7.01 (d, 2H), 6.85 (dd, 2H), 6.78 (d, 2H), 3.53 (m, 8H), 3.38 (m, 2H), 3.19 (s, 12H), 2.97 (m, 2H), 2.82 (s, 3H), 1.51 (m, 4H).

TMR-24—¹H NMR (400 MHZ, CD₃CN): δ8.11 (d, 1H), 8.95 (dd, 1H), 7.58 (d, 1H), 7.32 (t, 1H), 6.78 (d, 2H), 6.62 (d, 4H), 3.52 (m, 4H), 3.45 (m, 4H), 3.32 (t, 2H), 3.05 (s, 12H), 2.95 (q, 2H), 2.82 (s, 3H), 1.44 (h, 4H), 1.28 (m, 2H).

TMR-25—¹H NMR (400 MHZ, CD₃CN): δ8.07 (m, 2H), 7.56 (s, 2H), 7.22 (s, 2H), 6.73 (d, 2H), 6.60 (m, 2H), 3.54 (m, 6H), 3.36 (t, 2H), 3.18 (t, 2H), 3.04 (s, 12H), 1.51 (m, 4H), 1.27 (m, 4H). ¹⁹F NMR (377 MHZ, CD₃CN): δ-79.62.

TMR-26—¹H NMR (400 MHZ, CD₃CN): δ9.14 (d, 1H), 8.96 (d, 1H), 8.58 (s, 1H), 8.38 (t, 1H), 7.89 (dd, 2H), 7.72 (d, 2H), 7.64 (s, 2H), 4.38 (m, 8H), 3.21 (td, 2H), 3.05 (s, 12H), 3.87 (q, 2H), 2.61 (s, 3H), 2.27 (dq, 2H).

TMR-27—¹H NMR (400 MHZ, CD₃CN): δ8.25 (d, 1H), 8.09 (dd, 1H), 7.67 (s, 1H), 7.31 (s, 2H), 6.98 (d, 2H), 6.80 (m, 4H), 3.52 (m, 8H), 3.38 (t, 2H), 3.27 (s, 12H), 3.16 (m, 2H), 1.54 (m, 4H).

TMR-28—¹H NMR (400 MHZ, CD₃CN): δ8.28 (dd, 1H), 8.10 (dd, 1H), 7.72 (t, 1H), 7.57 (d, 1H), 7.04 (t, 2H), 6.87 (ddd, 2H), 6.79 (m, 2H), 3.54 (m, 8H), 3.35 (t, 2H), 3.20 (d, 12H), 2.88 (t, 2H), 2.56 (s, 3H), 1.61 (p, 2H), 1.45 (m, 2H) 1.29 (m, 2H).

TMR-30—¹H NMR (400 MHZ, CD₃CN): δ8.32 (d, 1H), 8.14 (dd, 1H), 7.82 (t, 1H), 7.75 (d, 1H), 7.09 (d, 2H), 6.92 (dd, 2H), 6.83 (d, 2H), 3.54 (m, 8H), 3.39 (t, 2H), 3.36 (t, 2H), 3.23 (s, 12H), 1.52 (m, 2H), 1.44 (m, 2H).

TMR-31—¹H NMR (400 MHZ, CD₃CN): δ8.24 (d, 1H), 8.09 (dd, 1H), 7.67 (d, 1H), 7.36 (d, 1H), 6.97 (d, 2H), 6.81 (dd, 2H), 6.75 (d, 2H), 3.51 (m, 7H), 3.42 (t, 2H), 3.35 (t, 2H), 3.17 (s, 12H), 1.43 (tt, 4H), 1.28 (m, 4H).

TMR-33—¹H NMR (400 MHZ, CD₃CN): δ8.32 (d, 1H), 8.14 (dd, 1H), 7.74 (d, 1H), 7.58 (t, 1H), 7.07 (d, 2H), 6.91 (dd, 2H), 6.83 (d, 2H), 3.55 (m, 8H), 3.37 (t, 2H), 3.24 (s, 12H), 1.45 (q, 2H), 1.27 (m, 2H), 0.88 (t, 3H).

TABLE 6
Derivatization of various rhodamine-based dyes with HTL-like linkers.

| linker | Dye | LC [min] | Purity [%] | Chem. formula | [M]_{calcd.} | [M]_{found} |
|---|---|---|---|---|---|---|
| 23 | TMR | 6.94 | 95 | C₃₄H₄₃N₄O₈S | 667.2786 | 667.2786 |
| | SiR | 7.57 | 97 | C₃₆H₄₉N₄O₇SSi | 709.3086 | 709.3083 |
| | CPy | 7.12 | 95 | C₄₀H₅₁N₄O₈S | 747.3422 | 747.3418 |
| | Atto565 | 9.79 | 100 | C₃₇H₄₉N₄O₇S | 693.3316 | 693.3318 |
| | MaP555 | 7.83 | 95 | C₃₆H₄₈N₆O₉S₂ | 773.2997 | 773.3001 |
| | JF₅₂₅ | 6.23 | 99 | C₃₆H₃₉F₄N₄O₈S | 763.2419 | 763.2419 |
| | JF₅₈₅ | 10.77 | 95 | C₃₉H₄₅F₄N₄O₇S | 789.2940 | 789.2936 |
| | MaP618 | 12.08 | 88 | C₃₉H₅₄N₆O₈S₂ | 799.3517 | 799.3516 |
| | JF₆₁₅ | 6.50 | 95 | C₃₈H₄₅F₄N₄O₇SSi | 805.2709 | 805.2703 |
| | JF₆₃₅ | 10.48 | 98 | C₃₈H₄₇F₂N₄O₇SSi | 769.2897 | 769.2894 |
| | JF₆₅₆ | 9.84 | 97 | C₃₈H₄₉N₄O₇SSi | 733.3086 | 733.3091 |
| 24 | TMR | 6.53 | 99 | C₃₅H₄₅N₄O₈S | 681.2953 | 681.2952 |
| | SiR | 7.93 | 95 | C₃₇H₅₁N₄O₇SSi | 723.3242 | 723.3240 |
| | CPy | 7.87 | 96 | C₃₈H₅₁N₄O₇S | 707.3473 | 707.3473 |
| | Atto565 | N.D. | N.D. | C₃₈H₅₁N₄O₇S | 719.1990 | 719.1989 |
| 25 | TMR | 9.54 | 97 | C₃₄H₄₀N₄O₈F₃S | 721.2513 | 721.2513 |
| | SiR | 10.66 | 95 | C₃₆H₄₆N₄O₇F₃SSi | 763.2803 | 763.2805 |
| | MaP555 | 10.31 | 98 | C₃₆H₄₅F₃N₆O₉S₂ | 827.2714 | 827.2714 |
| | JF₅₈₅ | 12.76 | 96 | C₃₉H₄₂F₇N₄O₇S | 843.2657 | 843.2657 |
| 26 | TMR | 6.58 | 99 | C₃₅H₄₃N₄O₇ | 631.3126 | 631.3121 |
| | SiR | 7.64 | 98 | C₃₇H₄₉N₄O₆Si | 673.3416 | 673.3416 |
| 27 | TMR | 8.60 | 96 | C₃₃H₃₉N₆O₆ | 615.2926 | 615.2926 |
| 28 | TMR | 6.71 | 100 | C₃₅H₄₅N₄O₆H⁺ | 309.1703 | 309.1703 |
| | SiR | 8.73 | 100 | C₃₇H₅₁N₄O₅SiH⁺ | 330.1848 | 330.1845 |
| 29 | TMR | 6.49 | 99 | C₃₂H₃₈N₃O₇ | 576.2704 | 576.2704 |
| | SiR | 7.36 | 98 | C₃₄H₄₄N₃O₆Si | 618.2994 | 618.2994 |
| 30 | TMR | 6.56 | 95 | C₃₃H₄₀N₃O₇Na⁺ | 444.0431 | 444.0428 |
| | SiR | 7.70 | 99 | C₃₅H₄₆N₃O₆Si | 632.3150 | 632.3153 |
| 31 | TMR | 6.96 | 96 | C₃₄H₄₂N₃O₇ | 604.3017 | 604.3016 |
| | SiR | 8.08 | 98 | C₃₆H₄₈N₃O₆Si | 646.3307 | 646.3306 |
| 32 | TMR | 8.02 | 99 | C₃₄H₄₂N₃O₇ | 604.3017 | 604.3016 |
| | SiR | 6.21 | 99 | C₃₆H₄₈N₃O₆Si | 646.3307 | 646.3307 |
| 33 | TMR | 10.60 | 95 | C₃₅H₄₄N₃O₆ | 602.3225 | 602.3219 |

1. A non-covalently-HaloTag-binding compound characterized by the general formula (I)
D-L-T (I), wherein
a. D is or comprises a functional moiety Z selected from
an organic dye moiety characterized by a molecular mass of between 300 g/mol and 1300 g/mol, particularly a fluorescent organic dye moiety;
an affinity purification ligand,
a pharmaceutical drug or a pharmaceutical drug candidate,
an oligopeptide or a polypeptide,
a nanoparticle,
a solid surface or a matrix polymer,
a nucleic acid oligomer,
a carbohydrate,
a lipid,
a spectroscopic probe,
a sensor, particularly a fluorescent sensor,
a natural product, and
a synthetic ligand binding to a biomolecule with an affinity of at least 100 μM,
or
b. D is a linkable moiety selected from
an unprotected or protected amine moiety,
a sulfonamide moiety,
an N₃ moiety,
an alkyne moiety,
a carboxylic acid or an activated form of a carboxylic acid, particularly an N-hydroxysuccinimide moiety,
an ester moiety,
an aldehyde,
a thiol, and
an isothiocyanate;
L is a linear linker of 10-15 atoms in length, particularly wherein L is 10-11 atoms in length,
wherein L comprises, particularly L essentially consists of, alkyl, trans-alkylene, and/or ether moieties and optionally one or several methyl substituents,
particularly L is an unbranched linker that does not comprise methyl substituents;
T is a moiety selected from the group comprising methylamine, monofluormethylamine, difluormethylamine, trifluormethylamine, methylsulfonamide, monofluormethylsulfonamide, difluormethylsulfonamide, trifluormethylsulfonamide, azide, acetamide, monofluoracetamide, difluoracetamide, and trifluoracetamide;
particularly T is a moiety selected from the group comprising methylamine, trifluormethylsulfonamide and methylsulfonamide.

2. A non-covalently-HaloTag-binding compound characterized by the general formula (I)
D-L-T (I), wherein
a. D is or comprises a functional moiety Z selected from
an organic dye moiety characterized by a molecular mass of between 300 g/mol and 1300 g/mol, particularly a fluorescent organic dye moiety;
an affinity purification ligand,
a pharmaceutical drug or a pharmaceutical drug candidate,
an oligopeptide or a polypeptide,
a nanoparticle,
a solid surface or a matrix polymer,
a nucleic acid oligomer,
a carbohydrate, and
a lipid,
a spectroscopic probe,
a fluorescent sensor,
a natural product,
a synthetic ligand binding to a biomolecule with an affinity of at least 100 μM,
L is a linear linker of 10-15 atoms in length, particularly wherein L is 10-11 atoms in length,
wherein L comprises, particularly L essentially consists of, alkyl, trans-alkylene, and/or ether moieties and optionally one or several methyl substituents,
particularly L is an unbranched linker that does not comprise methyl substituents;
T is a moiety selected from the group comprising methylamine, monofluormethylamine, difluormethylamine, trifluormethylamine, methylsulfonamide, monofluormethylsulfonamide, difluormethylsulfonamide, trifluormethylsulfonamide, azide, acetamide, monofluoracetamide, difluoracetamide, and trifluoracetamide, and hydroxyl,
particularly T is a moiety selected from the group comprising methylamine, methylsulfonamide, trifluormethylsulfonamide and hydroxyl.

3. The non-covalently-HaloTag-binding compound according to claim 2, wherein T is hydroxyl.

4. The compound according to claim 1, wherein D comprises a linking moiety X which connects the functional moiety Z to L, particularly wherein X is selected from an amide, a secondary amine, a 1,2,3-triazole, an ester, a sulfonamide, an ether, a thioether, a thiourea, an urea, and a carbamate.

5. The compound according to claim 1, wherein L is a linear unbranched alkyl chain, comprising 1, 2, or 3 moieties independently selected from ether and trans-alkylene.

6. The compound according to claim 1, wherein the compound is characterized by the one of the general formulas (II), (III), (IV), (V) or (VI), particularly of the general formula (II), 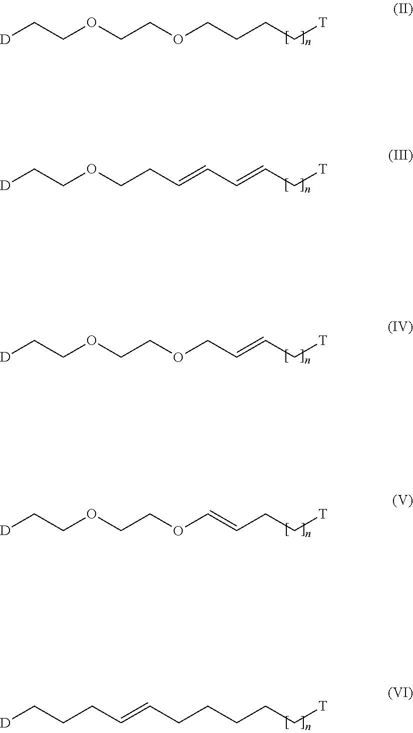
wherein n is an integer selected from 1, 2, 3, and 4, particularly n is 1 or 2, more particularly n is 1.

7. The compound according to claim 1, wherein Z is a fluorophore, particularly a fluorophore selected from a rhodamine, a silicon rhodamine, a fluorescein, a Janelia Fluor dye, an olefinic silicon rhodamine derivative with an exocyclic double bond, a MaP dye, a carbopyronine, a carbocyanine (particularly a Cy3, or a Cy5 dye), a pyrene, a Bodipy fluorophore, a coumarine, a rhodol, and an Alexa dye.

8. The compound according to claim 1, wherein Z is of the general formula (VII) or of the general formula (VIII) 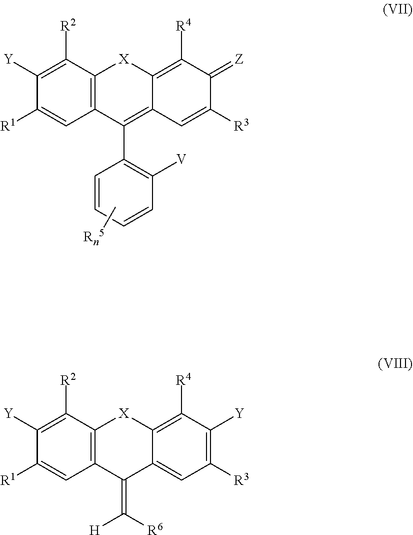
wherein
V is selected from —C(═O)W, —CH₃, and —CH₂OH, wherein W is OH or NR^{W1}R^{W2} with R^{W1} and R^{W2} being independently selected from H, unsubstituted or amino- or hydroxy-substituted C₁-C₈ alkyl, CN, SO₂NR^{S1}R^{S2} and SO₂R^{S} with R^{S} being unsubstituted or amino- or hydroxy-substituted C₁-C₆ alkyl, and with R^{S1} and R^{S2} being independently selected from H and unsubstituted or amino- or hydroxy-substituted C₁-C₆ alkyl;
R¹, R², R³, and R⁴ are independently selected from H and an unsubstituted or hydroxy-, amino-, halogen-, and/or carboxy-substituted hydrocarbon moiety selected from C₁-C₈ alkyl, C₃-C₈ cycloalkyl, C₁-C₄ acyl, C₇-C₁₂ alkylaryl, an unsubstituted phenyl or a phenyl substituted by any one or several of the following substituents:
unsubstituted C₁-C₄ alkyl, halogen, C₁-C₄ oxyalkyl, COOH, COOR^{C}, CONR^{C}₂, with R^{C} being selected from H and unsubstituted or amino- or hydroxy-substituted C₁-C₈ alkyl;
or R¹, R², R³, and/or R⁴ form a ring structure as described below;
R⁵ is selected from an amine, carbonyl, ester, amide, sulfonamide, and a hydrocarbon moiety selected from C₁-C₈ alkyl, C₃-C₈ cycloalkyl, C₇-C₁₂ alkylaryl, and phenyl,
wherein R⁵ is unsubstituted or hydroxy-, amino-, halogen-, and/or carboxy-substituted;
n is an integer selected from 0, 1, and 2;
R⁶ is selected from an amine, carbonyl, ester, amide, sulfonamide, and a hydrocarbon moiety selected from C₁-C₈ alkyl, C₃-C₈ cycloalkyl, C₇-C₁₂ alkylaryl, and phenyl,
wherein R⁶ is unsubstituted or hydroxy-, amino-, halogen-, and/or carboxy-substituted;
X is selected from O, S, Se, TeO, POR^{X}, POOR^{X}, SO₂, NR^{X}, CR^{X}₂, SiR^{X}₂, GeR^{X}₂, and SnR^{X}₂, with each R^{X} being independently selected from H and an unsubstituted or substituted (particularly unsubstituted or hydroxy-, amino-, halogen-, and/or carboxy-substituted) moiety selected from C₁-C₁₂ alkyl, C₃-C₈ cycloalkyl, C₂-C₁₂ alkenyl, C₂-C₁₂ alkynyl, C₇-C₁₂ alkylaryl, phenyl and 5- or 6-membered heteroaryl, or two RX moieties form a four-, five-, six- or seven-membered unsubstituted or amino-, hydroxy- and/or halogen substituted alkyl ring;
particularly X is selected from O, CR^{X}₂, and SiR^{X}₂;
Y is OH or NR^{Y1}R^{Y2},
Z is O or N⁺R^{Y1}R^{Y2}
with
R^{Y1}, and R^{Y2} each independently selected from H, an unsubstituted or hydroxy-, amino-, halogen-, and/or carboxy-substituted hydrocarbon moiety selected from C₁-C₈ alkyl, C₃-C₈ cycloalkyl, C₁-C₄ acyl, C₇-C₁₂ alkylaryl, an unsubstituted phenyl or a phenyl substituted by any one or several of the following substituents: unsubstituted C₁-C₄ alkyl, halogen, C₁-C₄ oxyalkyl, COOH, COOR^{YC}, CONR^{YC}₂, with R^{YC} being selected from H and unsubstituted or amino- or hydroxy-substituted C₁-C₈ alkyl; or
R^{Y1} and R^{Y2} together are a C₃-C₆ unsubstituted or hydroxy-, amino-, halogen-, alkoxy- and/or carboxy-substituted alkyl forming a 4-7-membered ring structure with Y; or
one of R^{Y1} and R^{Y2}, or both R^{Y1} and R^{Y2}, together with R¹ and/or R², and/or R³ and/or R⁴, respectively, form an unsubstituted or hydroxy-, amino-, halogen-, carboxy- and/or aryl-substituted 4-7-membered alkyl or alkylene ring;
wherein Z is connected to L via a substituent selected from R¹, R², R³, R⁴, R⁵, R⁶, Y, Z and V.

9. A kit comprising
a. a catalytically functional HaloTag polypeptide, wherein optionally said HaloTag polypeptide is attached to a polypeptide of interest;
or
an expression vector encoding said catalytically functional HaloTag polypeptide;
and the kit further comprising a compound according to claim 1;
or
b. a D106 mutant HaloTag polypeptide, wherein position D106 of the HaloTag7 sequence (position 103 designated as X in SEQ ID No 001) is exchanged for a proteinogenic amino acid different from D,
particularly wherein the mutant HaloTag polypeptide comprises a mutation selected from D106A, D106G, and D106T, more particularly wherein the mutant HaloTag polypeptide comprises a D106A mutation,
or a D106 mutant HaloTag polypeptide, wherein, in a HaloTag polypeptide sequence homologous to HaloTag7, at a position homologous to D106 of HaloTag7, D is exchanged for a proteinogenic amino acid different from D,
particularly wherein said position is changed to A, G or T, more particularly wherein said position is changed to A,
wherein optionally said D106 mutant HaloTag polypeptide is attached to a polypeptide of interest;
or
an expression vector encoding said D106 mutant HaloTag polypeptide;
and the kit further comprising a compound characterized by the general formula (I)
D-L-T (I), wherein
D is or comprises a functional moiety Z selected from
an organic dye moiety characterized by a molecular mass of between 300 g/mol and 1300 g/mol, particularly a fluorescent organic dye moiety;
an affinity purification ligand,
a pharmaceutical drug or a pharmaceutical drug candidate,
an oligopeptide or a polypeptide,
a nanoparticle,
a solid surface or a matrix polymer,
a nucleic acid oligomer,
a carbohydrate, and
a lipid,
a spectroscopic probe,
a fluorescent sensor,
a natural product,
a synthetic ligand binding to a biomolecule with an affinity of at least 100 μM,
L is a linear linker of 10-15 atoms in length, particularly wherein L is 10-11 atoms in length,
wherein L comprises, particularly L essentially consists of, alkyl, trans-alkylene, and/or ether moieties and optionally one or several methyl substituents,
particularly L is an unbranched linker that does not comprise methyl substituents;
T is a moiety selected from the group comprising methylamine, monofluormethylamine, difluormethylamine, trifluormethylamine, methylsulfonamide, monofluormethylsulfonamide, difluormethylsulfonamide, trifluormethylsulfonamide, azide, acetamide, monofluoracetamide, difluoracetamide, and trifluoracetamide, and hydroxyl,
particularly T is a hydroxyl.

10. A method for binding a HaloTag polypeptide in a sample, said method comprising the steps of:
a. providing an aqueous sample comprising a HaloTag polypeptide;
b. contacting the sample with a compound comprising a fluorescent organic dye moiety according to claim 1;
c. illuminating the sample with light of an excitation wavelength appropriate for exciting said fluorescent organic dye moiety, and recording light emitted from said sample, particularly at an appropriate emission wavelength 2, more particularly a λ close to a maximum of the emission spectrum of 400-800 nm,
d. optionally repeating step c.

11. A method for labelling a HaloTag polypeptide in a sample, said method comprising the steps of:
a. providing an aqueous sample comprising a HaloTag polypeptide attached to a luciferase;
b. contacting the sample with a compound comprising a fluorescent organic dye moiety according to claim 1;
c. contacting the sample with a luciferase substrate and recording light emitted from said sample, particularly at an appropriate emission wavelength λ, more particularly a λ close to a maximum of the emission spectrum of 400-800 nm;
d. optionally repeating step c.

12. The method according to claim 10, wherein a first and a second HaloTag polypeptide are specifically labelled,
wherein the first HaloTag polypeptide is a wildtype polypeptide and wherein the second HaloTag polypeptide is a HaloTag polypeptide mutant, wherein D106 of HaloTag7 or the analogous D of a different HaloTag, is exchanged for a different proteinogenic amino acid,
particularly wherein the second HaloTag polypeptide comprises a mutation selected from D106A, D106G, and D106T, or the analogous mutation of D, more particularly wherein the second HaloTag polypeptide comprises a D106A mutation, or the analogous mutation of D, and
wherein a first and a second compound comprising a first fluorescent organic dye moiety and a second fluorescent organic dye, respectively, are employed,
wherein for the first compound T is a moiety selected the group comprising of methylamine, methylsulfonamide, monofluormethylsulfonamide, difluormethylsulfonamide, trifluormethylsulfonamide, azide, and acetamide, monofluoracetamide, difluoracetamide, and trifluoracetamide,
particularly wherein T is selected from methylamine, methylsulfonamide, and trifluormethylsulfonamide,
and
wherein for the second compound T is OH, and
wherein the emission wavelength of the first fluorescent organic dye moiety is different from the emission wavelength of the second fluorescent organic dye moiety.

13. A D106 mutant HaloTag polypeptide, wherein, in a HaloTag7 polypeptide sequence or a HaloTag polypeptide sequence homologous to HaloTag7, at position D106 of HaloTag7 or at a position homologous to D106 of HaloTag7, D is exchanged for a proteinogenic amino acid different from D,
particularly wherein said position is changed to G, A, V, I, L, C, S, T, N, or E,
more particularly wherein said position is changed to A, G or T, even more particularly wherein said position is changed to A,

14. The D106 mutant HaloTag polypeptide according to claim 13 comprising or essentially consisting of a sequence selected from SEQ ID NO 001 to SEQ ID NO 007,
wherein X is selected from any proteinogenic amino acid except D,
particularly wherein X is selected from G, A, V, I, L, C, S, T, N, and E, more particularly wherein X is selected from A, G, and T, most particularly wherein X is A.

15. A nucleic acid sequence encoding the D106 mutant polypeptide according to claim 13.


SEQUENCE LISTING (13 sequences)

SEQ ID NO: 1 - protein, 292 aa, Artificial Sequence
  HaloTag
  MISC_FEATURE (103)..(103): any proteinogenic amino acid
IGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHXWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEI

SEQ ID NO: 2 - protein, 296 aa, Artificial Sequence
  HaloTag
  MISC_FEATURE (105)..(105): any proteinogenic amino acid
AEIGTGFPFDPHYVEVLGERMHYVDVGPRDGTPVLFLHGNPTSSYVWRNIIPHVAPTHRSIAPDLIGMGKSDKPDLGYFFDDHVRFMDAFIEALGLEEVVLVIHXWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNSKAVDIGPGLNLLQEDNPDLIGSEIARWLSTLEISG

SEQ ID NO: 3 - protein, 292 aa, Artificial Sequence
  HaloTag
  MISC_FEATURE (103)..(103): any proteinogenic amino acid
IGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHXWGSALGFHWAKRNPERVKGIAFMEFIRPIPTWDEWPEFARETFQAFRTTDVGRKLIIDHNVFIEGTLRMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEI

SEQ ID NO: 4 - protein, 294 aa, Artificial Sequence
  HOB
  MISC_FEATURE (103)..(103): any proteinogenic amino acid
IGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHXWGSALGFHWAKRNPERVKGIAFMEFIRPIPTWDEWPKFARKTFQAFRTKKVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 5 - protein, 308 aa, Artificial Sequence
  cpHalo
  MISC_FEATURE (273)..(273): any proteinogenic amino acid
GGGFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEIGGTGGSGGTGGSGGSIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHXWGSALGFHWAKRNPERVKGIAFMEFIRPIPTWDEW

SEQ ID NO: 6 - protein, 308 aa, Artificial Sequence
  cpHalo
  MISC_FEATURE (261)..(281): any proteinogenic amino acid
GGGDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEIGGTGGSGGTGGSGGSIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHXWGSALGFHWAKRNPERVKGIAFMEFIRPIPTWDEWPEFARETFQAFR

SEQ ID NO: 7 - protein, 309 aa, Artificial Sequence
  cpHalo
  MISC_FEATURE (261)..(261): any proteinogenic amino acid
GGGDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEIGGTGGSGGTGGSGGSIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHXWGSALGFHWAKRNPERVKGIAFMEFIRPIPTWDEWPEFARETFQAFRT

SEQ ID NO: 8 - DNA, 876 bp, Artificial Sequence
  HaloTag DNA
  misc_feature (308)..(308): m equals a or c
atcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgmctggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatt

SEQ ID NO: 9 - DNA, 927 bp, Artificial Sequence
  HaloTag DNA
  misc_feature (359)..(359): m equals a or c
catcaccaccatcatcaccatcaccaccacgaaaacctgtattttcagggcatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgmctggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcataacgtttttatcgagggtacgctgctgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatt

SEQ ID NO: 10 - DNA, 939 bp, Artificial Sequence
  HOB DNA
  misc_feature (362)..(362): m equals a or c
atgcatcaccaccatcatcaccatcaccaccacgaaaacctgtattttcagggcattggcaccggttttccgtttgatccgcattatgttgaagttctgggtgaacgtatgcattatgtggatgttggtccgcgtgatggtacaccggttctgtttctgcatggtaatccgaccagcagctatgtttggcgtaacattattccgcatgttgcaccgacccatcgttgtattgcaccggatctgattggtatgggtaaaagcgataaacctgatctgggctattttttcgatgatcatgtgcgttttatggacgcctttattgaagcactgggtctggaagaagttgtgctggttattcatgmttggggtagcgcactgggttttcattgggcaaaacgtaatccggaacgtgttaaaggtattgcctttatggaatttattcgtccgattccgacctgggatgaatggccgaaatttgcacgtaaaacctttcaggcatttcgcaccaaaaaagttggtcgcaaactgattattgaccagaacgtttttatcgaaggcaccctgccgatgggtgttgttcgtccgctgaccgaagttgaaatggatcattatcgtgaaccgtttctgaatccggttgatcgcgaaccgctgtggcgttttccgaatgaactgccgattgccggtgaacctgcaaatattgttgcactggttgaagagtatatggattggctgcatcagagtccggttccgaaactgctgttttggggcacaccgggtgttctgattccgcctgcagaagcagcacgtctggcaaaaagcctgccgaattgtaaagcagttgatattggtccgggtctgaatctgctgcaagaagataatccagatctgatcggtagtgaaattgcacgttggctgagcaccctggaaattagcggctaa

SEQ ID NO: 11 - DNA, 972 bp, Artificial Sequence
  HOB DNA
  misc_feature (866)..(866): m equals a or c
catcaccaccatcatcaccatcaccaccacgaaaacctgtattttcagggcggtggctttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagattggtggcaccggtggctctggcggtaccggtggctctggcggttctatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgmctggggctccgctctgggtttccactgggccaagcgcaatccagagcgcgtcaaaggtattgcatttatggagttcatccgccctatcccgacctgggacgaatgg

SEQ ID NO: 12 - DNA, 972 bp, Artificial Sequence
  cpHalo DNA
  misc_feature (830)..(830): m equals a or c
catcaccaccatcatcaccatcaccaccacgaaaacctgtattttcagggcggtggcgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagattggtggcaccggtggctctggcggtaccggtggctctggcggttctatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgmctggggctccgctctgggtttccactgggccaagcgcaatccagagcgcgtcaaaggtattgcatttatggagttcatccgccctatcccgacctgggacgaatggccagaatttgcccgcgagaccttccaggccttccgc

SEQ ID NO: 13 - DNA, 975 bp, Artificial Sequence
  cpHalo DNA
  misc_feature (830)..(830): m equals a or c
catcaccaccatcatcaccatcaccaccacgaaaacctgtattttcagggcggtggcgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagattggtggcaccggtggctctggcggtaccggtggctctggcggttctatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgmctggggctccgctctgggtttccactgggccaagcgcaatccagagcgcgtcaaaggtattgcatttatggagttcatccgccctatcccgacctgggacgaatggccagaatttgcccgcgagaccttccaggccttccgcacc